(19) United States
(12) Patent Application Publication
(10) Pub. No.: US 2026/0041669 A1
(43) Pub. Date: Feb. 12, 2026

(54) SOLID FORMS OF A CDK INHIBITOR

(71) Applicant: Relay Therapeutics, Inc., Cambridge, MA (US)

(72) Inventors: Andre Lescarbeau, Somerville, MA (US); Vishnu Vardhan Reddy Karnati, Littleton, MA (US); Ka Ning Yip, Franklin, MA (US); Seema Bag, Acton, MA (US); Osama Suleiman, Cambridge, Cambridgeshire (GB); Sanesh Mistry, Cambridge, Cambridgeshire (GB); Christopher Daniel Scott, Cambridge, Cambridgeshire (GB)

(21) Appl. No.: 19/101,235

(22) PCT Filed: Aug. 10, 2023
(86) PCT No.: PCT/US2023/029918

§ 371 Date: Feb. 4, 2025

Related U.S. Application Data
(60) Provisional application No. 63/397,120, filed on Aug. 11, 2022.

(51) Int. Cl. A61K 31/4155; C07D 405/14

(52) U.S. Cl. CPC A61K 31/4155; C07D 405/14

(57) ABSTRACT

The disclosure is in part directed to crystalline forms of (3R,5R)-5-(5-(3-(2,2-difluoroethoxy)-1-methyl-1H-pyrazole-5-carboxamido)-1H-pyrazol-3-yl)tetrahydrofuran-3-yl bicyclo[1.1.1]pentan-1-ylcarbamate, its solvates, its cocrystals, and variants thereof.

### BACKGROUND

Cyclin-dependent kinases (CDKs) are a family of serine/threonine kinases that are regulated by direct binding to cyclins. The initially-discovered CDKs (CDK1, CDK2, CDK4, CDK6) bind to cognate cyclins during specific cell cycle phases, activating their kinase activity and promoting cell cycle progression (Malumbres M. Genome Biology 2014). Related CDK family members (CDK7, CDK8, CDK9, CDK12, CDK13) are involved in other biological functions such as transcriptional control (Chou J., et al. Cancer Discovery 2020).

The cell cycle is initiated following mitogenic stimuli that signal for cyclin D expression, binding to CDK4/6, and kinase activation. The active CDK4/6-cyclin D complex mono-phosphorylates the retinoblastoma protein (RB), a tumor suppressor, to initiate cyclin E expression and formation of an active CDK2-cyclin E complex. Activated CDK2-cyclin E hyper-phosphorylates RB, triggering DNA replication, which is further promoted by CDK2-cyclin A. Finally, CDK1-cyclin B and CDK1-cyclin A coordinate segregation of duplicated DNA within the mother cell to complete cell division, and form two new daughter cells (Otto, T., and Sicinski, P. Nat Rev Cancer 2017).

As sustained cellular proliferation is one hallmark of cancer, alterations in pathways controlling cell cycle progression are frequently associated with cancer. Indeed, CCNE1 (gene encoding cyclin E1 protein) is among the most frequently amplified genes in variety of cancers including ovarian, endometrial, gastric, cervical, bladder, esophageal, lung, and breast cancers (Sanchez-Vega F., et al. Cell 2018; Cerami E., et al. Cancer Discovery 2012). The amplified CCNE1 gene, which leads to overexpression of cyclin E1 protein, is believed to be the oncogenic driver in those tumors due to increased CDK2-cyclin E activity. Notably, CCNE1 amplified or overexpressed tumor cells are dependent on CDK2 activity and thus provide the rationale for targeting CDK2 in this genetically defined patient population (McDonald E. R., et al Cell 2017; Au-Yeung G., et al. Clin Cancer Research 2016). Furthermore, CDK2 activation via Cyclin E1 amplification and overexpression is a common mechanism of resistance to several approved targeted therapies (such as CDK4/6 and HER2 modulators), and therefore supports combined targeting of CDK2 with other validated drivers in cancer (Turner N. C., et al. J Clin Oncology 2019; Herrera-Abreu M. T., et al. Cancer Research 2016; Scaltriti M., et al. PNAS 2011).

Multiple pan-CDK inhibitors with activity against CDK2 and other CDKs have shown evidence of clinical activity, however they have also shown significant hematopoietic and gastrointestinal toxicities likely due to their inhibition of CDK1 (Otto, T., and Sicinski, P., Nat. Review Cancer 2017; Kumar, K. S., et al. Blood 2015; Shapiro G. I., et al. Clin Cancer Research 2001). Whereas CDK2 activity may be dispensable for normal cell function, CDK1 activity is essential in all cells, especially in the highly proliferating cells of the gut and the hematopoietic system (Berthet C., et al. Current Biology 2003; Jayapal S. R., et al. Haematologica 2015; Santamaria D., et al. Nature 2007; Lu S., et al. Tox Sciences 2020).

Polymorphism is the ability of a substance to crystallize in more than one crystal lattice arrangement. Crystallization, or polymorphism, can influence many aspects of solid state properties of a drug substance. A crystalline form may differ considerably from an amorphous form, and different crystal forms of a substance may differ considerably from one another in many respects including solubility, dissolution rate and/or bioavailability. Generally, it is difficult to predict whether or not a given compound will form various crystalline solid state forms. It is even more difficult to predict the physical properties of these crystalline solid state forms. Further, it can be advantageous to have a crystalline form of a therapeutic agent for certain formulations, e.g., formulations suitable for subcutaneous use.

### SUMMARY

This disclosure is generally directed to the compounds of formulas I and solvates thereof, and crystalline forms thereof.

In one aspect, provided herein is a compound of Formula (I)

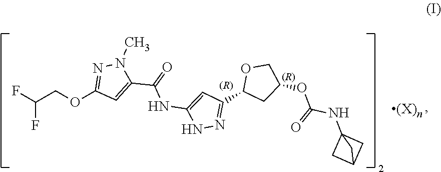

or a solvate thereof, wherein each of X, m, and n is independently as defined and described in embodiments herein. In some embodiments, a compound of Formula (I), or a solvate thereof, is a crystalline form as described herein.

In another aspect, provided herein is a pharmaceutical composition comprising a compound or a solvate thereof, or a crystalline form, as described herein, and a pharmaceutically acceptable excipient.

In another aspect, provided herein is a method of using a compound or a solvate thereof, or a crystalline form, or a pharmaceutical composition thereof, as described herein, for inhibiting CDK2 activity and for treating a disorder, disease, and/or condition as described herein.

### BRIEF DESCRIPTION OF THE DRAWINGS

FIG. 1 depicts an X-ray diffraction pattern of amorphous Compound I-1.

FIG. 2 depicts a ¹H NMR spectrum of amorphous Compound I-1 in DMSO-d6 at 400 MHz.

FIG. 3 depicts the characterization of amorphous Compound I-1 by thermogravimetric analysis (TGA).

FIG. 4 depicts the characterization of amorphous Compound I-1 by differential scanning calorimetry (DSC).

FIG. 5 depicts an X-ray diffraction pattern of Form A of Compound I-1.

FIG. 6 depicts an X-ray diffraction pattern of Form B of Compound I-1.

FIG. 7 depicts an X-ray diffraction pattern of Form A of Compound I-2.

FIG. 8 depicts a ¹H NMR spectrum of Form A of Compound I-2 in DMSO-d6 at 400 MHz.

FIG. 9 depicts the characterization of Form A of Compound I-2 by thermogravimetric analysis (above) and differential scanning calorimetry (below).

FIG. 10 depicts an X-ray diffraction pattern of Form A of Compound I-3

FIG. 11 depicts a ¹H NMR spectrum of Form A of Compound I-3 in DMSO-d6 at 400 MHz.

FIG. 12 depicts the characterization of Form A of Compound I-3 by thermogravimetric analysis (above) and differential scanning calorimetry (below).

FIG. 13 depicts an X-ray diffraction pattern of Form A of Compound I-4.

FIG. 14 depicts a ¹H NMR spectrum of Form A of Compound I-4 in DMSO-d6 at 400 MHz.

FIG. 15 depicts the characterization of Form A of Compound I-5 by thermogravimetric analysis (above) and differential scanning calorimetry (below).

FIG. 16 depicts an X-ray diffraction pattern of Form A of Compound I-5.

FIG. 17 depicts a ¹H NMR spectrum of Form A of Compound I-5 in DMSO-d6 at 400 MHz.

FIG. 18 depicts the characterization of Form A of Compound I-5 by thermogravimetric analysis (above) and differential scanning calorimetry (below).

FIG. 19 depicts an X-ray diffraction pattern of Form A of Compound I-6.

FIG. 20 depicts a ¹H NMR spectrum of Form A of Compound I-6 in DMSO-d6 at 400 MHz.

FIG. 21 depicts the characterization of Form A of Compound I-6 by thermogravimetric analysis (above) and differential scanning calorimetry (below).

FIG. 22 depicts an X-ray diffraction pattern of Form A of Compound I-7.

FIG. 23 depicts an X-ray diffraction pattern of Form A of Compound I-8.

FIG. 24 depicts an X-ray diffraction pattern of Form A of Compound I-9.

FIG. 25 depicts an X-ray diffraction pattern of Form A of Compound I-10.

FIG. 26 depicts an X-ray diffraction pattern of Form A of Compound I-11.

FIG. 27 depicts an X-ray diffraction pattern of Form B of Compound I-11.

### DETAILED DESCRIPTION

In some embodiments, compounds of formula (I):

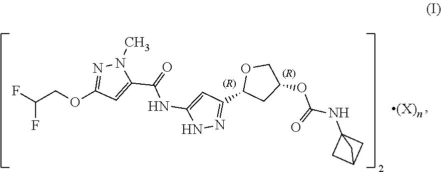

are CDK inhibitors and useful for treating disorders, diseases, and/or conditions, for example, the “CDK2-mediated” disorders, diseases, and/or conditions as described herein. In some embodiments, this disclosure provides solid forms of the compounds (e.g., as a freebase, or a salt, or a solvate) that impart characteristics such as improved aqueous solubility, stability, and ease of formulation.

### Compounds of Formula (I)

In some embodiments, provided herein is a compound of Formula (I)

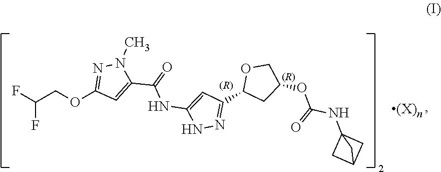

or a solvate thereof,
   wherein,
m is 1, 2, 3, 4, 5, 6, 7, 8, or 9;
n is 0, 0.5, 1, 1.5, 2, 2.5, or 3; and
X is succinic acid, urea, nicotinamide, tartaric acid, malic acid, lactic acid, maltol, L-proline, p-toluenesulfonic acid, methane sulfonic acid, benzoic acid, or 4-aminobenzoic acid.

It will be appreciated by one of ordinary skill in the art that, in some embodiments, the moiety indicated as “X” and (3R,5R)-5-(5-(3-(2,2-difluoroethoxy)-1-methyl-1H-pyrazole-5-carboxamido)-1H-pyrazol-3-yl)tetrahydrofuran-3-ylbicyclo[1.1.1]pentan-1-ylcarbamate are associated as a cocrystal to form a compound of Formula (I). It will also be appreciated that when n is 0, X is absent, indicating that the compound of Formula (I) exists as a “free form.”

It is contemplated that a compound of Formula (I) can exist in a variety of physical forms. For example, a compound of Formula (I) can be in solution, suspension, or in solid form. In certain embodiments, a compound of Formula (I) is in solid form. When a compound of Formula (I) is in solid form, said compound may be amorphous, crystalline, or a mixture thereof. Exemplary solid forms are described in more detail below.

In some embodiments, a compound of Formula (I), may be in a hydrate form. In some embodiments, a compound of Formula (I), may be in a hemi-hydrate form.

In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9.

In some embodiments, n is 0. In some embodiments, n is 1. In some embodiments, n is 2. In some embodiments, n is 3. In some embodiments, n is 0.5. In some embodiments, n is 1.5. In some embodiments, n is 2.5.

In some embodiments, X is succinic acid. In some embodiments, X is urea. In some embodiments, X is nicotinamide. In some embodiments, X is tartaric acid. In some embodiments, X is malic acid. In some embodiments, X is lactic acid. In some embodiments, X is maltol. In some embodiments, X is L-proline. In some embodiments, X is p-toluenesulfonic acid. In some embodiments, X is methane sulfonic acid. In some embodiments, X is benzoic acid. In some embodiments, X is 4-aminobenzoic acid.

In some embodiments, the present disclosure provides a form of compound I substantially free of impurities. As used herein, the term “substantially free of impurities” means that the compound or composition contains no significant amount of extraneous matter. Such extraneous matter may include different forms of compound I, residual solvents, or any other impurities that may result from the preparation of, and/or isolation of, compound I.

In some embodiments, a composition comprises a compound of Formula (I), or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising a compound of Formula (I), or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, a composition comprising a compound of Formula (I), or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of impurity compound 6 (including the free base and salts of compound 6, or solvates thereof, or solid forms thereof), wherein the percentages are based on the total weight of the composition.

In some embodiments, a composition comprises a compound of Formula (I), or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising a compound of Formula (I), or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, a composition comprising a compound of Formula (I), or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 area percent HPLC of impurity compound 6 (including the free base and salts of compound 6, or solvates thereof, or solid forms thereof) relative to the total area of the HPLC chromatogram. In some embodiments, a HPLC method is the HPLC method as described in Example 1.

The structure depicted for compound of Formula (I) is also meant to include all tautomeric forms. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

### Compound I-1

In some embodiments, a compound of Formula (I) is Compound I-1:

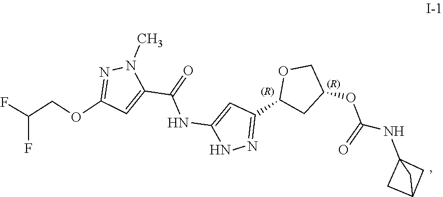

or a solvate thereof.

In another embodiment, a compound of Formula (I) is compound I-1 wherein, compound I-1 is a free form solid.

In some embodiments, Compound I-1 is an anhydrous solid. In some embodiments, Compound I-1 is an amorphous solid. In other embodiments, Compound I-1 is a crystalline solid. In some embodiments, Compound I-1 is a mixture of amorphous solid form and crystalline solid form.

In some embodiments, the present disclosure provides a form of compound I-1 substantially free of impurities. As used herein, the term “substantially free of impurities” means that the compound or composition contains no significant amount of extraneous matter. Such extraneous matter may include different forms of compound I-1, residual solvents, or any other impurities that may result from the preparation of, and/or isolation of, compound I-1.

In some embodiments, a composition comprises compound I-1, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-1, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition. In some embodiments, a composition comprising compound I-1, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of impurity compound 6 (including the free base and salts of compound 6, or solvates thereof, or solid forms thereof), wherein the percentages are based on the total weight of the composition.

In some embodiments, a composition comprises compound I-1, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-1, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram. In some embodiments, a composition comprising compound I-1, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 area percent HPLC of impurity compound 6 (including the free base and salts of compound 6, or solvates thereof, or solid forms thereof) relative to the total area of the HPLC chromatogram. In some embodiments, a HPLC method is the HPLC method as described in Example 1.

The structure depicted for compound I-1 is also meant to include all tautomeric forms of compound I-1. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In certain embodiments, compound I-1 is a crystalline solid. In other embodiments, a composition comprises a crystalline solid of compound I-1 substantially free of amorphous compound I-1. As used herein, the term “substantially free of amorphous compound I-1” means that the compound or composition contains no significant amount of amorphous compound I-1. In certain embodiments, a composition comprises at least about 95% by weight of crystalline compound I-1. In still other embodiments of the disclosure, a composition comprises at least about 99% by weight of crystalline compound I-1.

It has been found that compound I-1 can exist in a variety of solid forms. Exemplary such forms include polymorphs such as those described herein.

In some embodiments, the solid crystalline form of Compound I-1 is Form A. In some embodiments, Form A of Compound I-1 may be characterized by a powder X-ray diffraction pattern with at least two characteristic peaks, in degrees 2θ, each selected from the group consisting of about 5.2 2θ, about 18.5 2θ and about 15.6 2θ. In some embodiments, Form A of Compound I-1 may be characterized by a powder X-ray diffraction pattern with at least two characteristic peaks, in degrees 2θ, each selected from the group consisting of about 5.2 2θ, about 18.5 2θ, about 15.6 2θ, about 9.6 2θ, about 14.2 2θ, about 21.2 2θ and about 19.2 2θ. In some embodiments, Form A of Compound I-1 may be characterized by a powder X-ray diffraction pattern with at least three characteristic peaks, in degrees 2θ, each selected from the group consisting of about 5.2 2θ, about 18.5 2θ, about 15.6 2θ, about 9.6 2θ, about 14.2 2θ, about 21.2 2θ and about 19.2 2θ. In some embodiments, Form A of Compound I-1 may be characterized by a powder X-ray diffraction pattern with at least four characteristic peaks, in degrees 2θ, each selected from the group consisting of about 5.2 2θ, about 18.5 2θ, about 15.6 2θ, about 9.6 2θ, about 14.2 2θ, about 21.2 2θ and about 19.2 2θ. In some embodiments, Form A of Compound I-1 may be characterized by a powder X-ray diffraction pattern with at least five characteristic peaks, in degrees 2θ, each selected from the group consisting of about about 5.2 2θ, about 18.5 2θ, about 15.6 2θ, about 9.6 2θ, about 14.2 2θ, about 21.2 2θ and about 19.2 2θ. In some embodiments, Form A of Compound I-1 may be characterized by a powder X-ray diffraction pattern with at least six characteristic peaks, in degrees 2θ, each selected from the group consisting of about 5.2 2θ, about 18.5 2θ, about 15.6 2θ, about 9.6 2θ, about 14.2 2θ, about 21.2 2θ and about 19.2 2θ.

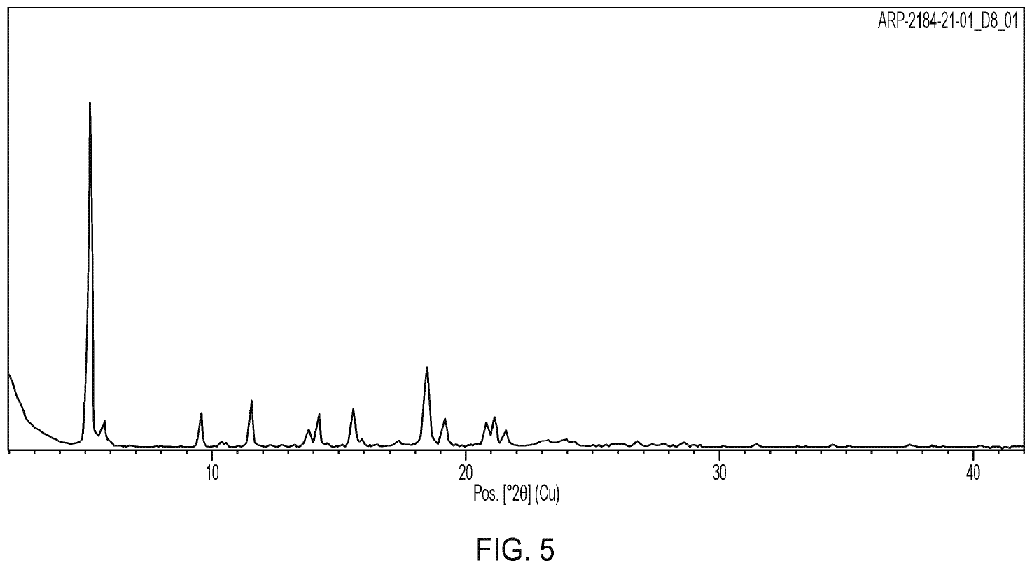

In some embodiments, Form A of Compound I-1 has an X-Ray diffraction pattern substantially similar to that depicted in FIG. 5. In some embodiments, Form A of Compound I-1 may be characterized by a powder X-ray diffraction pattern with at least two characteristic peaks, in degrees 2θ, each selected from the group consisting of the peaks listed in Table 2. In some embodiments, Form A of Compound I-1 may be characterized by a powder X-ray diffraction pattern with at least three characteristic peaks, in degrees 2θ, each selected from the group consisting of the peaks listed in Table 2. In some embodiments, Form A of Compound I-1 may be characterized by a powder X-ray diffraction pattern with at least four characteristic peaks, in degrees 2θ, each selected from the group consisting of the peaks listed in Table 2. In some embodiments, Form A of Compound I-1 may be characterized by a powder X-ray diffraction pattern with at least five characteristic peaks, in degrees 2θ, each selected from the group consisting of the peaks listed in Table 2. In some embodiments, Form A of Compound I-1 may be characterized by a powder X-ray diffraction pattern with at least six characteristic peaks, in degrees 2θ, each selected from the group consisting of the peaks listed in Table 2.

In certain embodiments, a solid crystalline form of Compound I-1 has an X-Ray diffraction pattern substantially similar to any one of the patterns depicted in FIG. 5.

As used herein, the term “about” in the context of peaks at degrees 2θ means that a peak can be the given 2θ value±0.2, or the given 2θ value±0.1, or the given value. For example, a peak of “about 23.8 2θ” means a peak can be 23.6 2θ, 23.7 2θ, 23.8 2θ, 23.9 2θ, 24.0 2θ, 23.81 2θ, 23.82 2θ, 23.83 2θ, etc.

In some embodiments, the solid crystalline form of Compound I-1 is Form B. In some embodiments, Form B of Compound I-1 may be characterized by a powder X-ray diffraction pattern with at least two characteristic peaks, in degrees 2θ, each selected from the group consisting of about 4.2 2θ, about 22.6 2θ and about 19.7 2θ. In some embodiments, Form B of Compound I-1 may be characterized by a powder X-ray diffraction pattern with at least two characteristic peaks, in degrees 2θ, each selected from the group consisting of about 4.2 2θ, about 22.6 2θ, about 19.7 2θ, about 18.8 2θ, about 23.4 2θ, about 7.3 2θ and about 11.3 2θ. In some embodiments, Form B of Compound I-1 may be characterized by a powder X-ray diffraction pattern with at least three characteristic peaks, in degrees 2θ, each selected from the group consisting of about 4.2 2θ, about 22.6 2θ, about 19.7 2θ, about 18.8 2θ, about 23.4 2θ, about 7.3 2θ and about 11.3 2θ. In some embodiments, Form B of Compound I-1 may be characterized by a powder X-ray diffraction pattern with at least four characteristic peaks, in degrees 2θ, each selected from the group consisting of about 4.2 2θ, about 22.6 2θ, about 19.7 2θ, about 18.8 2θ, about 23.4 2θ, about 7.3 2θ and about 11.3 2θ. In some embodiments, Form B of Compound I-1 may be characterized by a powder X-ray diffraction pattern with at least five characteristic peaks, in degrees 2θ, each selected from the group consisting of about about 4.2 2θ, about 22.6 2θ, about 19.7 2θ, about 18.8 2θ, about 23.4 2θ, about 7.3 2θ and about 11.3 2θ.

In some embodiments, Form B of Compound I-1 may be characterized by a powder X-ray diffraction pattern with at least six characteristic peaks, in degrees 2θ, each selected from the group consisting of about 4.2 2θ, about 22.6 2θ, about 19.7 2θ, about 18.8 2θ, about 23.4 2θ, about 7.3 2θ and about 11.3 2θ. In some embodiments, the solid crystalline form of Compound I-1 is Form B.

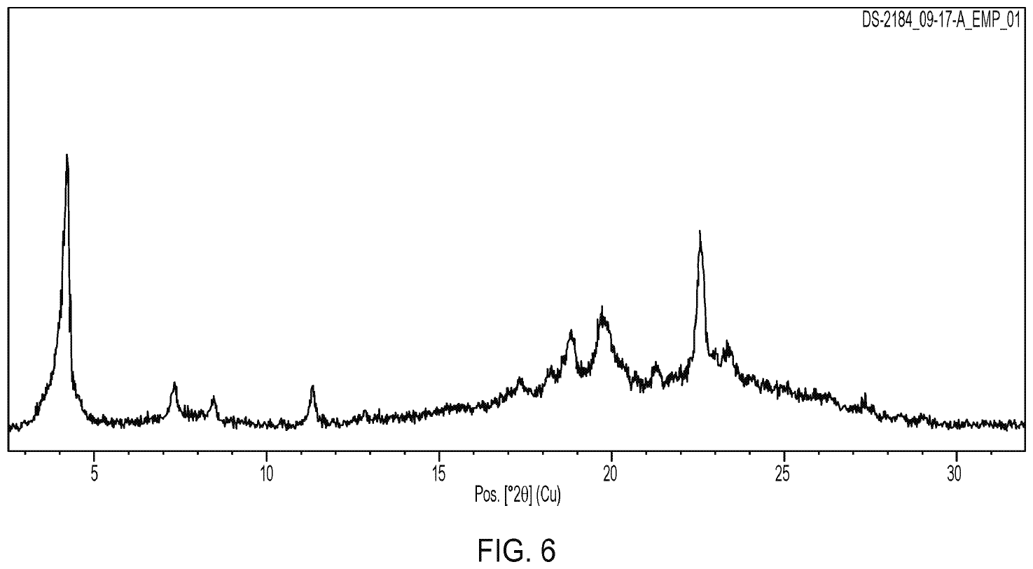

In some embodiments, Form B of compound I-1 has an X-Ray diffraction pattern substantially similar to that depicted in FIG. 6. In some embodiments, Form B of Compound I-1 may be characterized by a powder X-ray diffraction pattern with at least two characteristic peaks, in degrees 2θ, each selected from the group consisting of the peaks listed in Table 3. In some embodiments, Form B of Compound I-1 may be characterized by a powder X-ray diffraction pattern with at least three characteristic peaks, in degrees 2θ, each selected from the group consisting of the peaks listed in Table 3. In some embodiments, Form B of Compound I-1 may be characterized by a powder X-ray diffraction pattern with at least four characteristic peaks, in degrees 2θ, each selected from the group consisting of the peaks listed in Table 3. In some embodiments, Form B of Compound I-1 may be characterized by a powder X-ray diffraction pattern with at least five characteristic peaks, in degrees 2θ, each selected from the group consisting of the peaks listed in Table 3. In some embodiments, Form B of Compound I-1 may be characterized by a powder X-ray diffraction pattern with at least six characteristic peaks, in degrees 2θ, each selected from the group consisting of the peaks listed in Table 3.

In certain embodiments, a solid crystalline form of Compound I-1 has an X-Ray diffraction pattern substantially similar to any one of the patterns depicted in FIG. 6.

### Compound I-2

In some embodiments, a compound of Formula (I) is a Compound I-2:

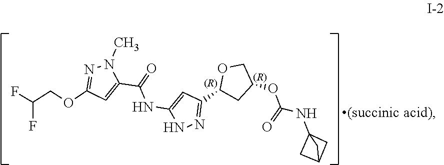

or a solvate thereof.

In another embodiment, a compound of Formula (I) is Compound I-2, which is a succinic acid cocrystal.

In some embodiments, Compound I-2 is a crystalline solid. In some embodiments, Compound I-2 is a mixture of amorphous solid form and crystalline solid form.

In some embodiments, the present disclosure provides a form of compound I-2 substantially free of impurities. As used herein, the term “substantially free of impurities” means that the compound or composition contains no significant amount of extraneous matter. Such extraneous matter may include different forms of compound I-2, residual solvents, or any other impurities that may result from the preparation of, and/or isolation of, compound I-2.

In some embodiments, a composition comprises compound I-2, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-2, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition.

In some embodiments, a composition comprises compound I-2, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-2, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram.

The structure depicted for compound I-2 is also meant to include all tautomeric forms of compound I-2. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In other embodiments, a composition comprises a crystalline solid of compound I-2 substantially free of amorphous compound I-2. As used herein, the term “substantially free of amorphous compound I-2” means that the compound or composition contains no significant amount of amorphous compound I-2. In certain embodiments, a composition comprises at least about 95% by weight of crystalline compound I-2. In certain embodiments, a composition comprises at least about 99% by weight of crystalline compound I-2.

In some embodiments, the solid crystalline form of Compound I-2 is Form A. In some embodiments, Form A of Compound I-2 may be characterized by a powder X-ray diffraction pattern with at least two characteristic peaks, in degrees 2θ, each selected from the group consisting of about 5.3 2θ, about 19.6 2θ and about 10.6 2θ. In some embodiments, Form A of Compound I-2 may be characterized by a powder X-ray diffraction pattern with at least two characteristic peaks, in degrees 2θ, each selected from the group consisting of about 5.3 2θ, about 19.6 2θ, about 10.6 2θ, about 12.2 2θ, about 18.4 2θ, about 12.4 2θ and about 20.0 2θ. In some embodiments, Form A of Compound I-2 may be characterized by a powder X-ray diffraction pattern with at least three characteristic peaks, in degrees 2θ, each selected from the group consisting of about 5.3 2θ, about 19.6 2θ, about 10.6 2θ, about 12.2 2θ, about 18.4 2θ, about 12.4 2θ and about 20.0 2θ. In some embodiments, Form A of Compound I-2 may be characterized by a powder X-ray diffraction pattern with at least four characteristic peaks, in degrees 2θ, each selected from the group consisting of about 5.3 2θ, about 19.6 2θ, about 10.6 2θ, about 12.2 2θ, about 18.4 2θ, about 12.4 2θ and about 20.0 2θ. In some embodiments, Form A of Compound I-2 may be characterized by a powder X-ray diffraction pattern with at least five characteristic peaks, in degrees 2θ, each selected from the group consisting of about about 5.3 2θ, about 19.6 2θ, about 10.6 2θ, about 12.2 2θ, about 18.4 2θ, about 12.4 2θ and about 20.0 2θ. In some embodiments, Form A of Compound I-2 may be characterized by a powder X-ray diffraction pattern with at least six characteristic peaks, in degrees 2θ, each selected from the group consisting of about 5.3 2θ, about 19.6 2θ, about 10.6 2θ, about 12.2 2θ, about 18.4 2θ, about 12.4 2θ and about 20.0 2θ.

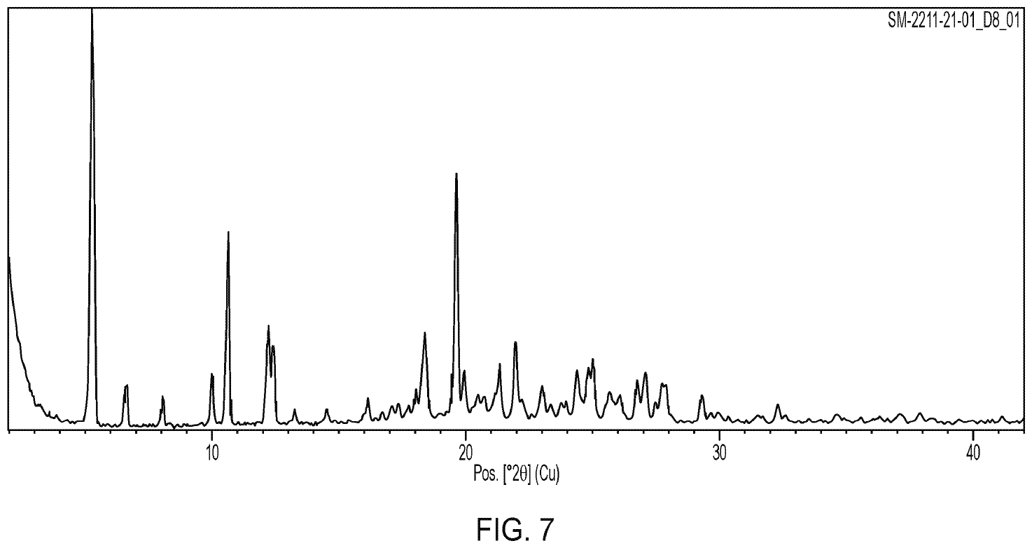

In some embodiments, Form A of Compound I-2 has an X-Ray diffraction pattern substantially similar to that depicted in FIG. 7. In some embodiments, Form A of Compound I-2 may be characterized by a powder X-ray diffraction pattern with at least two characteristic peaks, in degrees 2θ, each selected from the group consisting of the peaks listed in Table 5. In some embodiments, Form A of Compound I-2 may be characterized by a powder X-ray diffraction pattern with at least three characteristic peaks, in degrees 2θ, each selected from the group consisting of the peaks listed in Table 5. In some embodiments, Form A of Compound I-2 may be characterized by a powder X-ray diffraction pattern with at least four characteristic peaks, in degrees 2θ, each selected from the group consisting of the peaks listed in Table 5. In some embodiments, Form A of Compound I-2 may be characterized by a powder X-ray diffraction pattern with at least five characteristic peaks, in degrees 2θ, each selected from the group consisting of the peaks listed in Table 5. In some embodiments, Form A of Compound I-2 may be characterized by a powder X-ray diffraction pattern with at least six characteristic peaks, in degrees 2θ, each selected from the group consisting of the peaks listed in Table 5.

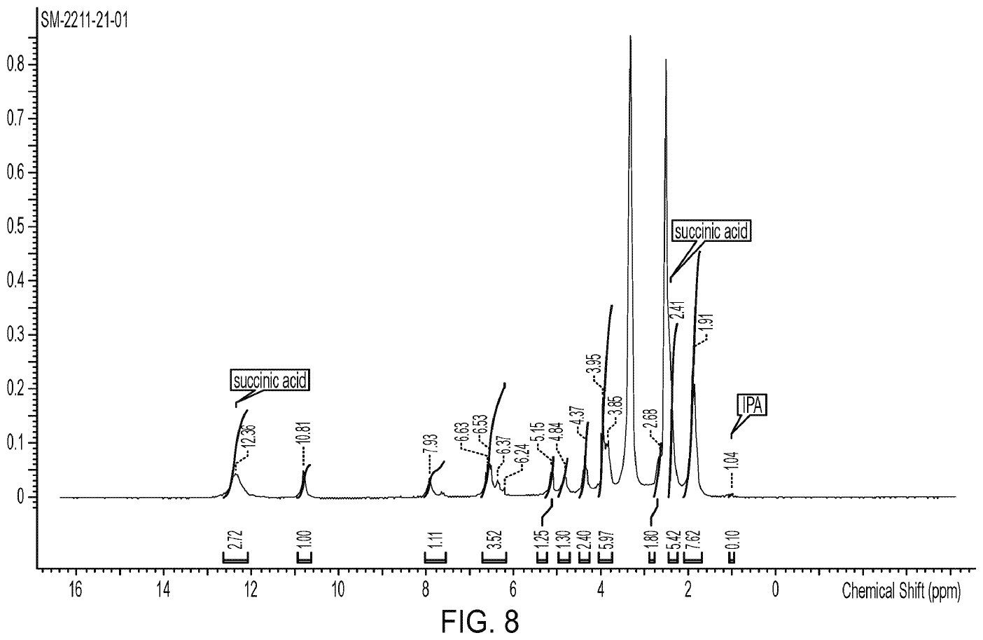

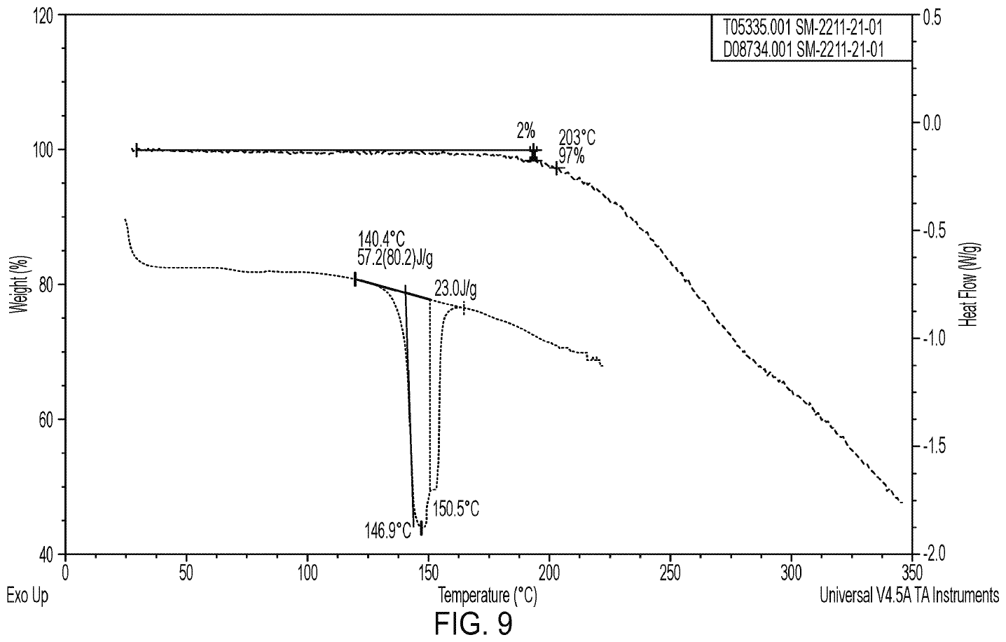

In certain embodiments, a solid crystalline form of Compound I-2 has an X-Ray diffraction pattern substantially similar to any one of the patterns depicted in FIG. 7. In certain embodiments, a solid crystalline form of compound I-2 has a ¹H NMR spectrum substantially similar to that depicted in FIG. 8. In certain embodiments, a solid crystalline form of compound I-2 has a thermogravimetric analysis pattern substantially similar to that depicted in FIG. 9. In certain embodiments, a solid crystalline form of compound I-2 has a differential scanning calorimetry pattern substantially similar to that depicted in FIG. 9. In certain embodiments, a solid crystalline form of Compound I-2 can be characterized by substantial similarity to two or more of these figures simultaneously.

### Compound I-3

In some embodiments, a compound of Formula (I) is Compound I-3:

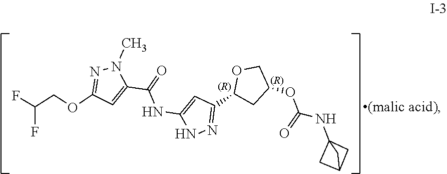

or a solvate thereof.

In another embodiment, a compound of Formula (I) is compound I-3, which is a malic acid cocrystal.

In some embodiments compound I-3 is a crystalline solid. In some embodiments, Compound I-3 is a mixture of amorphous solid form and crystalline solid form.

In some embodiments, the present disclosure provides a form of compound I-3 substantially free of impurities. As used herein, the term “substantially free of impurities” means that the compound or composition contains no significant amount of extraneous matter. Such extraneous matter may include different forms of compound I-3, residual solvents, or any other impurities that may result from the preparation of, and/or isolation of, compound I-3.

In some embodiments, a composition comprises compound I-3, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-3, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition.

In some embodiments, a composition comprises compound I-3, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-3, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram.

The structure depicted for compound I-3 is also meant to include all tautomeric forms of compound I-3. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In other embodiments, a composition comprises a crystalline solid of compound I-3 substantially free of amorphous compound I-3. As used herein, the term “substantially free of amorphous compound I-3” means that the compound or composition contains no significant amount of amorphous compound I-3. In certain embodiments, a composition comprises at least about 95% by weight of crystalline compound I-3. In certain embodiments, a composition comprises at least about 99% by weight of crystalline compound I-3.

In some embodiments, the solid crystalline form of Compound I-3 is Form A. In some embodiments, Form A of Compound I-3 may be characterized by a powder X-ray diffraction pattern with at least two characteristic peaks, in degrees 2θ, each selected from the group consisting of about 5.3 2θ, about 20.8 2θ and about 13.3 2θ. In some embodiments, Form A of Compound I-3 may be characterized by a powder X-ray diffraction pattern with at least two characteristic peaks, in degrees 2θ, each selected from the group consisting of about 5.3 2θ, about 20.8 2θ, about 13.3 2θ, about 17.6 2θ, about 18.3 2θ, about 23.6 2θ and about 20.3 2θ. In some embodiments, Form A of Compound I-3 may be characterized by a powder X-ray diffraction pattern with at least three characteristic peaks, in degrees 2θ, each selected from the group consisting of about 5.3 2θ, about 20.8 2θ, about 13.3 2θ, about 17.6 2θ, about 18.3 2θ, about 23.6 2θ and about 20.3 2θ. In some embodiments, Form A of Compound I-3 may be characterized by a powder X-ray diffraction pattern with at least four characteristic peaks, in degrees 2θ, each selected from the group consisting of about 5.3 2θ, about 20.8 2θ, about 13.3 2θ, about 17.6 2θ, about 18.3 2θ, about 23.6 2θ and about 20.3 2θ. In some embodiments, Form A of Compound I-3 may be characterized by a powder X-ray diffraction pattern with at least five characteristic peaks, in degrees 2θ, each selected from the group consisting of about about 5.3 2θ, about 20.8 2θ, about 13.3 2θ, about 17.6 2θ, about 18.3 2θ, about 23.6 2θ and about 20.3 2θ. In some embodiments, Form A of Compound I-3 may be characterized by a powder X-ray diffraction pattern with at least six characteristic peaks, in degrees 2θ, each selected from the group consisting of about 5.3 2θ, about 20.8 2θ, about 13.3 2θ, about 17.6 2θ, about 18.3 2θ, about 23.6 2θ and about 20.3 2θ.

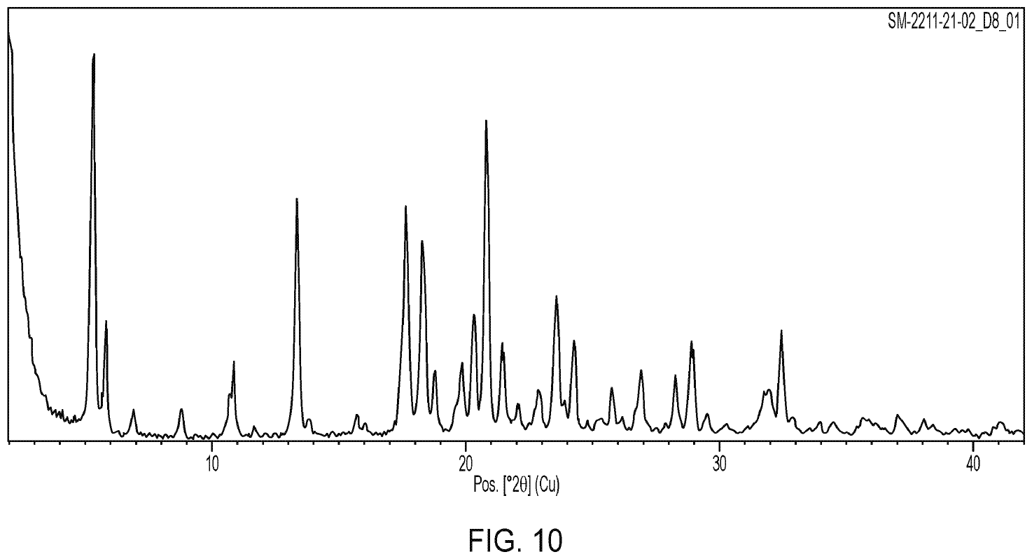

In certain embodiments, Form A of compound I-3 has an X-Ray diffraction pattern substantially similar to that depicted in FIG. 10. In some embodiments, Form A of Compound I-3 may be characterized by a powder X-ray diffraction pattern with at least two characteristic peaks, in degrees 2θ, each selected from the group consisting of the peaks listed in Table 6. In some embodiments, Form A of Compound I-3 may be characterized by a powder X-ray diffraction pattern with at least three characteristic peaks, in degrees 2θ, each selected from the group consisting of the peaks listed in Table 6. In some embodiments, Form A of Compound I-3 may be characterized by a powder X-ray diffraction pattern with at least four characteristic peaks, in degrees 2θ, each selected from the group consisting of the peaks listed in Table 6. In some embodiments, Form A of Compound I-3 may be characterized by a powder X-ray diffraction pattern with at least five characteristic peaks, in degrees 2θ, each selected from the group consisting of the peaks listed in Table 6. In some embodiments, Form A of Compound I-3 may be characterized by a powder X-ray diffraction pattern with at least six characteristic peaks, in degrees 2θ, each selected from the group consisting of the peaks listed in Table 6.

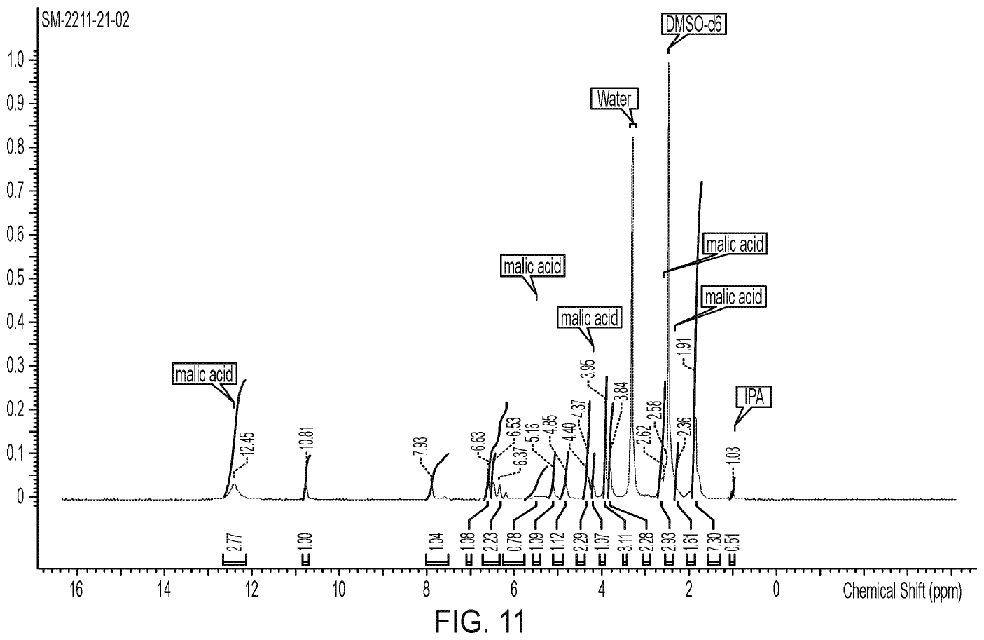

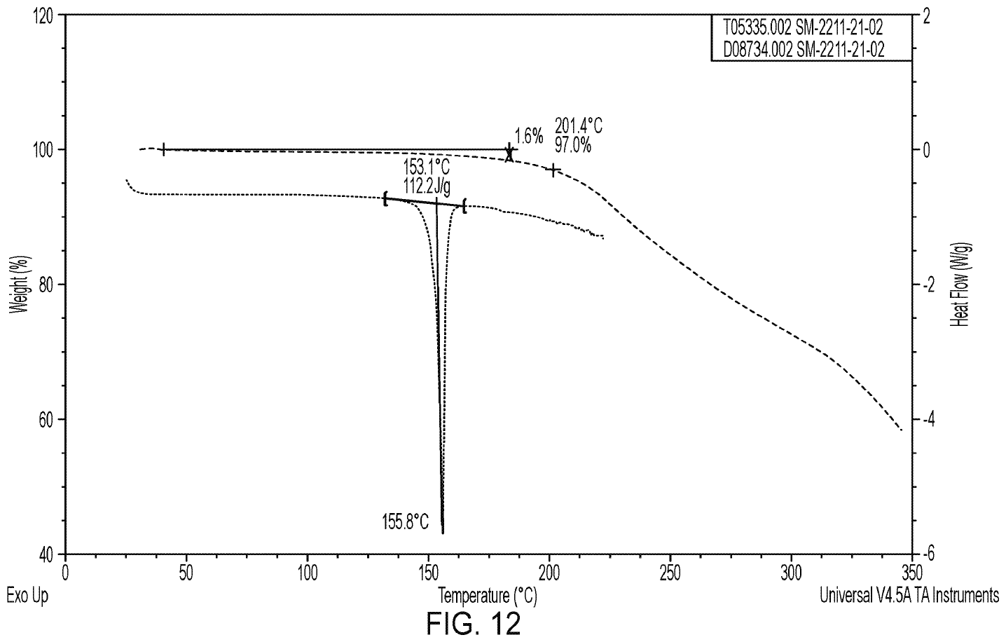

In certain embodiments, a solid crystalline form of compound I-3 has an X-Ray diffraction pattern substantially similar to any one of the patterns depicted in FIG. 10. In certain embodiments, a solid crystalline form of compound I-3 has a ¹H NMR spectrum depicted in FIG. 11. In certain embodiments, a solid crystalline form of compound I-3 has a thermogravimetric analysis pattern substantially similar to that depicted in FIG. 12. In certain embodiments, a solid crystalline form of compound I-3 has a differential scanning calorimetry pattern substantially similar to that depicted in FIG. 12. In certain embodiments, a solid crystalline form of Compound I-3 can be characterized by substantial similarity to two or more of these figures simultaneously.

### Compound I-4

In some embodiments, a compound of Formula (I) is Compound I-4:

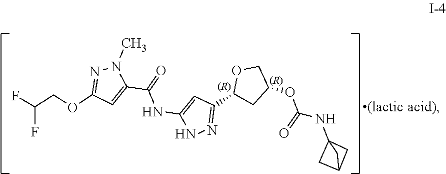

or a solvate thereof.

In another embodiment, a compound of Formula (I) is compound I-4, which is a lactic acid cocrystal.

In some embodiments compound I-4 is a crystalline solid. In some embodiments, Compound I-4 is a mixture of amorphous solid form and crystalline solid form.

In some embodiments, the present disclosure provides a form of compound I-4 substantially free of impurities. As used herein, the term “substantially free of impurities” means that the compound or composition contains no significant amount of extraneous matter. Such extraneous matter may include different forms of compound I-4, residual solvents, or any other impurities that may result from the preparation of, and/or isolation of, compound I-4.

In some embodiments, a composition comprises compound I-4, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-4, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition.

In some embodiments, a composition comprises compound I-4, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-4, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram.

The structure depicted for compound I-4 is also meant to include all tautomeric forms of compound I-4. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In other embodiments, a composition comprises a crystalline solid of compound I-4 substantially free of amorphous compound I-4. As used herein, the term “substantially free of amorphous compound I-4” means that the compound or composition contains no significant amount of amorphous compound I-4. In certain embodiments, a composition comprises at least about 95% by weight of crystalline compound I-4. In certain embodiments, a composition comprises at least about 99% by weight of crystalline compound I-4.

In some embodiments, the solid crystalline form of Compound I-4 is Form A. In some embodiments, Form A of Compound I-4 may be characterized by a powder X-ray diffraction pattern with at least two characteristic peaks, in degrees 2θ, each selected from the group consisting of about 20.4 2θ, about 5.4 2θ and about 18.8 2θ. In some embodiments, Form A of Compound I-4 may be characterized by a powder X-ray diffraction pattern with at least two characteristic peaks, in degrees 2θ, each selected from the group consisting of about 20.4 2θ, about 5.4 2θ, about 18.8 2θ, about 24.8 2θ, about 19.1 2θ, about 6.2 2θ and about 17.4 2θ. In some embodiments, Form A of Compound I-4 may be characterized by a powder X-ray diffraction pattern with at least three characteristic peaks, in degrees 2θ, each selected from the group consisting of about 20.4 2θ, about 5.4 2θ, about 18.8 2θ, about 24.8 2θ, about 19.1 2θ, about 6.2 2θ and about 17.4 2θ. In some embodiments, Form A of Compound I-4 may be characterized by a powder X-ray diffraction pattern with at least four characteristic peaks, in degrees 2θ, each selected from the group consisting of about 20.4 2θ, about 5.4 2θ, about 18.8 2θ, about 24.8 2θ, about 19.1 2θ, about 6.2 2θ and about 17.4 2θ. In some embodiments, Form A of Compound I-4 may be characterized by a powder X-ray diffraction pattern with at least five characteristic peaks, in degrees 2θ, each selected from the group consisting of about about 20.4 2θ, about 5.4 2θ, about 18.8 2θ, about 24.8 2θ, about 19.1 2θ, about 6.2 2θ and about 17.4 2θ. In some embodiments, Form A of Compound I-4 may be characterized by a powder X-ray diffraction pattern with at least six characteristic peaks, in degrees 2θ, each selected from the group consisting of about 20.4 2θ, about 5.4 2θ, about 18.8 2θ, about 24.8 2θ, about 19.1 2θ, about 6.2 2θ and about 17.4 2θ.

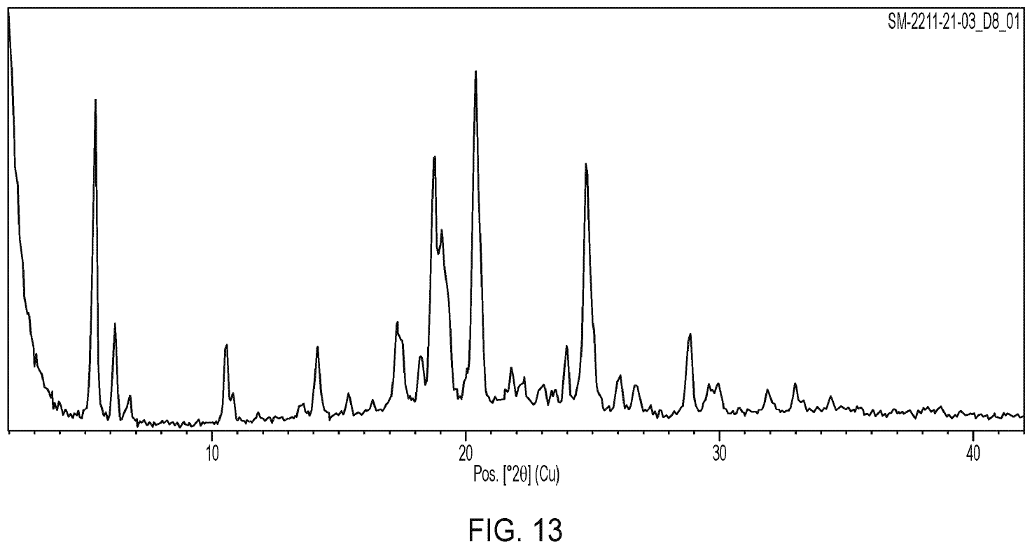

In certain embodiments, Form A of compound I-4 has an X-Ray diffraction pattern substantially similar to that depicted in FIG. 13. In some embodiments, Form A of Compound I-4 may be characterized by a powder X-ray diffraction pattern with at least two characteristic peaks, in degrees 2θ, each selected from the group consisting of the peaks listed in Table 7. In some embodiments, Form A of Compound I-4 may be characterized by a powder X-ray diffraction pattern with at least three characteristic peaks, in degrees 2θ, each selected from the group consisting of the peaks listed in Table 7. In some embodiments, Form A of Compound I-4 may be characterized by a powder X-ray diffraction pattern with at least four characteristic peaks, in degrees 2θ, each selected from the group consisting of the peaks listed in Table 7. In some embodiments, Form A of Compound I-4 may be characterized by a powder X-ray diffraction pattern with at least five characteristic peaks, in degrees 2θ, each selected from the group consisting of the peaks listed in Table 7. In some embodiments, Form A of Compound I-4 may be characterized by a powder X-ray diffraction pattern with at least six characteristic peaks, in degrees 2θ, each selected from the group consisting of the peaks listed in Table 7.

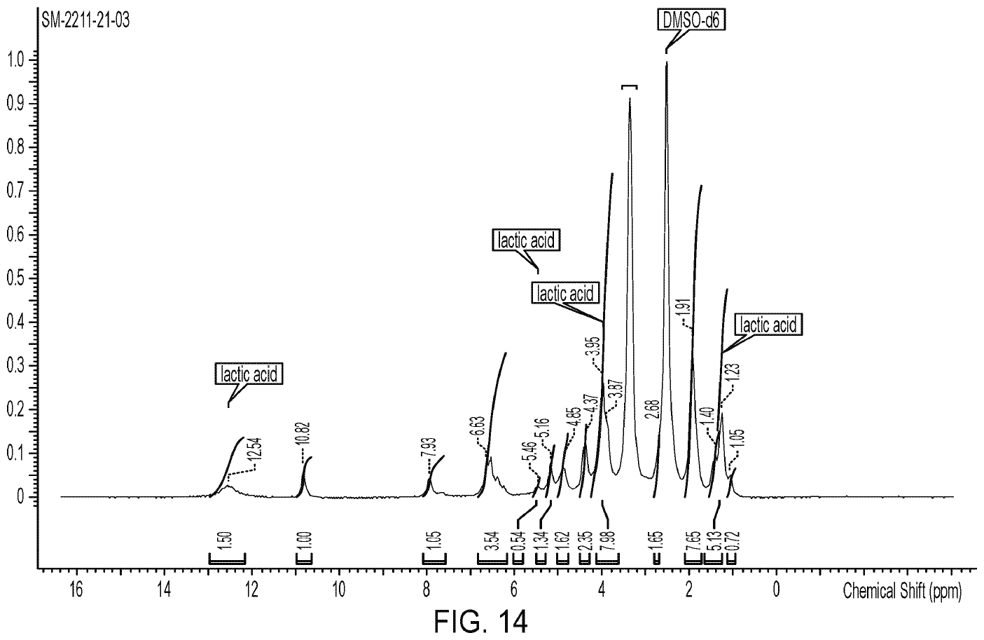

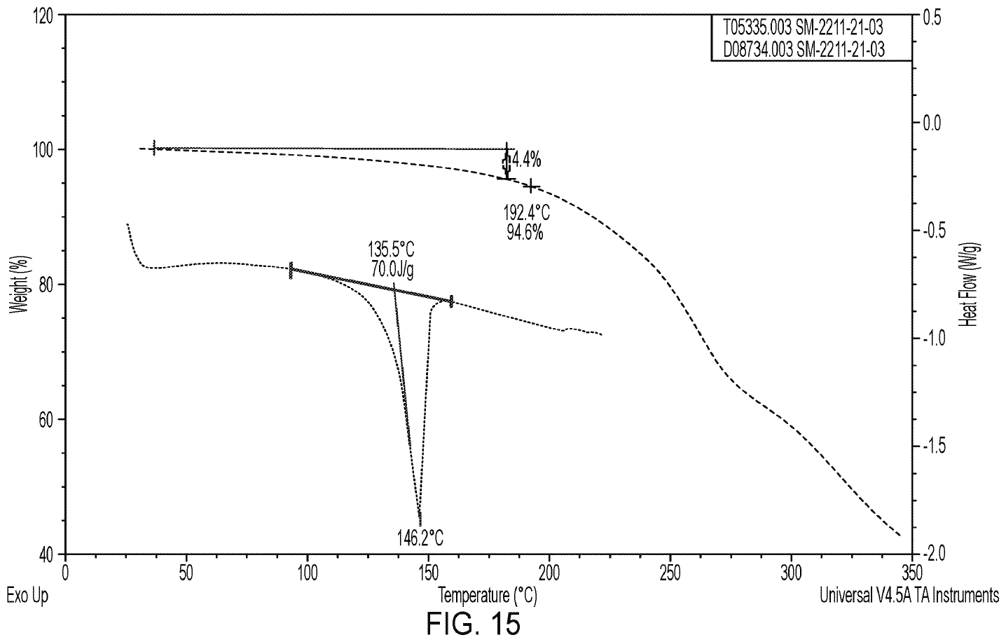

In certain embodiments, a solid crystalline form of compound I-4 has an X-Ray diffraction pattern substantially similar to any one of the patterns depicted in FIG. 13. In certain embodiments, a solid crystalline form of compound I-4 has a ¹H NMR spectrum depicted in FIG. 14. In certain embodiments, a solid crystalline form of compound I-4 has a thermogravimetric analysis pattern substantially similar to that depicted in FIG. 15. In certain embodiments, a solid crystalline form of compound I-4 has a differential scanning calorimetry pattern substantially similar to that depicted in FIG. 15. In certain embodiments, a solid crystalline form of Compound I-4 can be characterized by substantial similarity to two or more of these figures simultaneously.

### Compound I-5

In some embodiments, a compound of Formula (I) is Compound I-5:

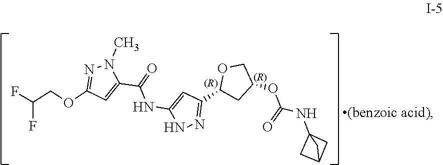

or a solvate thereof.

In another embodiment, a compound of Formula (I) is compound I-5, which is a benzoic acid cocrystal.

In some embodiments compound I-5 is a crystalline solid. In some embodiments, Compound I-5 is a mixture of amorphous solid form and crystalline solid form.

In some embodiments, the present disclosure provides a form of compound I-5 substantially free of impurities. As used herein, the term “substantially free of impurities” means that the compound or composition contains no significant amount of extraneous matter. Such extraneous matter may include different forms of compound I-5, residual solvents, or any other impurities that may result from the preparation of, and/or isolation of, compound I-5.

In some embodiments, a composition comprises compound I-5, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-5, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition.

In some embodiments, a composition comprises compound I-5, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-5, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram.

The structure depicted for compound I-5 is also meant to include all tautomeric forms of compound I-5. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In other embodiments, a composition comprises a crystalline solid of compound I-5 substantially free of amorphous compound I-5. As used herein, the term “substantially free of amorphous compound I-5” means that the compound or composition contains no significant amount of amorphous compound I-5. In certain embodiments, a composition comprises at least about 95% by weight of crystalline compound I-5. In certain embodiments, a composition comprises at least about 99% by weight of crystalline compound I-5.

In some embodiments, the solid crystalline form of Compound I-5 is Form A. In some embodiments, Form A of Compound I-5 may be characterized by a powder X-ray diffraction pattern with at least two characteristic peaks, in degrees 2θ, each selected from the group consisting of about 5.2 2θ, about 22.1 2θ and about 18.1 2θ. In some embodiments, Form A of Compound I-5 may be characterized by a powder X-ray diffraction pattern with at least two characteristic peaks, in degrees 2θ, each selected from the group consisting of about 5.2 2θ, about 22.1 2θ, about 18.1 2θ, about 18.0 2θ, about 21.3 2θ, about 17.8 2θ and about 17.2 2θ. In some embodiments, Form A of Compound I-5 may be characterized by a powder X-ray diffraction pattern with at least three characteristic peaks, in degrees 2θ, each selected from the group consisting of about 5.2 2θ, about 22.1 2θ, about 18.1 2θ, about 18.0 2θ, about 21.3 2θ, about 17.8 2θ and about 17.2 2θ. In some embodiments, Form A of Compound I-5 may be characterized by a powder X-ray diffraction pattern with at least four characteristic peaks, in degrees 2θ, each selected from the group consisting of about 5.2 2θ, about 22.1 2θ, about 18.1 2θ, about 18.0 2θ, about 21.3 2θ, about 17.8 2θ and about 17.2 2θ. In some embodiments, Form A of Compound I-5 may be characterized by a powder X-ray diffraction pattern with at least five characteristic peaks, in degrees 2θ, each selected from the group consisting of about about 5.2 2θ, about 22.1 2θ, about 18.1 2θ, about 18.0 2θ, about 21.3 2θ, about 17.8 2θ and about 17.2 2θ.

In some embodiments, Form A of Compound I-5 may be characterized by a powder X-ray diffraction pattern with at least six characteristic peaks, in degrees 2θ, each selected from the group consisting of about 5.2 2θ, about 22.1 2θ, about 18.1 2θ, about 18.0 2θ, about 21.3 2θ, about 17.8 2θ and about 17.2 2θ.

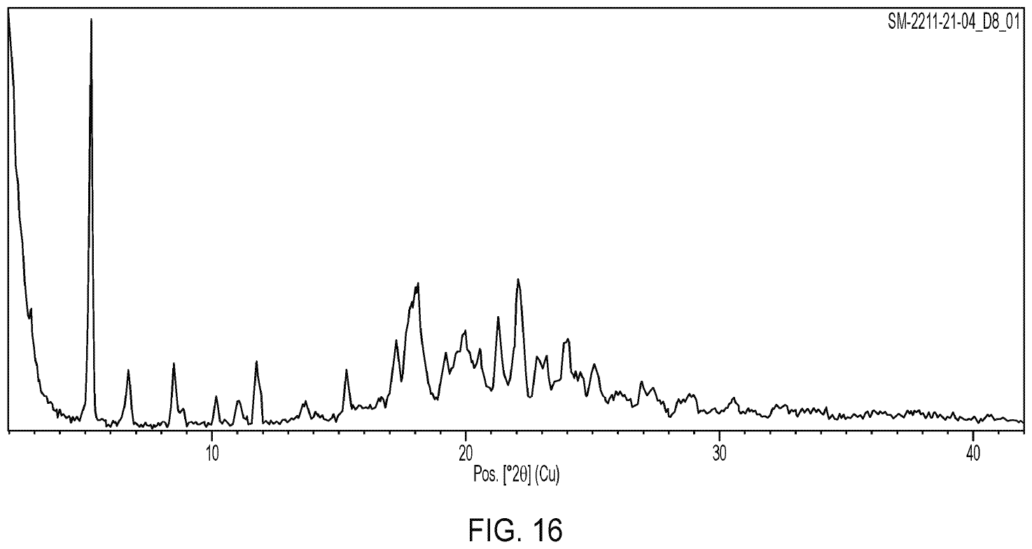

In certain embodiments, Form A of compound I-5 has an X-Ray diffraction pattern substantially similar to that depicted in FIG. 16. In some embodiments, Form A of Compound I-5 may be characterized by a powder X-ray diffraction pattern with at least two characteristic peaks, in degrees 2θ, each selected from the group consisting of the peaks listed in Table 8. In some embodiments, Form A of Compound I-5 may be characterized by a powder X-ray diffraction pattern with at least three characteristic peaks, in degrees 2θ, each selected from the group consisting of the peaks listed in Table 8. In some embodiments, Form A of Compound I-5 may be characterized by a powder X-ray diffraction pattern with at least four characteristic peaks, in degrees 2θ, each selected from the group consisting of the peaks listed in Table 8. In some embodiments, Form A of Compound I-5 may be characterized by a powder X-ray diffraction pattern with at least five characteristic peaks, in degrees 2θ, each selected from the group consisting of the peaks listed in Table 8. In some embodiments, Form A of Compound I-5 may be characterized by a powder X-ray diffraction pattern with at least six characteristic peaks, in degrees 2θ, each selected from the group consisting of the peaks listed in Table 8.

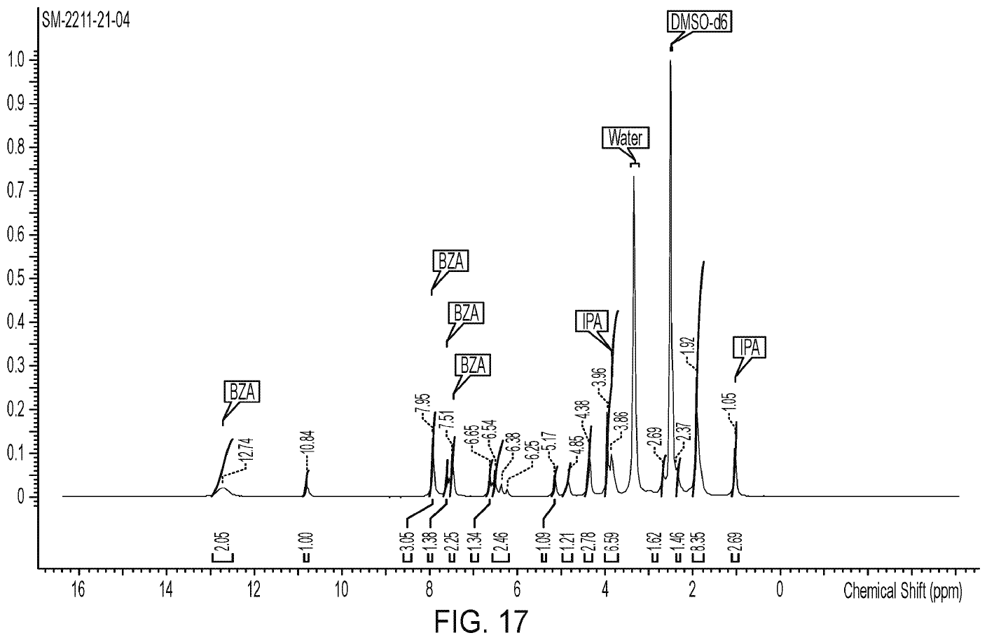

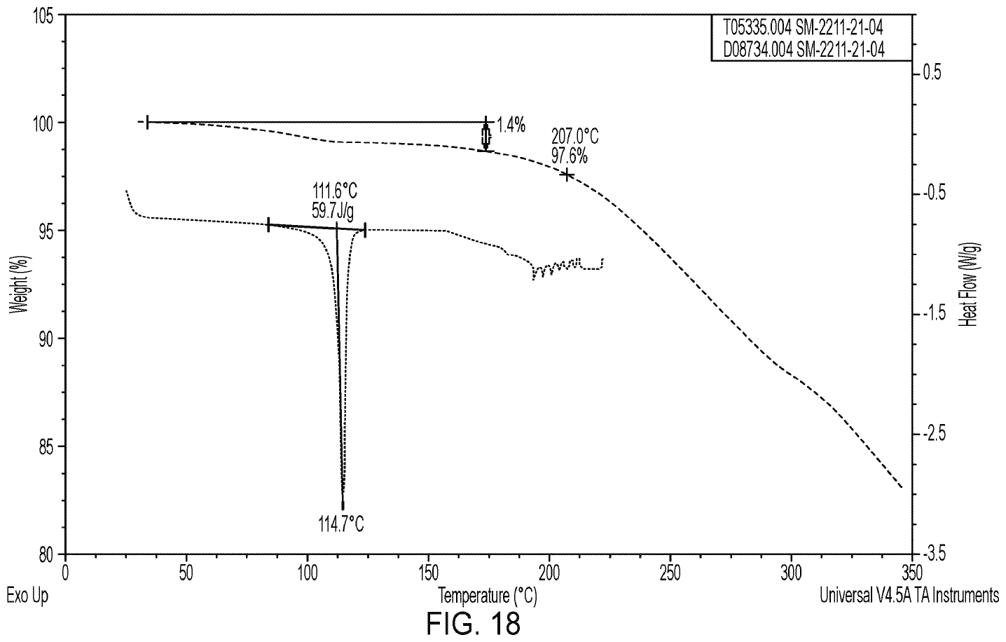

In certain embodiments, a solid crystalline form of compound I-5 has an X-Ray diffraction pattern substantially similar to any one of the patterns depicted in FIG. 16. In certain embodiments, a solid crystalline form of compound I-5 has a ¹H NMR spectrum depicted in FIG. 17. In certain embodiments, a solid crystalline form of compound I-5 has a thermogravimetric analysis pattern substantially similar to that depicted in FIG. 18. In certain embodiments, a solid crystalline form of compound I-5 has a differential scanning calorimetry pattern substantially similar to that depicted in FIG. 18. In certain embodiments, a solid crystalline form of Compound I-5 can be characterized by substantial similarity to two or more of these figures simultaneously.

### Compound I-6

In some embodiments, a compound of Formula (I) is Compound I-6:

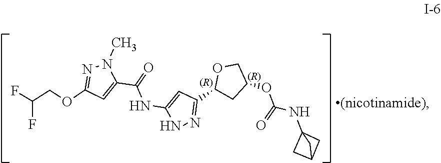

or a solvate thereof.

In another embodiment, a compound of Formula (I) is compound I-6, which is a nicotinamide cocrystal.

In some embodiments, compound I-6 is a crystalline solid. In some embodiments, Compound I-6 is a mixture of amorphous solid form and crystalline solid form.

In some embodiments, the present disclosure provides a form of compound I-6 substantially free of impurities. As used herein, the term “substantially free of impurities” means that the compound or composition contains no significant amount of extraneous matter. Such extraneous matter may include different forms of compound I-6, residual solvents, or any other impurities that may result from the preparation of, and/or isolation of, compound I-6.

In some embodiments, a composition comprises compound I-6, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-6, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition.

In some embodiments, a composition comprises compound I-6, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-6, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram.

The structure depicted for compound I-6 is also meant to include all tautomeric forms of compound I-6. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In other embodiments, a composition comprises a crystalline solid of compound I-6 substantially free of amorphous compound I-6. As used herein, the term “substantially free of amorphous compound I-6” means that the compound or composition contains no significant amount of amorphous compound I-6. In certain embodiments, a composition comprises at least about 95% by weight of crystalline compound I-6. In certain embodiments, a composition comprises at least about 99% by weight of crystalline compound I-6.

In some embodiments, the solid crystalline form of Compound I-6 is Form A. In some embodiments, Form A of Compound I-6 may be characterized by a powder X-ray diffraction pattern with at least two characteristic peaks, in degrees 2θ, each selected from the group consisting of about 4.5 2θ, about 25.6 2θ and about 22.7 2θ. In some embodiments, Form A of Compound I-6 may be characterized by a powder X-ray diffraction pattern with at least two characteristic peaks, in degrees 2θ, each selected from the group consisting of about 4.5 2θ, about 25.6 2θ, about 22.7 2θ, about 18.8 2θ, about 16.4 2θ, about 17.5 2θ and about 18.2 2θ. In some embodiments, Form A of Compound I-6 may be characterized by a powder X-ray diffraction pattern with at least three characteristic peaks, in degrees 2θ, each selected from the group consisting of about 4.5 2θ, about 25.6 2θ, about 22.7 2θ, about 18.8 2θ, about 16.4 2θ, about 17.5 2θ and about 18.2 2θ. In some embodiments, Form A of Compound I-6 may be characterized by a powder X-ray diffraction pattern with at least four characteristic peaks, in degrees 2θ, each selected from the group consisting of about 4.5 2θ, about 25.6 2θ, about 22.7 2θ, about 18.8 2θ, about 16.4 2θ, about 17.5 2θ and about 18.2 2θ. In some embodiments, Form A of Compound I-6 may be characterized by a powder X-ray diffraction pattern with at least five characteristic peaks, in degrees 2θ, each selected from the group consisting of about about 4.5 2θ, about 25.6 2θ, about 22.7 2θ, about 18.8 2θ, about 16.4 2θ, about 17.5 2θ and about 18.2 2θ. In some embodiments, Form A of Compound I-6 may be characterized by a powder X-ray diffraction pattern with at least six characteristic peaks, in degrees 2θ, each selected from the group consisting of about 4.5 2θ, about 25.6 2θ, about 22.7 2θ, about 18.8 2θ, about 16.4 2θ, about 17.5 2θ and about 18.2 2θ.

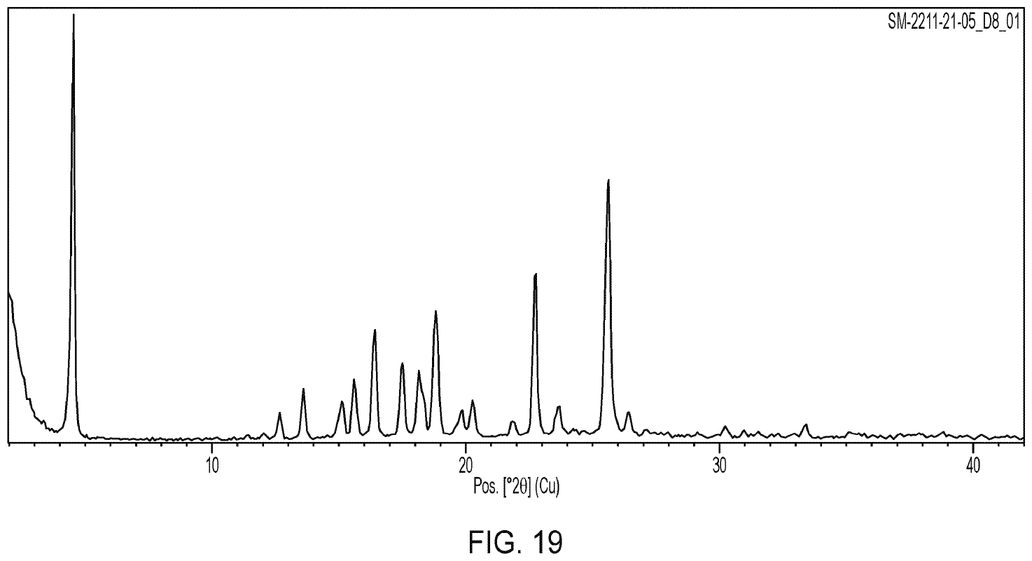

In certain embodiments, Form A of compound I-6 has an X-Ray diffraction pattern substantially similar to that depicted in FIG. 19. In some embodiments, Form A of Compound I-6 may be characterized by a powder X-ray diffraction pattern with at least two characteristic peaks, in degrees 2θ, each selected from the group consisting of the peaks listed in Table 9. In some embodiments, Form A of Compound I-6 may be characterized by a powder X-ray diffraction pattern with at least three characteristic peaks, in degrees 2θ, each selected from the group consisting of the peaks listed in Table 9. In some embodiments, Form A of Compound I-6 may be characterized by a powder X-ray diffraction pattern with at least four characteristic peaks, in degrees 2θ, each selected from the group consisting of the peaks listed in Table 9. In some embodiments, Form A of Compound I-6 may be characterized by a powder X-ray diffraction pattern with at least five characteristic peaks, in degrees 2θ, each selected from the group consisting of the peaks listed in Table 9. In some embodiments, Form A of Compound I-6 may be characterized by a powder X-ray diffraction pattern with at least six characteristic peaks, in degrees 2θ, each selected from the group consisting of the peaks listed in Table 9.

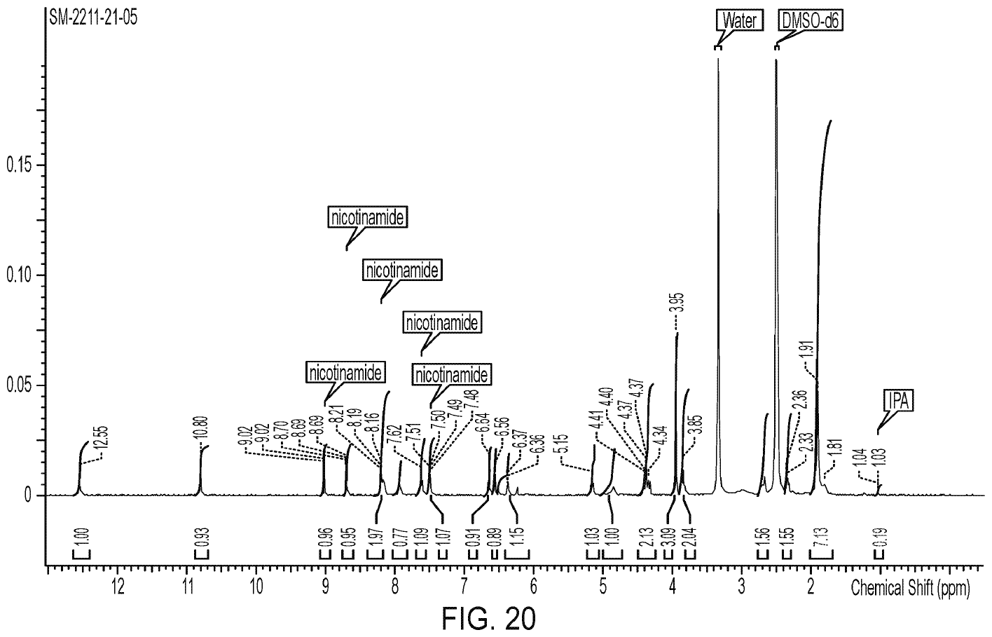

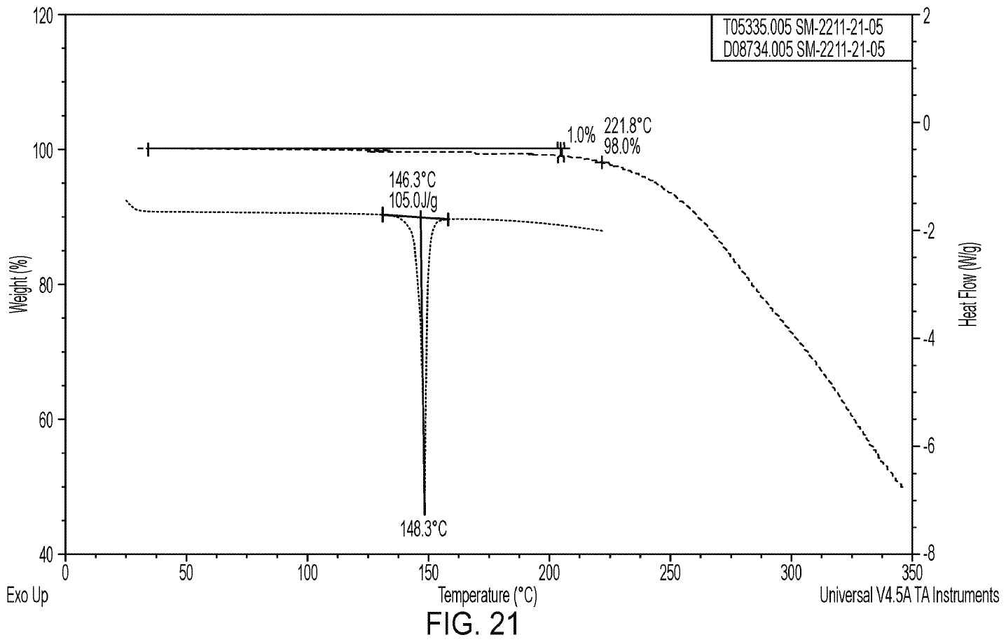

In certain embodiments, a solid crystalline form of compound I-6 has an X-Ray diffraction pattern substantially similar to any one of the patterns depicted in FIG. 19. In certain embodiments, a solid crystalline form of compound I-6 has a ¹H NMR spectrum depicted in FIG. 20. In certain embodiments, a solid crystalline form of compound I-6 has a thermogravimetric analysis pattern substantially similar to that depicted in FIG. 21. In certain embodiments, a solid crystalline form of compound I-6 has a differential scanning calorimetry pattern substantially similar to that depicted in FIG. 21. In certain embodiments, a solid crystalline form of Compound I-6 can be characterized by substantial similarity to two or more of these figures simultaneously.

### Compound I-7

In some embodiments, a compound of Formula (I) is Compound I-7:

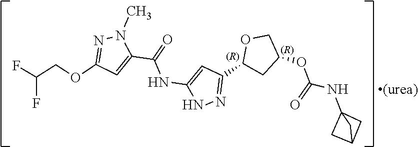

or a solvate thereof.

In another embodiment, a compound of Formula (I) is compound I-7, which is a urea cocrystal.

In some embodiments compound I-7 is a crystalline solid. In some embodiments, Compound I-7 is a mixture of amorphous solid form and crystalline solid form.

In some embodiments, the present disclosure provides a form of compound I-7 substantially free of impurities. As used herein, the term “substantially free of impurities” means that the compound or composition contains no significant amount of extraneous matter. Such extraneous matter may include different forms of compound I-7, residual solvents, or any other impurities that may result from the preparation of, and/or isolation of, compound I-7.

In some embodiments, a composition comprises compound I-7, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-7, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition.

In some embodiments, a composition comprises compound I-7, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-7, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram.

The structure depicted for compound I-7 is also meant to include all tautomeric forms of compound I-7. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In other embodiments, a composition comprises a crystalline solid of compound I-7 substantially free of amorphous compound I-7. As used herein, the term “substantially free of amorphous compound I-7” means that the compound or composition contains no significant amount of amorphous compound I-7. In certain embodiments, a composition comprises at least about 95% by weight of crystalline compound I-7. In certain embodiments, a composition comprises at least about 99% by weight of crystalline compound I-7.

In some embodiments, the solid crystalline form of Compound I-7 is Form A. In some embodiments, Form A of Compound I-7 may be characterized by a powder X-ray diffraction pattern with at least two characteristic peaks, in degrees 2θ, each selected from the group consisting of about 17.6 2θ, about 22.9 2θ and about 4.0 2θ. In some embodiments, Form A of Compound I-7 may be characterized by a powder X-ray diffraction pattern with at least two characteristic peaks, in degrees 2θ, each selected from the group consisting of about 17.6 2θ, about 22.9 2θ, about 4.0 2θ, about 17.1 2θ, about 5.8 2θ, about 18.1 2θ and about 9.0 2θ. In some embodiments, Form A of Compound I-7 may be characterized by a powder X-ray diffraction pattern with at least three characteristic peaks, in degrees 2θ, each selected from the group consisting of about 17.6 2θ, about 22.9 2θ, about 4.0 2θ, about 17.1 2θ, about 5.8 2θ, about 18.1 2θ and about 9.0 2θ. In some embodiments, Form A of Compound I-7 may be characterized by a powder X-ray diffraction pattern with at least four characteristic peaks, in degrees 2θ, each selected from the group consisting of about 17.6 2θ, about 22.9 2θ, about 4.0 2θ, about 17.1 2θ, about 5.8 2θ, about 18.1 2θ and about 9.0 2θ. In some embodiments, Form A of Compound I-7 may be characterized by a powder X-ray diffraction pattern with at least five characteristic peaks, in degrees 2θ, each selected from the group consisting of about about 17.6 2θ, about 22.9 2θ, about 4.0 2θ, about 17.1 2θ, about 5.8 2θ, about 18.1 2θ and about 9.0 2θ. In some embodiments, Form A of Compound I-7 may be characterized by a powder X-ray diffraction pattern with at least six characteristic peaks, in degrees 2θ, each selected from the group consisting of about 17.6 2θ, about 22.9 2θ, about 4.0 2θ, about 17.1 2θ, about 5.8 2θ, about 18.1 2θ and about 9.0 2θ.

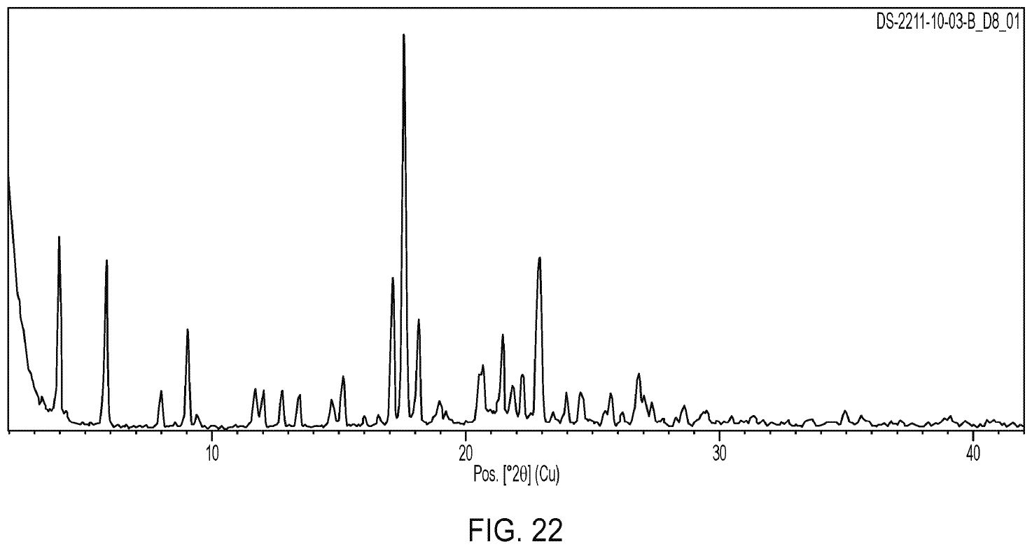

In certain embodiments, Form A of compound I-7 has an X-Ray diffraction pattern substantially similar to that depicted in FIG. 22. In some embodiments, Form A of Compound I-7 may be characterized by a powder X-ray diffraction pattern with at least two characteristic peaks, in degrees 2θ, each selected from the group consisting of the peaks listed in Table 10. In some embodiments, Form A of Compound I-7 may be characterized by a powder X-ray diffraction pattern with at least three characteristic peaks, in degrees 2θ, each selected from the group consisting of the peaks listed in Table 10. In some embodiments, Form A of Compound I-7 may be characterized by a powder X-ray diffraction pattern with at least four characteristic peaks, in degrees 2θ, each selected from the group consisting of the peaks listed in Table 10. In some embodiments, Form A of Compound I-7 may be characterized by a powder X-ray diffraction pattern with at least five characteristic peaks, in degrees 2θ, each selected from the group consisting of the peaks listed in Table 10. In some embodiments, Form A of Compound I-7 may be characterized by a powder X-ray diffraction pattern with at least six characteristic peaks, in degrees 2θ, each selected from the group consisting of the peaks listed in Table 10.

In certain embodiments, a solid crystalline form of compound I-7 has an X-Ray diffraction pattern substantially similar to any one of the patterns depicted in FIG. 22.

### Compound I-8

In some embodiments, a compound of Formula (I) is Compound I-8:

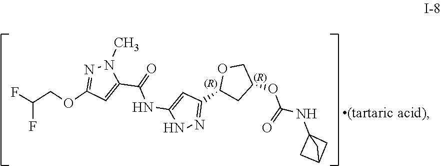

or a solvate thereof.

In another embodiment, a compound of Formula (I) is compound I-8, which is a tartaric acid cocrystal. In some embodiments, compound I-8 is an L-tartaric acid cocrystal.

In some embodiments compound I-8 is a crystalline solid. In some embodiments, Compound I-8 is a mixture of amorphous solid form and crystalline solid form.

In some embodiments, the present disclosure provides a form of compound I-8 substantially free of impurities. As used herein, the term “substantially free of impurities” means that the compound or composition contains no significant amount of extraneous matter. Such extraneous matter may include different forms of compound I-8, residual solvents, or any other impurities that may result from the preparation of, and/or isolation of, compound I-8.

In some embodiments, a composition comprises compound I-8, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-8, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition.

In some embodiments, a composition comprises compound I-8, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-8, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram.

The structure depicted for compound I-8 is also meant to include all tautomeric forms of compound I-8. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In other embodiments, a composition comprises a crystalline solid of compound I-8 substantially free of amorphous compound I-8. As used herein, the term “substantially free of amorphous compound I-8” means that the compound or composition contains no significant amount of amorphous compound I-8. In certain embodiments, a composition comprises at least about 95% by weight of crystalline compound I-8. In certain embodiments, a composition comprises at least about 99% by weight of crystalline compound I-8.

In some embodiments, the solid crystalline form of Compound I-8 is Form A. In some embodiments, Form A of Compound I-8 may be characterized by a powder X-ray diffraction pattern with at least two characteristic peaks, in degrees 2θ, each selected from the group consisting of about 5.3 2θ, about 18.8 2θ and about 22.9 2θ. In some embodiments, Form A of Compound I-8 may be characterized by a powder X-ray diffraction pattern with at least two characteristic peaks, in degrees 2θ, each selected from the group consisting of about 5.3 2θ, about 18.8 2θ, about 22.9 2θ, about 10.6 2θ, about 24.9 2θ, about 17.2 2θ and about 18.6 2θ. In some embodiments, Form A of Compound I-8 may be characterized by a powder X-ray diffraction pattern with at least three characteristic peaks, in degrees 2θ, each selected from the group consisting of about 5.3 2θ, about 18.8 2θ, about 22.9 2θ, about 10.6 2θ, about 24.9 2θ, about 17.2 2θ and about 18.6 2θ. In some embodiments, Form A of Compound I-8 may be characterized by a powder X-ray diffraction pattern with at least four characteristic peaks, in degrees 2θ, each selected from the group consisting of about 5.3 2θ, about 18.8 2θ, about 22.9 2θ, about 10.6 2θ, about 24.9 2θ, about 17.2 2θ and about 18.6 2θ. In some embodiments, Form A of Compound I-8 may be characterized by a powder X-ray diffraction pattern with at least five characteristic peaks, in degrees 2θ, each selected from the group consisting of about about 5.3 2θ, about 18.8 2θ, about 22.9 2θ, about 10.6 2θ, about 24.9 2θ, about 17.2 2θ and about 18.6 2θ. In some embodiments, Form A of Compound I-8 may be characterized by a powder X-ray diffraction pattern with at least six characteristic peaks, in degrees 2θ, each selected from the group consisting of about 5.3 2θ, about 18.8 2θ, about 22.9 2θ, about 10.6 2θ, about 24.9 2θ, about 17.2 2θ and about 18.6 2θ.

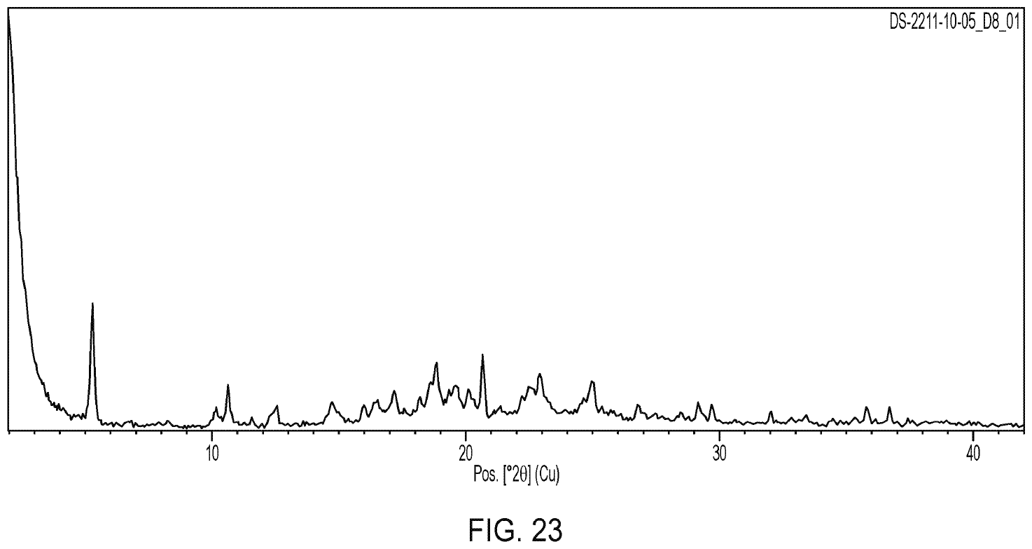

In certain embodiments, Form A of compound I-8 has an X-Ray diffraction pattern substantially similar to that depicted in FIG. 23. In some embodiments, Form A of Compound I-8 may be characterized by a powder X-ray diffraction pattern with at least two characteristic peaks, in degrees 2θ, each selected from the group consisting of the peaks listed in Table 11. In some embodiments, Form A of Compound I-8 may be characterized by a powder X-ray diffraction pattern with at least three characteristic peaks, in degrees 2θ, each selected from the group consisting of the peaks listed in Table 11. In some embodiments, Form A of Compound I-8 may be characterized by a powder X-ray diffraction pattern with at least four characteristic peaks, in degrees 2θ, each selected from the group consisting of the peaks listed in Table 11. In some embodiments, Form A of Compound I-8 may be characterized by a powder X-ray diffraction pattern with at least five characteristic peaks, in degrees 2θ, each selected from the group consisting of the peaks listed in Table 11. In some embodiments, Form A of Compound I-8 may be characterized by a powder X-ray diffraction pattern with at least six characteristic peaks, in degrees 2θ, each selected from the group consisting of the peaks listed in Table 11.

In certain embodiments, a solid crystalline form of compound I-8 has an X-Ray diffraction pattern substantially similar to any one of the patterns depicted in FIG. 23.

### Compound I-9

In some embodiments, a compound of Formula (I) is Compound I-9:

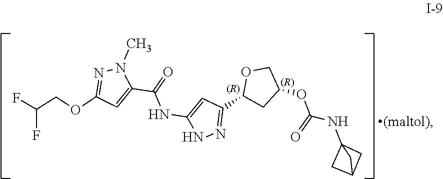

or a solvate thereof.

In another embodiment, a compound of Formula (I) is compound I-9, which is a maltol cocrystal.

In some embodiments compound I-9 is a crystalline solid. In some embodiments, Compound I-9 is a mixture of amorphous solid form and crystalline solid form.

In some embodiments, the present disclosure provides a form of compound I-9 substantially free of impurities. As used herein, the term “substantially free of impurities” means that the compound or composition contains no significant amount of extraneous matter. Such extraneous matter may include different forms of compound I-9, residual solvents, or any other impurities that may result from the preparation of, and/or isolation of, compound I-9.

In some embodiments, a composition comprises compound I-9, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-9, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition.

In some embodiments, a composition comprises compound I-9, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-9, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram.

The structure depicted for compound I-9 is also meant to include all tautomeric forms of compound I-9. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In other embodiments, a composition comprises a crystalline solid of compound I-9 substantially free of amorphous compound I-9. As used herein, the term “substantially free of amorphous compound I-9” means that the compound or composition contains no significant amount of amorphous compound I-9. In certain embodiments, a composition comprises at least about 95% by weight of crystalline compound I-9. In certain embodiments, a composition comprises at least about 99% by weight of crystalline compound I-9.

In some embodiments, the solid crystalline form of Compound I-9 is Form A. In some embodiments, Form A of Compound I-9 may be characterized by a powder X-ray diffraction pattern with at least two characteristic peaks, in degrees 2θ, each selected from the group consisting of about 17.7 2θ, about 20.1 2θ and about 24.1 2θ. In some embodiments, Form A of Compound I-9 may be characterized by a powder X-ray diffraction pattern with at least two characteristic peaks, in degrees 2θ, each selected from the group consisting of about 17.7 2θ, about 20.1 2θ, about 24.1 2θ, about 23.5 2θ, about 13.3 2θ, about 18.2 2θ and about 25.7 2θ. In some embodiments, Form A of Compound I-9 may be characterized by a powder X-ray diffraction pattern with at least three characteristic peaks, in degrees 2θ, each selected from the group consisting of about 17.7 2θ, about 20.1 2θ, about 24.1 2θ, about 23.5 2θ, about 13.3 2θ, about 18.2 2θ and about 25.7 2θ. In some embodiments, Form A of Compound I-9 may be characterized by a powder X-ray diffraction pattern with at least four characteristic peaks, in degrees 2θ, each selected from the group consisting of about 17.7 2θ, about 20.1 2θ, about 24.1 2θ, about 23.5 2θ, about 13.3 2θ, about 18.2 2θ and about 25.7 2θ. In some embodiments, Form A of Compound I-9 may be characterized by a powder X-ray diffraction pattern with at least five characteristic peaks, in degrees 2θ, each selected from the group consisting of about 17.7 2θ, about 20.1 2θ, about 24.1 2θ, about 23.5 2θ, about 13.3 2θ, about 18.2 2θ and about 25.7 2θ. In some embodiments, Form A of Compound I-9 may be characterized by a powder X-ray diffraction pattern with at least six characteristic peaks, in degrees 2θ, each selected from the group consisting of about 17.7 2θ, about 20.1 2θ, about 24.1 2θ, about 23.5 2θ, about 13.3 2θ, about 18.2 2θ and about 25.7 2θ.

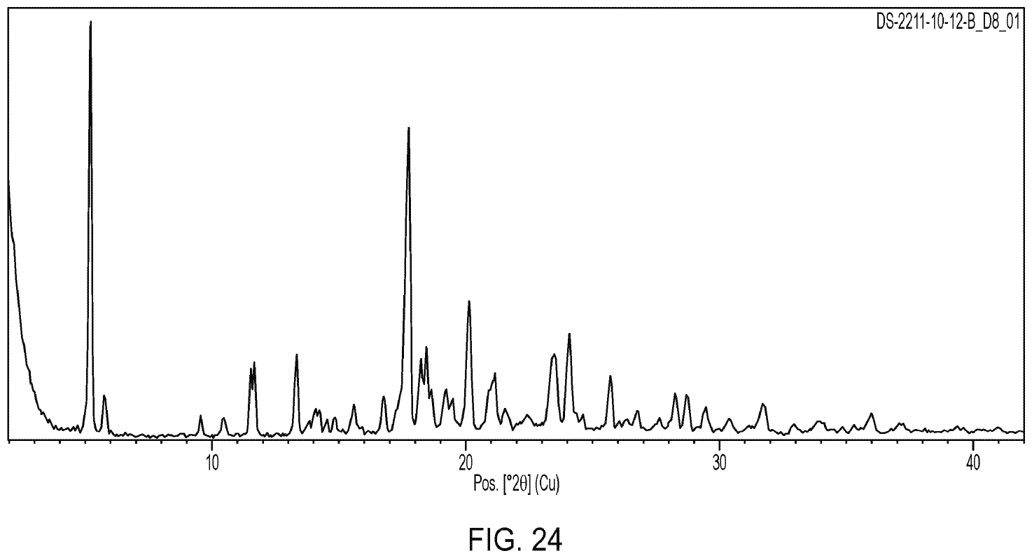

In certain embodiments, Form A of compound I-9 has an X-Ray diffraction pattern substantially similar to that depicted in FIG. 24. In some embodiments, Form A of Compound I-9 may be characterized by a powder X-ray diffraction pattern with at least two characteristic peaks, in degrees 2θ, each selected from the group consisting of the peaks listed in Table 12. In some embodiments, Form A of Compound I-9 may be characterized by a powder X-ray diffraction pattern with at least three characteristic peaks, in degrees 2θ, each selected from the group consisting of the peaks listed in Table 12. In some embodiments, Form A of Compound I-9 may be characterized by a powder X-ray diffraction pattern with at least four characteristic peaks, in degrees 2θ, each selected from the group consisting of the peaks listed in Table 12. In some embodiments, Form A of Compound I-9 may be characterized by a powder X-ray diffraction pattern with at least five characteristic peaks, in degrees 2θ, each selected from the group consisting of the peaks listed in Table 12. In some embodiments, Form A of Compound I-9 may be characterized by a powder X-ray diffraction pattern with at least six characteristic peaks, in degrees 2θ, each selected from the group consisting of the peaks listed in Table 12.

In certain embodiments, a solid crystalline form of compound I-9 has an X-Ray diffraction pattern substantially similar to any one of the patterns depicted in FIG. 24.

### Compound I-10

In some embodiments, a compound of Formula (I) is Compound I-10:

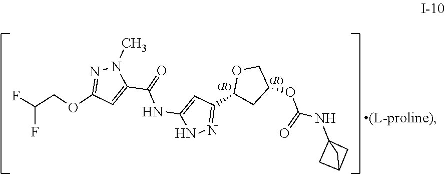

or a solvate thereof.

In another embodiment, a compound of Formula (I) is compound I-10, which is a L-proline cocrystal.

In some embodiments compound I-10 is a crystalline solid. In some embodiments, Compound I-10 is a mixture of amorphous solid form and crystalline solid form.

In some embodiments, the present disclosure provides a form of compound I-10 substantially free of impurities. As used herein, the term “substantially free of impurities” means that the compound or composition contains no significant amount of extraneous matter. Such extraneous matter may include different forms of compound I-10, residual solvents, or any other impurities that may result from the preparation of, and/or isolation of, compound I-10.

In some embodiments, a composition comprises compound I-10, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-10, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition.

In some embodiments, a composition comprises compound I-10, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-10, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram.

The structure depicted for compound I-10 is also meant to include all tautomeric forms of compound I-10. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In other embodiments, a composition comprises a crystalline solid of compound I-10 substantially free of amorphous compound I-10. As used herein, the term “substantially free of amorphous compound I-10” means that the compound or composition contains no significant amount of amorphous compound I-10. In certain embodiments, a composition comprises at least about 95% by weight of crystalline compound I-10. In certain embodiments, a composition comprises at least about 99% by weight of crystalline compound I-10.

In some embodiments, the solid crystalline form of Compound I-10 is Form A. In some embodiments, Form A of Compound I-10 may be characterized by a powder X-ray diffraction pattern with at least two characteristic peaks, in degrees 2θ, each selected from the group consisting of about 7.1 2θ, about 10.7 2θ and about 6.2 2θ. In some embodiments, Form A of Compound I-10 may be characterized by a powder X-ray diffraction pattern with at least two characteristic peaks, in degrees 2θ, each selected from the group consisting of about 7.1 2θ, about 10.7 2θ, about 6.2 2θ, about 20.1 2θ, about 18.2 2θ, about 21.5 2θ and about 21.8 2θ. In some embodiments, Form A of Compound I-10 may be characterized by a powder X-ray diffraction pattern with at least three characteristic peaks, in degrees 2θ, each selected from the group consisting of about 7.1 2θ, about 10.7 2θ, about 6.2 2θ, about 20.1 2θ, about 18.2 2θ, about 21.5 2θ and about 21.8 2θ. In some embodiments, Form A of Compound I-10 may be characterized by a powder X-ray diffraction pattern with at least four characteristic peaks, in degrees 2θ, each selected from the group consisting of about 7.1 2θ, about 10.7 2θ, about 6.2 2θ, about 20.1 2θ, about 18.2 2θ, about 21.5 2θ and about 21.8 2θ. In some embodiments, Form A of Compound I-10 may be characterized by a powder X-ray diffraction pattern with at least five characteristic peaks, in degrees 2θ, each selected from the group consisting of about 7.1 2θ, about 10.7 2θ, about 6.2 2θ, about 20.1 2θ, about 18.2 2θ, about 21.5 2θ and about 21.8 2θ. In some embodiments, Form A of Compound I-10 may be characterized by a powder X-ray diffraction pattern with at least six characteristic peaks, in degrees 2θ, each selected from the group consisting of about 7.1 2θ, about 10.7 2θ, about 6.2 2θ, about 20.1 2θ, about 18.2 2θ, about 21.5 2θ and about 21.8 2θ.

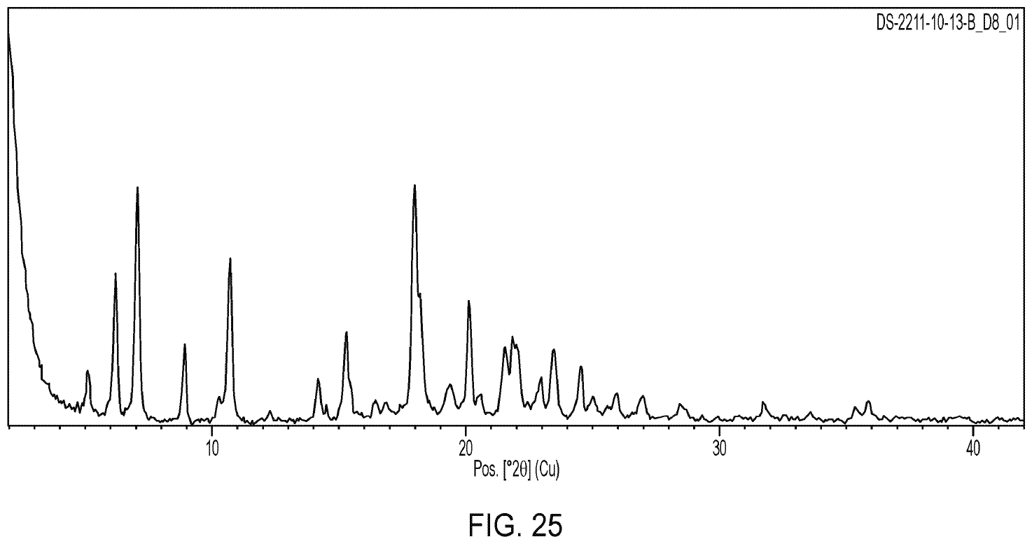

In certain embodiments, Form A of compound I-10 has an X-Ray diffraction pattern substantially similar to that depicted in FIG. 25. In some embodiments, Form A of Compound I-10 may be characterized by a powder X-ray diffraction pattern with at least two characteristic peaks, in degrees 2θ, each selected from the group consisting of the peaks listed in Table 13. In some embodiments, Form A of Compound I-10 may be characterized by a powder X-ray diffraction pattern with at least three characteristic peaks, in degrees 2θ, each selected from the group consisting of the peaks listed in Table 13. In some embodiments, Form A of Compound I-10 may be characterized by a powder X-ray diffraction pattern with at least four characteristic peaks, in degrees 2θ, each selected from the group consisting of the peaks listed in Table 13. In some embodiments, Form A of Compound I-10 may be characterized by a powder X-ray diffraction pattern with at least five characteristic peaks, in degrees 2θ, each selected from the group consisting of the peaks listed in Table 13. In some embodiments, Form A of Compound I-10 may be characterized by a powder X-ray diffraction pattern with at least six characteristic peaks, in degrees 2θ, each selected from the group consisting of the peaks listed in Table 13.

In certain embodiments, a solid crystalline form of compound I-10 has an X-Ray diffraction pattern substantially similar to any one of the patterns depicted in FIG. 25.

### Compound I-11

In some embodiments, a compound of Formula (I) is Compound I-11:

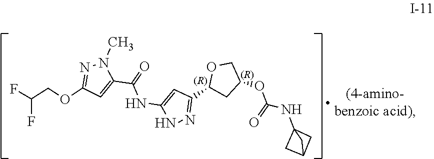

or a solvate thereof.

In another embodiment, a compound of Formula (I) is compound I-11, which is a 4-aminobenzoic acid cocrystal.

In some embodiments compound I-11 is a crystalline solid. In some embodiments, Compound I-11 is a mixture of amorphous solid form and crystalline solid form.

In some embodiments, the present disclosure provides a form of compound I-11 substantially free of impurities. As used herein, the term “substantially free of impurities” means that the compound or composition contains no significant amount of extraneous matter. Such extraneous matter may include different forms of compound I-11, residual solvents, or any other impurities that may result from the preparation of, and/or isolation of, compound I-11.

In some embodiments, a composition comprises compound I-11, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 weight percent where the percentages are based on the total weight of the composition. In some embodiments, a composition comprising compound I-11, or a solvate thereof, or a crystalline form thereof, contains no more than about 0.40, no more than about 0.35, no more than about 0.3, no more than about 0.25, no more than about 0.2, no more than about 0.15, no more than about 0.10, or no more than about 0.05 weight percent of any single impurity wherein the percentages are based on the total weight of the composition.

In some embodiments, a composition comprises compound I-11, or a solvate thereof, or a crystalline form thereof, in an amount of at least about 95, 95.5, 96, 96.5, 97, 97.5, 98.0, 98.5, 99, 99.1, 99.2, 99.3, 99.4, 99.5, 99.6, 99.7, 99.8, 99.9 area percent by HPLC relative to the total area of the HPLC chromatogram. In some embodiments, a composition comprising compound I-11, or a solvate thereof, or a crystalline form thereof, contains no more than about 0.4, no more than about 0.35, no more than about 0.3, no more than about 0.25, no more than about 0.2, no more than about 0.15, no more than about 0.10, or no more than about 0.05 area percent HPLC of any single impurity relative to the total area of the HPLC chromatogram.

The structure depicted for compound I-11 is also meant to include all tautomeric forms of compound I-11. Additionally, structures depicted here are also meant to include compounds that differ only in the presence of one or more isotopically enriched atoms. For example, compounds having the present structure except for the replacement of hydrogen by deuterium or tritium, or the replacement of a carbon by a ¹³C- or ¹⁴C-enriched carbon are within the scope of this disclosure.

In other embodiments, a composition comprises a crystalline solid of compound I-11 substantially free of amorphous compound I-11. As used herein, the term “substantially free of amorphous compound I-11” means that the compound or composition contains no significant amount of amorphous compound I-11. In certain embodiments, a composition comprises at least about 95% by weight of crystalline compound I-11. In certain embodiments, a composition comprises at least about 99% by weight of crystalline compound I-11.

In some embodiments, the solid crystalline form of Compound I-11 is Form A. In some embodiments, Form A of Compound I-11 may be characterized by a powder X-ray diffraction pattern with at least two characteristic peaks, in degrees 2θ, each selected from the group consisting of about 20.8 2θ, about 20.1 2θ and about 5.4 2θ. In some embodiments, Form A of Compound I-11 may be characterized by a powder X-ray diffraction pattern with at least two characteristic peaks, in degrees 2θ, each selected from the group consisting of about 20.8 2θ, about 20.1 2θ, about 5.4 2θ, about 20.1 2θ, about 24.0 2θ, about 24.7 2θ and about 4.9 2θ. In some embodiments, Form A of Compound I-11 may be characterized by a powder X-ray diffraction pattern with at least three characteristic peaks, in degrees 2θ, each selected from the group consisting of about 20.8 2θ, about 20.1 2θ, about 5.4 2θ, about 20.1 2θ, about 24.0 2θ, about 24.7 2θ and about 4.9 2θ. In some embodiments, Form A of Compound I-11 may be characterized by a powder X-ray diffraction pattern with at least four characteristic peaks, in degrees 2θ, each selected from the group consisting of about 20.8 2θ, about 20.1 2θ, about 5.4 2θ, about 20.1 2θ, about 24.0 2θ, about 24.7 2θ and about 4.9 2θ. In some embodiments, Form A of Compound I-11 may be characterized by a powder X-ray diffraction pattern with at least five characteristic peaks, in degrees 2θ, each selected from the group consisting of about 20.8 2θ, about 20.1 2θ, about 5.4 2θ, about 20.1 2θ, about 24.0 2θ, about 24.7 2θ and about 4.9 2θ. In some embodiments, Form A of Compound I-11 may be characterized by a powder X-ray diffraction pattern with at least six characteristic peaks, in degrees 2θ, each selected from the group consisting of about 7.1 2θ, about 10.7 2θ, about 6.2 2θ, about 20.1 2θ, about 18.2 2θ, about 21.5 2θ and about 21.8 2θ.

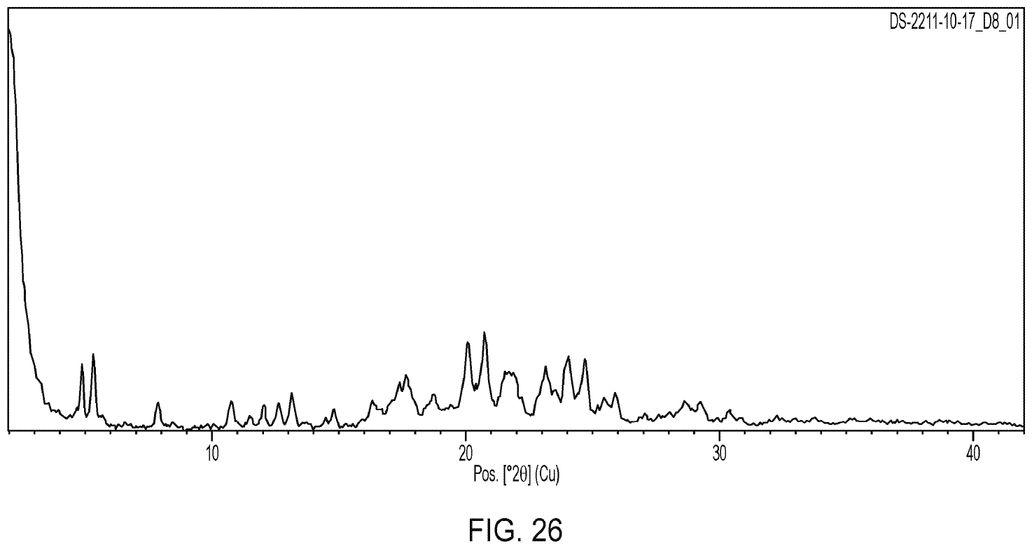

In certain embodiments, Form A of compound I-11 has an X-Ray diffraction pattern substantially similar to that depicted in FIG. 26. In some embodiments, Form A of Compound I-11 may be characterized by a powder X-ray diffraction pattern with at least two characteristic peaks, in degrees 2θ, each selected from the group consisting of the peaks listed in Table 14. In some embodiments, Form A of Compound I-11 may be characterized by a powder X-ray diffraction pattern with at least three characteristic peaks, in degrees 2θ, each selected from the group consisting of the peaks listed in Table 14. In some embodiments, Form A of Compound I-11 may be characterized by a powder X-ray diffraction pattern with at least four characteristic peaks, in degrees 2θ, each selected from the group consisting of the peaks listed in Table 14. In some embodiments, Form A of Compound I-11 may be characterized by a powder X-ray diffraction pattern with at least five characteristic peaks, in degrees 2θ, each selected from the group consisting of the peaks listed in Table 14. In some embodiments, Form A of Compound I-11 may be characterized by a powder X-ray diffraction pattern with at least six characteristic peaks, in degrees 2θ, each selected from the group consisting of the peaks listed in Table 14.

In certain embodiments, a solid crystalline form of compound I-11 has an X-Ray diffraction pattern substantially similar to any one of the patterns depicted in FIG. 25.

In some embodiments, the solid crystalline form of Compound I-11 is Form B. In some embodiments, Form B of Compound I-11 may be characterized by a powder X-ray diffraction pattern with at least two characteristic peaks, in degrees 2θ, each selected from the group consisting of about 20.2 2θ, about 24.1 2θ and about 4.9 2θ. In some embodiments, Form B of Compound I-11 may be characterized by a powder X-ray diffraction pattern with at least two characteristic peaks, in degrees 2θ, each selected from the group consisting of about 20.2 2θ, about 24.1 2θ, about 4.9 2θ, about 23.2 2θ, about 12.7 2θ, about 17.0 2θ and about 16.3 2θ. In some embodiments, Form B of Compound I-11 may be characterized by a powder X-ray diffraction pattern with at least three characteristic peaks, in degrees 2θ, each selected from the group consisting of about 20.2 2θ, about 24.1 2θ, about 4.9 2θ, about 23.2 2θ, about 12.7 2θ, about 17.0 2θ and about 16.3 2θ. In some embodiments, Form B of Compound I-11 may be characterized by a powder X-ray diffraction pattern with at least four characteristic peaks, in degrees 2θ, each selected from the group consisting of about 20.2 2θ, about 24.1 2θ, about 4.9 2θ, about 23.2 2θ, about 12.7 2θ, about 17.0 2θ and about 16.3 2θ. In some embodiments, Form B of Compound I-11 may be characterized by a powder X-ray diffraction pattern with at least five characteristic peaks, in degrees 2θ, each selected from the group consisting of about 20.2 2θ, about 24.1 2θ, about 4.9 2θ, about 23.2 2θ, about 12.7 2θ, about 17.0 2θ and about 16.3 2θ. In some embodiments, Form B of Compound I-11 may be characterized by a powder X-ray diffraction pattern with at least six characteristic peaks, in degrees 2θ, each selected from the group consisting of about 20.2 2θ, about 24.1 2θ, about 4.9 2θ, about 23.2 2θ, about 12.7 2θ, about 17.0 2θ and about 16.3 2θ.

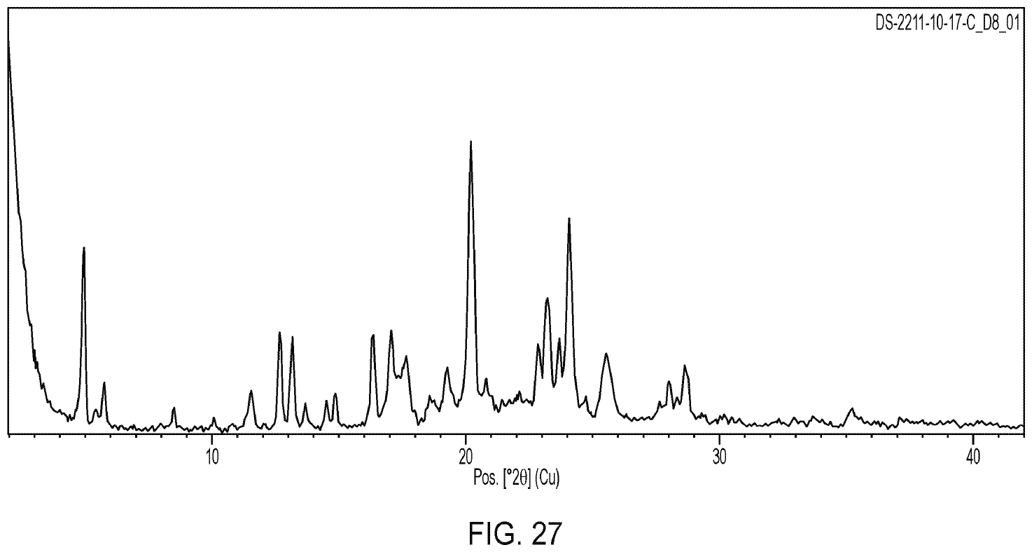

In certain embodiments, Form B of compound I-11 has an X-Ray diffraction pattern substantially similar to that depicted in FIG. 27. In some embodiments, Form B of Compound I-11 may be characterized by a powder X-ray diffraction pattern with at least two characteristic peaks, in degrees 2θ, each selected from the group consisting of the peaks listed in Table 15. In some embodiments, Form B of Compound I-11 may be characterized by a powder X-ray diffraction pattern with at least three characteristic peaks, in degrees 2θ, each selected from the group consisting of the peaks listed in Table 15. In some embodiments, Form B of Compound I-11 may be characterized by a powder X-ray diffraction pattern with at least four characteristic peaks, in degrees 2θ, each selected from the group consisting of the peaks listed in Table 15. In some embodiments, Form B of Compound I-11 may be characterized by a powder X-ray diffraction pattern with at least five characteristic peaks, in degrees 2θ, each selected from the group consisting of the peaks listed in Table 15. In some embodiments, Form B of Compound I-11 may be characterized by a powder X-ray diffraction pattern with at least six characteristic peaks, in degrees 2θ, each selected from the group consisting of the peaks listed in Table 15.

In certain embodiments, a solid crystalline form of compound I-11 has an X-Ray diffraction pattern substantially similar to any one of the patterns depicted in FIG. 27.

### Methods

Compounds and compositions described herein are generally useful for the inhibition of a kinase or a mutant thereof. In some embodiments, the kinase inhibited by the compounds and compositions described herein is a cyclin dependent kinase (CDK). In some embodiments, the kinase inhibited by the compounds and compositions described herein is one or more of CDK1, CDK2, CDK4, and CDK6. In some embodiments, the kinase inhibited by the compounds and compositions described herein is CDK2.

Compounds or compositions of the disclosure can be useful in applications that benefit from inhibition of CDK2 enzymes. For example, CDK2 inhibitors described herein are useful for the treatment of proliferative diseases generally.

CDK2 is known to be an factor in tumorigenesis and proliferation in many cancer types including lung cancer, liver cancer, colon cancer and breast cancer (Opyrchal, Int J Oncol 2014; Shi, PLoS One 2015; Lim, Cancer Prev Res 2014). There is evidence showing that CDK2 is functionally linked with hyper proliferation in multiple cancer cells and is a potential therapeutic target for cancer therapy (Chohan, Curr Med Chem 2015).

CDK2 plays a role for the malignant transformation of breast epithelial cells. Suppression of CDK2 activity can effectively inhibit the proliferation of human breast cancer cells (Ali, Cancer Res 2009). Active CDK2 in the form of a cyclin D1/CDK2 fusion protein induces tumors that contain an invasive component that exhibits multiple features in common with human basal-like tumors and tumor-derived cell lines (Corsino, Neoplasia 2008). Cyclin D1/CDK2 complexes were detected in human breast cancer cell lines (Sweeney, Oncogene 1998), and the levels of these complexes correlated well with the degree of cyclin D1 overexpression.

The role of cyclin E and its associated kinase CDK2 in ovarian cancer has been investigated by screening primary, metastatic, recurrent and benign ovarian tumors. Using gene amplification, Cyclin E was shown to be amplified in 21% and CDK2 in 6.4% of the cases analyzed. Additionally, Cyclin E RNA was overexpressed in 29.5% and CDK2 in 6.5% of ovarian tumors tested. Cyclin E and CDK2 were overexpressed mostly in primary ovarian cancers (32% and 10%, respectively) compared to metastatic and recurrent diseases (Marone, Int J Cancer 1998).

CDK2 expression has been found to be significantly elevated in glioma tumor especially in Glioblastoma Multiforme (GBM) and was functionally required for GBM cell proliferation and tumorigenesis (Wang, Transl Oncol 2016). CDK2 expression was identified to be significantly enriched in GBM tumors and functionally required for tumor proliferation both in vitro and in vivo. Additionally, high CDK2 expression was associated to poor prognosis in GBM patients. Radio resistance is a major factor of poor clinical prognosis and tumor recurrence in GBM patients. CDK2 was found to be one of the most up-regulated kinase encoding genes in GBM after radio treatment. CDK2-dependent radio resistance is indispensable for GBM tumorigenesis and recurrence after therapeutic treatment (Id).

Elevated levels of CDK2 expression have been observed in human cholangiocarcinoma tissues where apoptosis-related protein-1 dependent suppression of CDK2 induced cell cycle arrest and restrained tumor growth (Zheng, Oncol Rep 2016).

CDK2 overexpression in oral squamous cell carcinoma (SCC) may elevate pRB phosphorylation and permit more rapid entry of the cancer cells into S phase. In a clinicopathological survey of oral SCC, incidence of CDK2 expression was high in the poorly differentiated lesions, and was associated with the mode of tumor invasion, lymph node involvement and survival, an indication that change in CDK2 expression is associated with oral cancer progression (Mihara, Jpn J Cancer Res 2001). CDK2 expression was significantly correlated with lymph node involvement, tumor differentiation, mode of tumor invasion, and shorter survival period. Thus, increased expression of CDK2 is a factor in oral cancer progression and a negative predictive marker of the patients' prognosis (Id).

CDK2 has been found to play a role in cell proliferation of non-small cell lung cancer (Kawana, Am J Pathol 1998). CDK2 has also been found to play a role in cell proliferation of prostate cancer (Flores, Endocrinology 2010).

The activity of a compound described herein as an inhibitor of an CDK kinase, for example, CDK2, or a mutant thereof, may be assayed in vitro, in vivo or in a cell line. In vitro assays include assays that determine inhibition of either the phosphorylation activity and/or the subsequent functional consequences, or ATPase activity of activated CDK2, or a mutant thereof. Alternative in vitro assays quantitate the ability of the inhibitor to bind to CDK2. Inhibitor binding may be measured by radiolabeling the inhibitor prior to binding, isolating the inhibitor/CDK2 complex and determining the amount of radiolabel bound. Alternatively, inhibitor binding may be determined by running a competition experiment where new inhibitors are incubated with CDK2 bound to known radioligands. Representative in vitro and in vivo assays useful in assaying an CDK2 inhibitor include those described and disclosed in the patent and scientific publications described herein. Detailed conditions for assaying a compound described herein as an inhibitor of CDK2, or a mutant thereof, are set forth in the Examples below.

### Treatment of Disorders

Provided compounds are inhibitors of CDK2 and are therefore useful for treating one or more disorders associated with activity of CDK2 or mutants thereof. Thus, in certain embodiments, the present disclosure provides a method of treating an CDK2-mediated disorder in a subject comprising administering a therapeutically effective amount of a compound described herein, or a pharmaceutically acceptable salt thereof, or a pharmaceutically acceptable composition of either of the foregoing, to a subject in need thereof. In certain embodiments, the present disclosure provides a method of treating an CDK2-mediated disorder in a subject comprising administering a therapeutically effective amount of a compound described herein, or a pharmaceutically acceptable composition thereof, to a subject in need thereof.

As used herein, the term “CDK2-mediated” disorders, diseases, and/or conditions means any disease or other deleterious condition in which CDK2 or a mutant thereof is known to play a role. Accordingly, another embodiment of the present disclosure relates to treating or lessening the severity of one or more diseases in which CDK2, or a mutant thereof, is known to play a role. Such CDK2-mediated disorders include but are not limited to proliferative disorders (e.g. cancer).

In some embodiments, the present disclosure provides a method for treating one or more disorders, wherein the disorders are selected from proliferative disorders and craniosynostotic syndromes, said method comprising administering to a patient in need thereof, a therapeutically effective amount of a compound of the present disclosure, or a pharmaceutically acceptable salt thereof, or a pharmaceutically acceptable composition of either of the foregoing. In some embodiments, the present disclosure provides a method for treating one or more disorders, wherein the disorders are selected from proliferative disorders and craniosynostotic syndromes, said method comprising administering to a patient in need thereof, a therapeutically effective amount of a compound of the present disclosure, or a pharmaceutically acceptable composition thereof.

In some embodiments, the disorder is associated with CDK2 signaling. CDK2 is known to have multiple upstream and downstream signaling pathways and inhibition of CDK2 can be used to treat disorders associated with aberrant signaling within those pathways. In some embodiments, the disorder is associated with cyclin E, cyclin E1, or retinoblastoma protein (RB) signaling.

In some embodiments, the method of treatment comprises the steps of: (i) identifying a subject in need of such treatment; (ii) providing a disclosed compound, or a pharmaceutically acceptable salt thereof, and (iii) administering said provided compound in a therapeutically effective amount to treat, suppress and/or prevent the disease state or condition in a subject in need of such treatment.

In some embodiments, the method of treatment comprises the steps of: (i) identifying a subject in need of such treatment; (ii) providing a composition comprising a disclosed compound, or a pharmaceutically acceptable salt thereof; and (iii) administering said composition in a therapeutically effective amount to treat, suppress and/or prevent the disease state or condition in a subject in need of such treatment.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

### Proliferative Disorders

In some embodiments, the disorder is a proliferative disorder. In some embodiments, the proliferative disorder is cancer. In some embodiments, the proliferative disorder is ovarian cancer, breast cancer, lung cancer, colorectal cancer, or a combination thereof. In some embodiments, the proliferative disorder is a leukemia. In some embodiments, the proliferative disorder is breast cancer. In some embodiments, the proliferative disorder is a lung cancer. In some embodiments, the proliferative disorder is colorectal cancer.

In some embodiments, the proliferative disorder is breast cancer, prostate cancer, lung squamous cell carcinoma, thyroid cancer, gastric cancer, ovarian cancer, rectal cancer, endometrial carcinoma, non-small cell lung cancer, or bladder cancer. In some embodiments, the proliferative disorder is intrahepatic cholangiocarcinoma, hepatocellular carcinoma, breast cancer, prostate cancer, lung squamous cell carcinoma, thyroid cancer, gastric cancer, or ovarian cancer. In some embodiments, the proliferative disorder is gastric cancer, breast cancer, triple negative breast cancer, or rectal cancer. In some embodiments, the proliferative disorder is endometrial carcinoma, non-small cell lung cancer, lung squamous cell carcinoma, gastric cancer, breast cancer, or urothelial cancer.

In some embodiments, the disorder is ovarian cancer, endometrial cancer, gastric cancer, breast cancer, lung cancer, bladder cancer, cervical cancer, stomach cancer, sarcoma cancer, liver cancer, esophageal cancer, laryngeal cancer, multiple myeloma, colorectal cancer, rectal cancer, skin cancer, or pancreatic cancer. In some embodiments, the bladder cancer is urothelial carcinoma. In some embodiments, the liver cancer is hepatocellular carcinoma. In some embodiments, the lung cancer is lung squamous cell carcinoma or non-small cell lung cancer. In some embodiments, the laryngeal cancer is laryngeal squamous cell carcinoma. In some embodiments, the skin cancer is melanoma.

In some embodiments, the proliferative disorder is associated with a deregulation of CDK2 or cyclin E. In some embodiments, the deregulation of CDK2 is an overexpression of CDK2 or cyclin E. In some embodiments, the deregulation of cyclin E is an overexpression of CDK2 or cyclin E. In some embodiments, the proliferative disorder is associated with a deregulation of CDK2 and cyclin E. In some embodiments, the deregulation of CDK2 and cyclin E is an overexpression of CDK2 and cyclin E.

In some embodiments, the proliferative disorder is associated with one or more activating mutations in CDK2. In some embodiments, the activating mutation in CDK2 is a mutation to one or more of the intracellular kinase domain and the extracellular domain. In some embodiments, the activating mutation in CDK2 is a mutation to the intracellular kinase domain.

### Routes of Administration and Dosage Forms

The compounds and compositions, according to the methods described herein, may be administered using any amount and any route of administration effective for treating or lessening the severity of the disorder (e.g. a proliferative disorder or craniosynostotic syndrome). The exact amount required will vary from subject to subject, depending on the species, age, and general condition of the subject, the severity of the infection, the particular agent, its mode of administration, and the like. Compounds described herein are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions described herein can be administered to humans and other animals orally, rectally, parenterally, intracisternally, intravaginally, intraperitoneally, topically (as by powders, ointments, or drops), bucally, as an oral or nasal spray, or the like. In certain embodiments, the compounds described herein may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Dosage Amounts and Regimens

In accordance with the methods of the present disclosure, the compounds of the disclosure are administered to the subject in a therapeutically effective amount, e.g., to reduce or ameliorate symptoms of the disorder in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, in some embodiments, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

### Inhibition of Protein Kinases

According to one embodiment, the present disclosure relates to a method of inhibiting protein kinase activity in a biological sample comprising the step of contacting said biological sample with a compound described herein, or a composition comprising said compound.

According to another embodiment, the present disclosure relates to a method of inhibiting activity of CDK2, or a mutant thereof, in a biological sample comprising the step of contacting said biological sample with a compound described herein, or a composition comprising said compound. In certain embodiments, the present disclosure relates to a method of reversibly inhibiting CDK2, or a mutant thereof, activity in a biological sample comprising the step of contacting said biological sample with a compound described herein, or a composition comprising said compound.

In another embodiment, the present disclosure provides a method of selectively inhibiting CDK2 over one or more of CDK1, CDK4, CDK5, CDK6, and CDK9. In some embodiments, a compound described herein is more than 5-fold selective over CDK1, CDK4, CDK5, CDK6, and CDK9. In some embodiments, a compound described herein is more than 10-fold selective over CDK1, CDK4, CDK5, CDK6, and CDK9. In some embodiments, a compound described herein is more than 50-fold selective over CDK1, CDK4, CDK5, CDK6, sand CDK9. In some embodiments, a compound described herein is more than 100-fold selective over CDK1, CDK4, CDK5, CDK6, and CDK9. In some embodiments, a compound described herein is more than 200-fold selective over CDK1, CDK4, CDK5, CDK6, and CDK9.

The term “biological sample”, as used herein, includes, without limitation, cell cultures or extracts thereof, biopsied material obtained from a mammal or extracts thereof, and blood, saliva, urine, feces, semen, tears, or other body fluids or extracts thereof.

Inhibition of activity of CDK2 (or a mutant thereof) in a biological sample is useful for a variety of purposes that are known to one of skill in the art. Examples of such purposes include, but are not limited to, blood transfusion, organ-transplantation, biological specimen storage, and biological assays.

Another embodiment of the present disclosure relates to a method of inhibiting protein kinase activity in a patient comprising the step of administering to said patient a compound described herein, or a composition comprising said compound.

According to another embodiment, the present disclosure relates to a method of inhibiting activity of CDK2, or a mutant thereof, in a patient comprising the step of administering to said patient a compound described herein, or a composition comprising said compound. According to certain embodiments, the present disclosure relates to a method of reversibly inhibiting activity of one or more of CDK2, or a mutant thereof, in a patient comprising the step of administering to said patient a compound described herein, or a composition comprising said compound.

According to another embodiment, the present disclosure provides a method for treating a disorder mediated by CDK2, or a mutant thereof, in a patient in need thereof, comprising the step of administering to said patient a compound described herein or a pharmaceutically acceptable composition thereof. Such disorders are described in detail herein. In some embodiments, the present disclosure provides a method for treating a disorder mediated by CDK2, or a mutant thereof, in a patient in need thereof, comprising the step of administering to said patient a compound described herein or a pharmaceutically acceptable composition thereof, wherein the compound reversibly inhibits the CDK2, or a mutant thereof.

According to another embodiment, the present disclosure provides a method of inhibiting signaling activity of CDK2, or a mutant thereof, in a subject, comprising administering a therapeutically effective amount of a compound described herein, or a pharmaceutically acceptable composition thereof, to a subject in need thereof. In some embodiments, the present disclosure provides a method of inhibiting CDK2 signaling activity in a subject, comprising administering a therapeutically effective amount of a compound described herein, or a pharmaceutically acceptable composition thereof, to a subject in need thereof.

In some embodiments, the present disclosure provides a method for treating a disorder mediated by CDK2, or a mutant thereof, in a patient in need thereof, comprising the step of administering to said patient a compound described herein or a pharmaceutically acceptable composition thereof, wherein the compound reversibly inhibits the CDK2, or a mutant thereof.

In some embodiments, a patient is 18 years or older. In some embodiments, a patient has a disease that is refractory to standard therapy. In some embodiments, a patient has a disease that has not adequately responded to standard therapy. In some embodiments, a patient has a disease for which standard or curative therapy does not exist. In some embodiments, a patient is intolerant to or have declined standard therapy.

The compounds described herein can also inhibit CDK2 function through incorporation into agents that catalyze the destruction of CDK2. For example, the compounds can be incorporated into proteolysis targeting chimeras (PROTACs). A PROTAC is a bifunctional molecule, with one portion capable of engaging an E3 ubiquitin ligase, and the other portion having the ability to bind to a target protein meant for degradation by the cellular protein quality control machinery. Recruitment of the target protein to the specific E3 ligase results in its tagging for destruction (i.e., ubiquitination) and subsequent degradation by the proteasome. Any E3 ligase can be used. The portion of the PROTAC that engages the E3 ligase is connected to the portion of the PROTAC that engages the target protein via a linker which consists of a variable chain of atoms. Recruitment of CDK2 to the E3 ligase will thus result in the destruction of the CDK2 protein. The variable chain of atoms can include, for example, rings, heteroatoms, and/or repeating polymeric units. It can be rigid or flexible. It can be attached to the two portions described above using standard techniques in the art of organic synthesis.

### Combination Therapies

Depending upon the particular disorder, condition, or disease, to be treated, additional therapeutic agents, that are normally administered to treat that condition, may be administered in combination with compounds and compositions described herein. As used herein, additional therapeutic agents that are normally administered to treat a particular disease, or condition, are known as “appropriate for the disease, or condition, being treated.”

Accordingly, in certain embodiments, the method of treatment comprises administering the compound or composition described herein in combination with one or more additional therapeutic agents. In certain other embodiments, the methods of treatment comprise administering the compound or composition described herein as the only therapeutic agent.

In some embodiments, compounds or compositions of the disclosure can be used in combination with other treatments and/or cancer therapies. For example, compounds or compositions of the disclosure can be used in combination with, but are not limited to, antibodies, antibody-drug conjugates, kinase inhibitors, immunomodulators, and histone deacetylase inhibitors. The compounds or compositions of the disclosure can also be used in combination with other treatments and/or cancer therapies as disclosed in WO 2018/057884, WO 2015/107495, WO 2018/172984, WO 2018/136265, and WO 2021/222556; and references cited therein; each of which is hereby incorporated by reference in its entirety. For example, SHP099, RLY1971, RMC-4550, RMC4630, JAB3068, JAB3312, or TNO155.

In some embodiments, compounds or compositions of the disclosure can be used in combination with a compound of the formula:

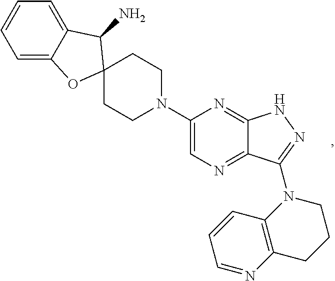 [SMILES: N[C@@H]1c2ccccc2OC12CCN(c1cnc3c(N4CCCc5ncccc54)n[nH]c3n1)CC2]

or pharmaceutically acceptable salts thereof.

For example, the compounds disclosed herein (or pharmaceutical compositions containing them) can be used in the treatment of one or more of the diseases mentioned herein, alone or in combination with another therapeutic agent. For example, a compound of Formula (I) can be used in combination with the following agents: BCR-ABL inhibitors: imatinib mesylate; inilotinib hydrochloride; nilotinib; dasatinib; bosutinib; ponatinib; bafetinib; danusertib; saracatinib; N-[2-[(1S,4R)-6-[[4-(Cyclobutylamino)-5-(trifluoromethyl)-2-pyrimidinyl]amino]-1,2,3,4-tetrahydronaphthalen-1,4-imin-9-yl]-2-oxoethyl]-acetamide. ALK inhibitors: crizotinib; 5-chloro-N4-(2-(isopropylsulfonyl)phenyl)-N2-(2-methoxy-4-(4-(4-methylpiperazin-1-yl)piperidin-1-yl)phenyl)pyrimidine-2,4-diamine, ceritinib, alectinib, brigatinib, entrecinib. BRAF inhibitors: vemurafenib and dabrafenib. FGFR inhibitors: infigratinib, dovitinib, erdafitinib, BLU-554, AZD4547. FLT3 inhibitors: sunitinib malate; midostaurin; tanutinib; sorafenib, lestaurtinib, quizartinib and crenolanib. KRAS inhibitors: MRTX849, AMG510. MEK Inhibitors—trametinib, combimetinib, binimetinib, selumetinib. VEGF receptor inhibitors: bevacizumab, axitinib, Aflibercept, (N-methyl-2-[[3-[(E)-2-pyridin-2-ylethenyl]-1H-indazol-6-yl]sulfanyl]benzamide, brivanib alaninate ((S)-((R)-1-(4-(4-Fluoro-2-methyl-1H-indol-5-yloxy)-5-methylpyrrolo[2,1-f][1,2,4]triazin-6-yloxy)propan-2-yl)2-aminopropanoate, motesanib (N-(2,3-dihydro-3,3-dimethyl-1H-indol-6-yl)-2-[(4-pyridinylmethyl)amino]-3-pyridinecarboxamide, pasireotide, sorafenib. Tyrosine kinase inhibitors: erlotinib hydrochloride, linifanib, sunitinib malate, pazopanib. Epidermal growth factor receptor (EGFR) inhibitors: Gefitnib, osimertinib, cetuximab, panitumumab. HER2 receptor inhibitors: trastuzumab, neratinib, lapatinib or lapatinib ditosylate. MET inhibitors: crizotinib, cabozantinib. CD20 antibodies: rituximab, tositumomab, ofatumumab. DNA Synthesis inhibitors: capecitabine, gemcitabine hydrochloride, nelarabine, hydroxycarbamide. Antineoplastic agents: oxaliplatin. HER dimerization inhibitors: pertuzumab. Human Granulocyte colony-stimulating factor (G-CSF) modulators: Filgrastim. Immunomodulators: Afutuzumab, lenalidomide, thalidomide. CD40 inhibitors: Dacetuzumab. Pro-apoptotic receptor agonists (PARAs): Dulanermin. Heat Shock Protein (HSP) inhibitors: Tanespimycin (17-allylamino-17-demethoxygeldanamycin). Hedgehog antagonists: 2-chloro-N-[4-chloro-3-(2-pyridinyl)phenyl]-4-(methylsulfonyl)-benzamide. Proteasome inhibitors: Bortezomib. PI3K inhibitors: 4-[2-(1H-Indazol-4-yl)-6-[[4-(methylsulfonyl)piperazin-1-yl]methyl]thieno[3,2-d]pyrimidin-4-yl]mo choline, 2-Methyl-2-[4-[3-methyl-2-oxo-8-(quinolin-3-yl)-2,3-dihydroimidazo[4,5-c]quinolin-1-yl]phenyl]propiocyano, buparlisib, taselisib, idelalisib, duvelisib, TGR 1202. Phospholipase A2 inhibitors: Anagrelide. BCL-2 inhibitors: 4-[4-[[2-(4-chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3-[(trifluoromethyl)sulfonyl]phenyl]sulfonyl]benzamide. Mitogen-activated protein kinase kinase (MEK) inhibitors: XL-518. Aromatase inhibitors: Exemestane, letrozole, anastrozole, faslodex, tamoxifen. Topoisomerase I inhibitors: Irinotecan, topotecan hydrochloride. Topoisomerase II inhibitors: etoposide, teniposide. mTOR inhibitors: Temsirolimus, ridaforolimus, everolimus. Osteoclastic bone resorption inhibitors: 1-Hydroxy-2-imidazol-1-yl-phosphonoethyl) phosphonic acid monohydrate. CD33 Antibody Drug Conjugates: Gemtuzumab ozogamicin. CD22 Antibody Drug Conjugates: Inotuzumab ozogamicin. CD20 Antibody Drug Conjugates: Ibritumomab tiuxetan. Somatostain analogs: octreotide. Synthetic Interleukin-11 (IL-11): oprelvekin. Synthetic erythropoietin: Darbepoetin alfa. Receptor Activator for Nuclear Factor κ B (RANK) inhibitors: Denosumab. Thrombopoietin mimetic peptides: Romiplostim. Cell growth stimulators: Palifermin. Anti-Insulin-like Growth Factor-1 receptor (IGF-1R) antibodies: Figitumumab. Anti-CSI antibodies: Elotuzumab. CD52 antibodies: Alemtuzumab. CTLA-4 inhibitors: Tremelimumab, ipilimumab. PD1 inhibitors: Nivolumab; pembrolizumab; an immunoadhesin; Pidilizumab; and AMP-224. PDL1 inhibitors: MSB0010718C; YW243.55.S70, MPDL3280A; MEDI-4736, MSB-0010718C, or MDX-1105. LAG-3 inhibitors: BMS-986016. GITR agonists: GITR fusion proteins and anti-GITR antibodies. Histone deacetylase inhibitors (HDI): Voninostat. Anti-CTLA4 antibodies: Tremelimumab; and Ipilimumab. Alkylating agents: Temozolomide, dactinomycin, melphalan, altretamine carmustine, bendamustine, busulfan, carboplatin, lomustine, cisplatin, chlorambucil, cyclophosphamide, dacarbazine, altretamine, ifosfamide, procarbazine, mechlorethamine, mustine and mechloroethamine hydrochloride, streptozocin, thiotepa. Biologic response modifiers: bacillus calmette-guerin, denileukin diftitox. Anti-tumor antibiotics: doxorubicin, bleomycin, daunorubicin, daunorubicin liposomal, mitoxantrone, epirubicin, idarubicin, mitomycin C. Anti-microtubule agents: Estramustine. Cathepsin K inhibitors: Odanacatib. Epothilone B analogs: Ixabepilone. TpoR agonists: Eltrombopag. Anti-mitotic agents: Docetaxel. Adrenal steroid inhibitors: aminoglutethimide. Anti-androgens: Nilutamide, Androgen Receptor inhibitors: enzalutamide, abiraterone acetate, orteronel, galeterone, and seviteronel, bicalutamide, flutamide. Androgens: Fluoxymesterone. CDK inhibitors: Alvocidib, palbociclib, ribociclib, trilaciclib, abemaciclib. TRK inhibitors: entrectinib, larotrectinib. RET inhibitors: BLU-667, LOXO-292. Gonadotropin-releasing hormone (GnRH) receptor agonists: Leuprolide or leuprolide acetate. Taxane anti-neoplastic agents: Cabazitaxel (1-hydroxy, 10-dimethoxy-9-oxo-5,20-epoxytax-1 1-ene-2a,4,13a-triyl-4-acetate-2-benzoate-13-[(2R,3S)-3-{[(tert-butoxy)carbonyl]amino}-2-hydroxy-3-phenylpropanoate), larotaxel ((2α,5β,7β,10β,13α)-4,10-diacetoxy-1-hydroxy-13-{[(2R,3S)-2-hydroxy-3-({[(2-methyl-2-propanyl)oxy]carbonyl}amino)-3-phenylpropanoyl]oxy}-9-oxo-5,20-epoxy-7,19-cyclotax-11-en-2-yl benzoate). 5HTla receptor agonists: Xaliproden (also known as SR57746, 1-[2-(2-naphthyl)ethyl]-4-[3-(trifluoromethyl)phenyl]-1,2,3,6-tetrahydropyridine.
HPC vaccines: Cervarix® sold by GlaxoSmithKline, Gardasil® sold by Merck;
Iron Chelating agents: Deferasinox. Anti-metabolites: Claribine (2-chlorodeoxyadenosine), 5-fluorouracil, 6-thioguanine, pemetrexed, cytarabine, cytarabine liposomal, decitabine, hydroxyurea, fludarabine, floxuridine, cladribine, methotrexate, pentostatin. Bisphosphonates: Pamidronate. Demethylating agents: 5-azacitidine, decitabine.
Plant Alkaloids: Paclitaxel protein-bound; vinblastine, vincristine, vinorelbine, paclitaxel.
Retinoids: Alitretinoin (sold under the tradename Panretin®), tretinoin (all-trans retinoic acid, also known as ATRA, sold under the tradename Vesanoid®), Isotretinoin (13-cis-retinoic acid, sold under the tradenames Accutane®, Amnesteem®, Claravis®, Claras®, Decutan®, Isotane®, Izotech®, Oratane®, Isotret®, and Sotret®), bexarotene (sold under the tradename Targretin®). Glucocorticosteroids: Hydrocortisone (also known as cortisone, hydrocortisone sodium succinate, hydrocortisone sodium phosphate, and sold under the tradenames Ala-Cort®, Hydrocortisone Phosphate, Solu-Cortef®, Hydrocort Acetate® and Lanacort®), dexamethazone ((8S,9R,10S,11S,13S,14S,16R,17R)-9-fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-Methylprednisolone, Methylprednisolone Acetate, Methylprednisolone Sodium Succinate, sold under the tradenames Duralone®, Medralone®, Medrol®, M-Prednisol® and Solu-Medrol®). Cytokines: interleukin-2 (also known as aldesleukin and IL-2, sold under the tradename Proleukin®), interleukin-11 (also known as oprevelkin, sold under the tradename Neumega®), alpha interferon alfa (also known as IFN-alpha, sold under the tradenames Intron® A, and Roferon-A®). Estrogen receptor downregulators: Fulvestrant (sold under the tradename Faslodex®). Anti-estrogens: tamoxifen (sold under the tradename Novaldex®). Toremifene (sold under the tradename Fareston®).
Selective estrogen receptor modulators (SERMs): Raloxifene (sold under the tradename Evista®). Leutinizing hormone releasing hormone (LHRH) agonists: Goserelin (sold under the tradename Zoladex®);
Progesterones: megestrol (also known as megestrol acetate, sold under the tradename Megace®); Miscellaneous cytotoxic agents: Arsenic trioxide (sold under the tradename Trisenox®), asparaginase (also known as L-asparaginase, Erwinia L-asparaginase, sold under the tradenames Elspar® and Kidrolase®). Anti-nausea drugs: NK-1 receptor antagonists: Casopitant (sold under the tradenames Rezonic® and Zunrisa® by GlaxoSmithKline); and
Cytoprotective agents: Amifostine (sold under the tradename Ethyol®), leucovorin (also known as calcium leucovorin, citrovorum factor and folinic acid). Immune checkpoint inhibitors: The term “immune checkpoints” refers to a group of molecules on the cell surface of CD4 and CD8 T cells. Immune checkpoint molecules include, but are not limited to, Programmed Death 1 (PD-1), Cytotoxic T-Lymphocyte Antigen 4 (CTLA-4), B7H1, B7H4, OX-40, CD 137, CD40, and LAG3. Immunotherapeutic agents which can act as immune checkpoint inhibitors useful in the methods of the present disclosure, include, but are not limited to, inhibitors of PD-L1, PD-L2, CTLA4, TIM3, LAG3, VISTA, BTLA, TIGIT, LAIR1, CD 160, 2B4 and/or TGFR beta.

The compounds described herein can be linked to one end of a variable chain, while the other end of the variable chain can be bound to the E3 ligase. Recruitment of CDK2 to the ligase will thus result in the destruction of the CDK2 protein.

In some embodiments, compounds or compositions of the disclosure can be used in combination with an antibody. In some embodiments, compounds or compositions of the disclosure can be used in combination with an antibody-drug conjugate. In some embodiments, compounds or compositions of the disclosure can be used in combination with a kinase inhibitor. In some embodiments, compounds or compositions of the disclosure can be used in combination with an immunomodulator. In some embodiments, compounds or compositions of the disclosure can be used in combination with a histone deacetylase inhibitor.

In some embodiments, disclosed compounds can be administered to a subject in need of treatment at dosages ranging from about 0.0001 mg to about 100 mg/kg body weight of the subject to be treated per day, such as from about 1.0 to 10 mg/kg. However, additional variations are within the scope of the disclosure.

A disclosed compound can be administered alone or in combination with pharmaceutically acceptable carriers, such as diluents, fillers, aqueous solution, and even organic solvents. The compound and/or compositions of the disclosure can be administered as a tablet, powder, lozenge, syrup, injectable solution, and the like. Additional ingredients, such as flavoring, binder, excipients, and the like are within the scope of the disclosure.

In some embodiments, the present disclosure provides for the use of pharmaceutical compositions and/or medicaments comprised of a compound disclosed herein, or a pharmaceutically acceptable salt thereof, in a method of treating a disease state, and/or condition caused by or related to CDK2 kinase. For example, provided herein are methods of treating subjects in need thereof (e.g., subjects suffering from cancer (e.g., leukemia, breast, lung and/or colorectal cancer) an effective amount of a disclosed compound, and optionally an effective amount of an additional compound (e.g., therapeutic agent) such as disclosed herein.

In some embodiments, a method of treatment comprises the steps of: (i) identifying a subject in need of such treatment; (ii) providing a compound disclosed herein, or a pharmaceutically acceptable salt thereof, and (iii) administering said compound in a therapeutically effective amount to treat, suppress and/or prevent the disease state or condition in a subject in need of such treatment.

In some embodiments, a method of treatment comprises the steps of: (i) identifying a subject in need of such treatment; (ii) providing a composition comprising a compound disclosed herein, or a pharmaceutically acceptable salt thereof, and (iii) administering said composition in a therapeutically effective amount to treat, suppress and/or prevent the disease state or condition in a subject in need of such treatment.

In some embodiments, the subject is an animal. Animals include all members of the animal kingdom, but are not limited to humans, mice, rats, cats, monkeys, dogs, horses, and swine. In some embodiments, the subject is a human. In some embodiments, the subject is a mouse, a rat, a cat, a monkey, a dog, a horse, or a pig.

In some embodiments, the method of treatment, prevention and/or suppression of a condition related to CDK2 comprises the steps of: (i) identifying a subject in need of such treatment; (ii) providing a compound disclosed herein or a pharmaceutically acceptable salt thereof, or a composition comprising a compound disclosed herein, or a pharmaceutically acceptable salt thereof, and a pharmaceutically acceptable carrier; and (iii) administering said compound or composition in a therapeutically effective amount to treat, prevent and/or suppress the disease state or condition related to CDK2 in a subject in need of such treatment.

In accordance with the methods of the present disclosure, the compounds of the disclosure are administered to the subject in a therapeutically effective amount, e.g., to reduce or ameliorate symptoms related to CDK2 kinase activity in the subject. This amount is readily determined by the skilled artisan, based upon known procedures, including analysis of titration curves established in vivo and methods and assays disclosed herein.

In some embodiments, the methods comprise administration of a therapeutically effective dosage of the compounds of the disclosure. In some embodiments, the therapeutically effective dosage is at least about 0.0001 mg/kg body weight, at least about 0.001 mg/kg body weight, at least about 0.01 mg/kg body weight, at least about 0.05 mg/kg body weight, at least about 0.1 mg/kg body weight, at least about 0.25 mg/kg body weight, at least about 0.3 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 350 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. It will be recognized that any of the dosages listed herein may constitute an upper or lower dosage range, and may be combined with any other dosage to constitute a dosage range comprising an upper and lower limit.

In some embodiments, the therapeutically effective dosage is in the range of about 0.1 mg to about 10 mg/kg body weight, about 0.1 mg to about 6 mg/kg body weight, about 0.1 mg to about 4 mg/kg body weight, or about 0.1 mg to about 2 mg/kg body weight.

In some embodiments the therapeutically effective dosage is in the range of about 1 to 500 mg, about 2 to 150 mg, about 2 to 120 mg, about 2 to 80 mg, about 2 to 40 mg, about 5 to 150 mg, about 5 to 120 mg, about 5 to 80 mg, about 10 to 150 mg, about 10 to 120 mg, about 10 to 80 mg, about 10 to 40 mg, about 20 to 150 mg, about 20 to 120 mg, about 20 to 80 mg, about 20 to 40 mg, about 40 to 150 mg, about 40 to 120 mg or about 40 to 80 mg.

In some embodiments, the methods comprise a single dosage or administration (e.g., as a single injection or deposition). Alternatively, the methods comprise administration once daily, twice daily, three times daily or four times daily to a subject in need thereof for a period of from about 2 to about 28 days, or from about 7 to about 10 days, or from about 7 to about 15 days, or longer. In some embodiments, the methods comprise chronic administration. In yet other embodiments, the methods comprise administration over the course of several weeks, months, years, or decades. In still other embodiments, the methods comprise administration over the course of several weeks. In still other embodiments, the methods comprise administration over the course of several months. In still other embodiments, the methods comprise administration over the course of several years. In still other embodiments, the methods comprise administration over the course of several decades.

The dosage administered can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. These are all readily determined and may be used by the skilled artisan to adjust or titrate dosages and/or dosing regimens.

The precise dose to be employed in the compositions will also depend on the route of administration, and should be decided according to the judgment of the practitioner and each subject's circumstances. In specific embodiments of the disclosure, suitable dose ranges for oral administration of the compounds of the disclosure are generally about 1 mg/day to about 1000 mg/day. In some embodiments, the oral dose is about 1 mg/day to about 800 mg/day. In some embodiments, the oral dose is about 1 mg/day to about 500 mg/day. In some embodiments, the oral dose is about 1 mg/day to about 250 mg/day. In some embodiments, the oral dose is about 1 mg/day to about 100 mg/day. In some embodiments, the oral dose is about 5 mg/day to about 50 mg/day. In some embodiments, the oral dose is about 5 mg/day. In some embodiments, the oral dose is about 10 mg/day. In some embodiments, the oral dose is about 20 mg/day. In some embodiments, the oral dose is about 30 mg/day. In some embodiments, the oral dose is about 40 mg/day. In some embodiments, the oral dose is about 50 mg/day. In some embodiments, the oral dose is about 60 mg/day. In some embodiments, the oral dose is about 70 mg/day. In some embodiments, the oral dose is about 100 mg/day. It will be recognized that any of the dosages listed herein may constitute an upper or lower dosage range, and may be combined with any other dosage to constitute a dosage range comprising an upper and lower limit.

### Compositions

Another aspect of the disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, topical, buccal, ocular, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral, subcutaneous or intravenous administration.

Exemplary pharmaceutical compositions of this disclosure may be used in the form of a pharmaceutical preparation, for example, in solid, semisolid or liquid form, which contains one or more of the compound of the disclosure, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

In some embodiments, pharmaceutically acceptable compositions can contain a disclosed compound and/or a pharmaceutically acceptable salt thereof at a concentration ranging from about 0.01 to about 2.0 wt %, such as 0.01 to about 1 wt % or about 0.05 to about 0.5 wt %. The composition can be formulated as a solution, suspension, ointment, or a capsule, and the like. The pharmaceutical composition can be prepared as an aqueous solution and can contain additional components, such as preservatives, buffers, tonicity agents, antioxidants, stabilizers, viscosity-modifying ingredients and the like.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

Pharmaceutically acceptable carriers are well-known to those skilled in the art, and include, e.g., adjuvants, diluents, excipients, fillers, lubricants and vehicles. In some embodiments, the carrier is a diluent, adjuvant, excipient, or vehicle. In some embodiments, the carrier is a diluent, adjuvant, or excipient. In some embodiments, the carrier is a diluent or adjuvant. In some embodiments, the carrier is an excipient. Often, the pharmaceutically acceptable carrier is chemically inert toward the active compounds and is non-toxic under the conditions of use. Examples of pharmaceutically acceptable carriers may include, e.g., water or saline solution, polymers such as polyethylene glycol, carbohydrates and derivatives thereof, oils, fatty acids, or alcohols. Non-limiting examples of oils as pharmaceutical carriers include oils of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in e.g., Remington's: The Science and Practice of Pharmacy, 22nd Ed. (Allen, Loyd V., Jr ed., Pharmaceutical Press (2012)); Modern Pharmaceutics, 5^{th} Ed. (Alexander T. Florence, Juergen Siepmann, CRC Press (2009)); Handbook of Pharmaceutical Excipients, 7^{th} Ed. (Rowe, Raymond C.; Sheskey, Paul J.; Cook, Walter G.; Fenton, Marian E. eds., Pharmaceutical Press (2012)) (each of which hereby incorporated by reference in its entirety).

In some embodiments, the compounds of the disclosure are formulated into pharmaceutical compositions for administration to subjects in a biologically compatible form suitable for administration in vivo. According to another aspect, the present disclosure provides a pharmaceutical composition comprising a disclosed compound in admixture with a pharmaceutically acceptable diluent and/or carrier. The pharmaceutically-acceptable carrier is “acceptable” in the sense of being compatible with the other ingredients of the composition and not deleterious to the recipient thereof. The pharmaceutically-acceptable carriers employed herein may be selected from various organic or inorganic materials that are used as materials for pharmaceutical formulations and which are incorporated as analgesic agents, buffers, binders, disintegrants, diluents, emulsifiers, excipients, extenders, glidants, solubilizers, stabilizers, suspending agents, tonicity agents, vehicles and viscosity-increasing agents. Pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc and water, among others. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

Surfactants such as, e.g., detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N⁺R′R″R′″R″″Y⁻, in which the R radicals are identical or different optionally hydroxylated hydrocarbon radicals and Y⁻ is an anion of a strong acid, such as halide, sulfate and sulfonate anions; cetyltrimethylammonium bromide is one of the cationic surfactants which can be used, amine salts of formula N⁺R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

When administered to a subject, the disclosed compound and pharmaceutically acceptable carriers can be sterile. Suitable pharmaceutical carriers may also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, and the like. The present compositions, if desired, may also contain minor amounts of wetting or emulsifying agents, or pH buffering agents.

The pharmaceutical formulations of the present disclosure are prepared by methods well-known in the pharmaceutical arts. Optionally, one or more accessory ingredients (e.g., buffers, flavoring agents, surface active agents, and the like) also are added. The choice of carrier is determined by the solubility and chemical nature of the compounds, chosen route of administration and standard pharmaceutical practice.

Additionally, the compounds and/or compositions of the present disclosure are administered to a human or animal subject by known procedures including oral administration, sublingual or buccal administration. In some embodiments, the compound and/or composition is administered orally.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

For oral administration, a formulation of the compounds of the disclosure may be presented in dosage forms such as capsules, tablets, powders, granules, or as a suspension or solution. Capsule formulations may be gelatin, soft-gel or solid. Tablets and capsule formulations may further contain one or more adjuvants, binders, diluents, disintegrants, excipients, fillers, or lubricants, each of which are known in the art. Examples of such include carbohydrates such as lactose or sucrose, dibasic calcium phosphate anhydrous, corn starch, mannitol, xylitol, cellulose or derivatives thereof, microcrystalline cellulose, gelatin, stearates, silicon dioxide, talc, sodium starch glycolate, acacia, flavoring agents, preservatives, buffering agents, disintegrants, and colorants. Orally administered compositions may contain one or more optional agents such as, e.g., sweetening agents such as fructose, aspartame or saccharin; flavoring agents such as peppermint, oil of wintergreen, or cherry; coloring agents; and preservative agents, to provide a pharmaceutically palatable preparation.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants. For example, crystalline forms provided herein may be milled to obtain a particular particle size, and in at least some embodiments, such crystalline forms may remain substantially stable upon milling.

For example, provided herein is a composition suitable for subcutaneous administration, comprising a suspension of the disclosed crystalline form. Subcutaneous administration can be advantageous over intravenous administration, which typically requires a doctor visit, and can be more painful and invasive. A typical dose of the crystalline compound, when administered to a patient, may be about 1 mg to about 8 mg of compound. In an embodiment, disclosed herein is a pharmaceutically acceptable composition formed from a disclosed crystalline form, e.g. by mixing a crystalline form with an excipient and/or a solvent.

In an embodiment, provided herein is a composition comprising a disclosed crystalline form suitable for subcutaneous administration at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.001 mg/kg to about 4 mg/kg, from about 0.1 mg/kg to about 10 mg/kg, from about 1 mg/kg to about 25 mg/kg, of subject body weight, administered daily, one or more times a day, every other day, every third or fourth day, every week, every two weeks, every three weeks, or every four weeks. In certain embodiments, the desired dosage may be delivered using multiple administrations (e.g., two, three, four, five, six, seven, eight, nine, or ten administrations). In certain embodiments, administration may occur once, twice, or thrice weekly.

In certain embodiments, treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a weight loss target, is achieved. A treatment regimen can include a corrective phase, during which dose sufficient to provide reduction of weight is administered, and can be followed by a maintenance phase, during which a e.g. lower dose sufficient to weight gain is administered. A suitable maintenance dose is likely to be found in the lower parts of the dose ranges provided herein, but corrective and maintenance doses can readily be established for individual subjects by those of skill in the art without undue experimentation, based on the disclosure herein. Maintenance doses can be employed to maintain body weight in subjects whose body weight has been previously controlled by other means, including diet and exercise, bariatric procedures such as bypass or banding surgeries, or treatments employing other pharmacological agents.

In certain embodiments, provided herein is a pharmaceutical composition comprising a crystalline form of compound I-1, or a solvate thereof, as described herein. In certain embodiments, a pharmaceutical composition provided herein comprises one or more pharmaceutically acceptable excipient, as described herein. In some embodiments, provided herein is an immediate-release capsule comprising a crystalline form (e.g., Form A) of compound I-1, or a solvate thereof. In some embodiments, an immediate-release capsule comprises about 10 mg of a crystalline form (e.g., Form A) of compound I-1, or a solvate thereof. In some embodiments, an immediate-release capsule comprises about 50 mg of a crystalline form (e.g., Form A) of compound I-1, or a solvate thereof. In some embodiments, an immediate-release capsule comprises about 100 mg of a crystalline form (e.g., Form A) of compound I-1, or a solvate thereof. In some embodiments, an immediate-release capsule comprises a powder blend intermediate, which can be produced by directly blending a crystalline form (e.g., Form A) of compound I-1, or a solvate thereof, with one or more pharmaceutically acceptable excipients.

### Kits

In one embodiment, a kit for treating treating or mitigating a contemplated disease of disorder is provided. For example, a disclosed kit comprises a disclosed crystalline compound, e.g. a crystalline form of a compound of Formula (I), disposed in an e.g. first container. In some embodiments, a kit may further include a pharmaceutically acceptable excipient, disposed in e.g a second container. Such contemplated kits may include written instructions describing preparation of a pharmaceutical composition suitable for administration to a patient from the crystalline form. For example, the written instructions may describe preparing a pharmaceutically acceptable form for patient administration by e.g. mixing an excipient and a crystalline compound disclosed herein. Disclosed kits may further comprise written instructions describing how to administer the resulting composition to the patient.

### Processes

In some embodiments, a process for preparing a disclosed, crystalline form of a compound of Formula (I), e.g., Compound I-1, is contemplated herein, comprising: a) preparing a solution of Compound I-1 in a solvent comprising at least one of EtOH, ACN, MEK, EtOAc, IPAc, IPA, me-THF, MtBE, Toluene, 1,4 dioxane, heptane, and water; b) heating the solution to completely dissolve the Compound I-1; c) adjusting the temperature so that solid precipitates out of the solution; and d) isolating the crystalline form of Compound I-1.

In some embodiments, the solvent is EtOH. In some embodiments, the solvent comprises ACN. In some embodiments, the solvent comprises MEK. In some embodiments, the solvent comprises EtOAc. In some embodiments, the solvent comprises IPAc. In some embodiments, the solvent comprises IPA. In some embodiments, the solvent comprises me-THF. In some embodiments, the solvent comprises MtBE. In some embodiments, the solvent comprises Toluene. In some embodiments, the solvent comprises 1,4 dioxane. In some embodiments, the solvent comprises heptane. In some embodiments, the solvent comprises IPA and heptane. In some embodiments, the solvent comprises MEK and heptane. In some embodiments, heating the solution comprises heating the solution to about 50° C. In some embodiments, adjusting the temperature comprises cooling the solution to about 5° C.

### Deuterated Analogues

It will be appreciated by one of skill in the art, that deuterated analogues of compounds of Formula (I), may be made with deuterated starting materials using the synthetic processes described herein. As used herein, “deuterated analogues” of a formula means compounds having a structure of said formula except for the structure has at least one replacement of a hydrogen with a deuterium.

### EXAMPLES

The compounds described herein can be prepared in a number of ways based on the teachings contained herein and synthetic procedures known in the art. The following non-limiting examples illustrate the disclosures herein.

### Methods

X-ray Powder Diffraction (XRPD): X-Ray Powder Diffraction patterns were collected on a Bruker AXS D8 Advance diffractometer or a PANalytical Empyrean diffractometer.

Some XRPD diffractograms were collected on a Bruker D8 diffractometer using Cu Kα radiation (40 kV, 40 mA) in reflection geometry and a 0-20 goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector. The software used for data collection was Diffrac Plus XRD Commander and data analysis was HighScore Plus. Samples were run under ambient conditions as flat plate specimens using powder as received. The sample was prepared on a polished, zero-background (510) silicon wafer by gently pressing onto the flat surface or packed into a cut cavity. The sample was rotated in its own plane.

The details of the standard data collection method are:
Angular range: 2 to 42° 2θ
Step size: 0.05° 2θ
Collection time: 0.5 s/step (total collection time: 6.40 min)

Some XRPD diffractograms were collected on a PANalytical Empyrean diffractometer using Cu Kα radiation (45 kV, 40 mA) in transmission geometry. A 0.5° slit, 4 mm mask and 0.04 rad Soller slits with a focusing mirror were used on the incident beam. A PIXcel^{3D} detector, placed on the diffracted beam, was fitted with a receiving slit and 0.04 rad Soller slits. The software used for data collection was X'Pert Data Collector using X'Pert Operator Interface. The data were analysed and presented using HighScore Plus. Samples were prepared and analyzed in a metal 96 well-plate in transmission mode. X-ray transparent film was used between the metal sheets on the metal well-plate and powders (approximately 1-2 mg) were used as received.

The details of the standard screening data collection method are:
Angular range: 2.5 to 32.0° 2θ
Step size: 0.0130° 2θ
Collection time: 12.75 s/step (total collection time of 2.07 min)

Nuclear Magnetic Resonance (NMR) ¹H NMR spectra were collected on a Bruker 400 MHz instrument equipped with an auto-sampler and controlled by a Avance NEO nanobay console. Samples were prepared in DMSO-d₆ solvent, unless otherwise stated. Automated experiments were acquired using ICON-NMR configuration within Topspin software, using standard Bruker-loaded experiments (¹H). Off-line analysis was performed using ACD Spectrus Processor.

Differential Scanning Calorimetry (DSC): DSC data were collected on a TA Instruments Q2000 equipped with a 50 position auto-sampler. Typically, 0.5-3 mg of each sample, in a pin-holed aluminium pan, was heated at 10° C./min from 25° C. to 225° C. A purge of dry nitrogen at 50 ml/min was maintained over the sample. Modulated temperature DSC (MDSC) was carried out using an underlying heating rate of 2° C./min and temperature modulation parameters of ±0.636° C. (amplitude) every 60 seconds (period). The instrument control software was Advantage for Q Series and Thermal Advantage and the data were analyzed using Universal Analysis or TRIOS.

Thermal Gravimetric Analysis (TGA): TGA data were collected on a TA Instruments Q500 TGA, equipped with a 16 position autosampler. Typically, 5-10 mg of each sample was loaded onto a pre-tared aluminium DSC pan and heated at 10° C./min from ambient temperature to 350° C. A nitrogen purge at 60 ml/min was maintained over the sample. The instrument control software was Advantage for Q Series and Thermal Advantage and the data were analyzed using Universal Analysis or TRIOS.

Ion Chromatoaraphy (IC): Data were collected on a Metrohm 930 Compact IC Flex with 858 Professional autosampler and 800 Dosino dosage unit monitor, using IC MagicNet software. Accurately weighed samples were prepared as stock solutions in a suitable solvent. Quantification was achieved by comparison with standard solutions of known concentration of the ion being analysed. Analyses were performed in duplicate and an average of the values is given unless otherwise stated. Method for cation chromatography:

| Parameter | Value |
|---|---|
| Type of method | Cation exchange |
| Column | Metrosep C 4 - 250 (4.0 × 250 mm) |
| Column Temperature (° C.) | Ambient |
| Injection (μl) | Various |
| Detection | Conductivity detector |
| Flow Rate (ml/min) | 0.9 |
| Eluent | 1.7 mM nitric acid |
| | 0.7 mM dipicolinic acid in a 5% acetone |
| | aqueous solution. |

### Method for Anion Chromatography:

| Parameter | Value |
|---|---|
| Type of method | Anion exchange |
| Column | Metrosep A Supp 5 - 150 (4.0 × 150 mm) |
| Column Temperature (° C.) | Ambient |
| Injection (μl) | Various |
| Detection | Conductivity detector |
| Flow Rate (ml/min) | 0.7 |
| Eluent | 3.2 mM sodium carbonate |
| | 1.0 mM sodium hydrogen carbonate in a |
| | 5% acetone aqueous solution. |

### Materials

### FaSSGF (Fasted State Simulated Gastric Fluid)

Base Buffer (FaSSGF), pH 1.6—Sodium chloride (2.03 g) was dissolved in Ca. 800 ml of deionised water in a 1 L volumetric flask. This solution was mixed thoroughly and then the pH adjusted to 1.6±0.05 with concentrated hydrochloric acid. The solution was then made to volume with deionised water and the pH recorded (pH 1.6).

FaSSGF Media—Phares SIF (simulated intestinal fluid) powder (0.06 g) was dissolved in Ca. 800 ml of base buffer (2.1.1) in a 1 L volumetric flask. This solution was mixed thoroughly and then made to volume with the same base buffer. The final pH was recorded (pH 1.7)

### FeSSIF (Fed State Simulated Intestinal Fluid)

Acetate Base Buffer (FeSSIF), pH 5.0—Sodium hydroxide (11.90 g), acetic acid (8.25 ml) and sodium chloride (3.96 g) were dissolved in Ca. 800 ml of deionised water in a 1 L volumetric flask. This solution was mixed thoroughly and then the pH adjusted to 5.0±0.05 with 0.5M hydrochloric acid. The solution was then made to volume with deionised water and the pH recorded (pH 5.0)

FeSSIF Media—Phares SIF (simulated intestinal fluid) powder (1.12 g) was dissolved in Ca. 80 ml of base buffer (2.2.1) in a 100 ml volumetric flask. This solution was mixed thoroughly and then made to volume with the same base buffer. The final pH was recorded (pH 4.8)

### FaSSIF (Fasted State Simulated Intestinal Fluid)

Phosphate Base Buffer (FeSSIF), pH 6.5—Sodium hydroxide (0.41 g), sodium phosphate monobasic (3.95 g) and sodium chloride (6.49 g) were dissolved in Ca. 800 ml of deionised water in a 1 L volumetric flask. This solution was mixed thoroughly and then the pH adjusted to 6.5 f 0.05 with 0.5M hydrochloric acid. The solution was then made to volume with deionised water and the pH recorded (pH 6.5)

FaSSIF Media—Phares SIF (simulated intestinal fluid) powder (0.23 g) was dissolved in Ca. 80 ml of base buffer ([00300]) in a 100 ml volumetric flask. This solution was mixed thoroughly and then made to volume with the same base buffer. The final pH was recorded (pH 6.3)

DI Water (deionized water)—The pH was recorded (pH 6.1)

### Example 1—Synthesis of (3R,5R)-5-(5-(3-(2,2-difluoroethoxy)-1-methyl-1H-pyrazole-5-carboxamido)-1H-pyrazol-3-yl)tetrahydrofuran-3-yl bicyclo[1.1.1]pentan-1-ylcarbamate (Compound I-1)

### Step 1. Preparation of Compound 3

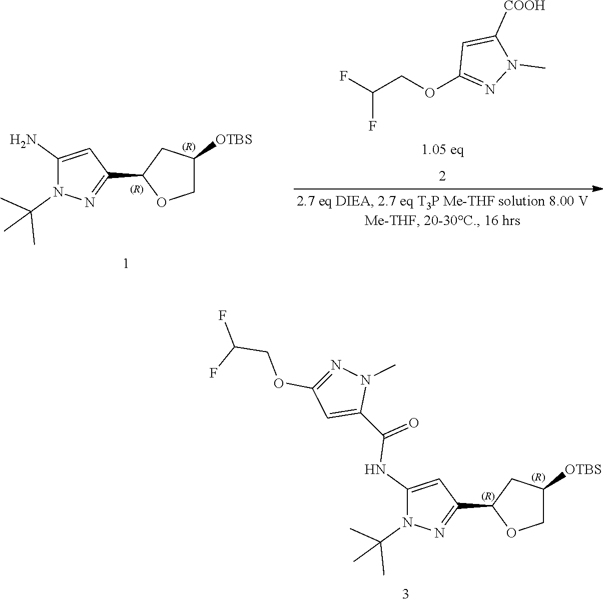

To a mixture of 1-(tert-butyl)-3-((2R,4R)-4-((tert-butyldimethylsilyl)oxy)tetrahydrofuran-2-yl)-1H-pyrazol-5-amine (compound 1, 10 g) in 2-methyltetrahydrofuran (69 g, 6.9 equiv.) was added 3-(2,2-difluoroethoxy)-1-methyl-1H-pyrazole-5-carboxylic acid (compound 2, 6.4 g, 1.05 equiv.) and DIPEA (9.6 g, 2.7 equiv) under a nitrogen atmosphere. The mixture temperature was adjusted to 0° C., and propylphosphonic anhydride solution in 2-MeTHF (26 g net, 2.7 equiv., 8 vol) was added. The mixture was adjusted to 25° C. and allowed to stir for 16 h at around 20-30° C. The mixture was cooled to 0° C. and was charged with water (100 g). The mixture temperature was adjusted to 15° C. and was allowed to stir at 10-20° C. for 30 min, and then the mixture was allowed stand without stirring for 30 min. The organic layer was washed with 7% aq. NaHCO₃ solution (50 g, 2 times), and concentrated in vacuo to obtain compound 3 (13 g, 84% yield).

### Step 2. Preparation of Compound 4

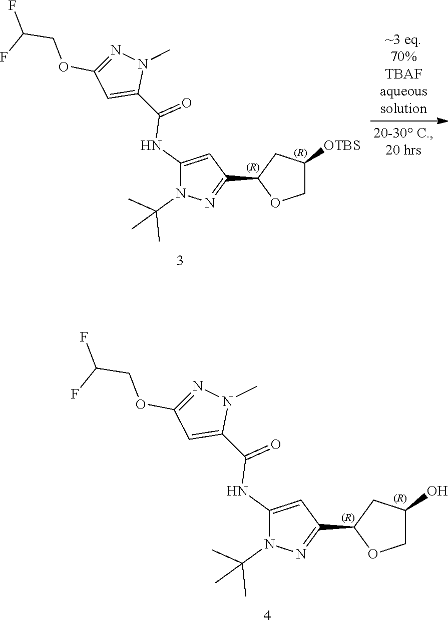

A reactor was charged with N-(1-(tert-butyl)-3-((2R,4R)-4-((tert-butyldimethylsilyl)oxy)tetrahydrofuran-2-yl)-1H-pyrazol-5-yl)-3-(2,2-difluoroethoxy)-1-methyl-1H-pyrazole-5-carboxamide (compound 3, 100 g), 2-methyltetrahydrofuran (520 g, 6 vol), and 70% aq. tetrabutylammonium fluoride (150 g, 3.0 equiv) and the contents were stirred at 25° C. for 15 hrs. After, the mixture was charged with 7% aq. NaHCO₃ (500 g, 5 vol) and was allowed to stir at 25° C. for 30 min and then allowed to stand without stirring for 30 min. The aqueous layer was removed, and to the organic layer was added water (500 g, 5 vol) and then 1M aq. HCl (90 g) to adjust the pH to ˜6. The resulting mixture was stirred at 25° C. for 30 min and then allowed to stand without stirring for 30 min. The aqueous layer was removed, and to the organic layer was added 7% aq. NaHCO₃ (500 g, 5 vol) and was allowed to stir at 25° C. for 30 min and then allowed to stand without stirring for 30 min. The organic layer was separated and concentrated in vacuo, and redissolved in 2-methyltetrahydrofuran (100 g, 3 times). The concentrate was then charged with n-heptane (136 g, 2 vol). The mixture was charged with seed crystal and stirred for 2 hrs at 25° C. Another portion of n-heptane (408 g, 6 vol) was added, and the mixture was stirred for 2 hrs at 25° C. The mixture temperature was then adjusted to 0° C. and allowed to stir for 10 hrs. The solid precipitate was filtered, and the resulting wet cake was washed with n-heptane (66 g). The wet cake was dried by vacuum at 45° C. for 16 hrs to obtain N-(1-(tert-butyl)-3-((2R,4R)-4-hydroxytetrahydrofuran-2-yl)-1H-pyrazol-5-yl)-3-(2,2-difluoroethoxy)-1-methyl-1H-pyrazole-5-carboxamide (compound 4, 66 g, 84% yield).

### Step 3. Preparation of Compound 5

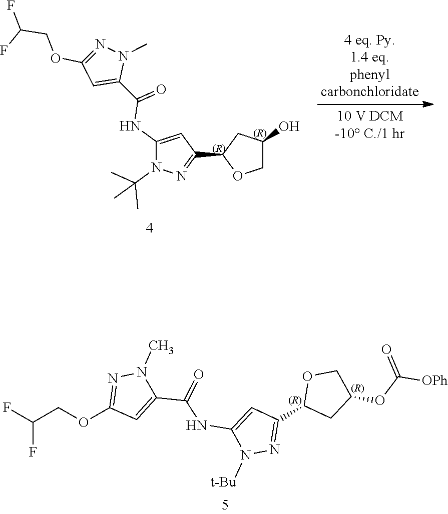

A reactor was charged with N-(1-(tert-butyl)-3-((2R,4R)-4-hydroxytetrahydrofuran-2-yl)-1H-pyrazol-5-yl)-3-(2,2-difluoroethoxy)-1-methyl-1H-pyrazole-5-carboxamide (compound 4,100 g) and dichloromethane (1300 g, 10 vol) and the temperature was adjusted to −10° C. The mixture was then charged with pyridine (75.6 g, 4 equiv.) and phenyl chloroformate (53 g, 1.4 equiv.). The mixture was stirred for 1 h at −10° C. The mixture was then charged with 1 M aq. HCl (400 g, 3.9 vol) and then allowed to stir for 30 min at 20° C. The organic layer was separated, charged with 7% aq. NaHCO₃ (500 g, 4.9 vol). The mixture was stirred for 30 min and then allowed to stand without stirring for 30 min. The organic layer containing compound 5 was concentrated to reduce volume, and the solution was used directly in the next step.

### Step 4. Preparation of Compound 6

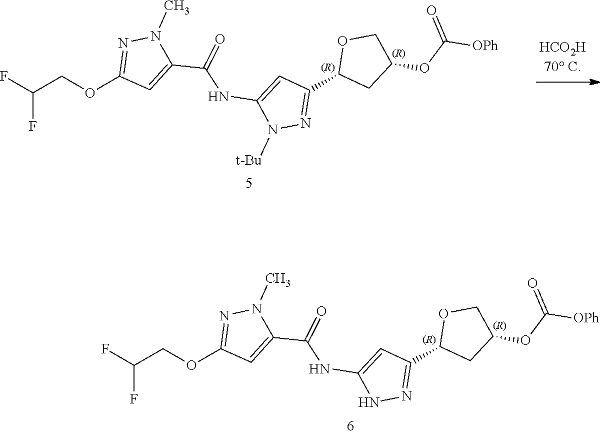

Under a nitrogen atmosphere, a reactor was charged with the solution of compound 5 from step 3 (100 g) and formic acid (671 g). The mixture was concentrated under vacuum for 2 hrs, then stirred for 16 hrs at 70° C. The temperature was then adjusted to 25° C. and the mixture was charged with dichloromethane (1330 g) and water (1000 g). The mixture was stirred for 1 hr and allowed to stand without stirring for 1 hr. The organic layer was washed with water (10 vol, 2 times), and the organic layer was concentrated under vacuum. The concentrate was charged with MTBE and seed crystal. The mixture was stirred for 1 hr at 25° C. and then −5° C. for 16 hrs. The precipitate was filtered, and the resulting wet cake was washed with pre-cold MTBE (1-2 times). The wet cake was then dried at 50-60° C. for 16 hrs to obtain compound 6.

### Step 5. Preparation of Compound I-1

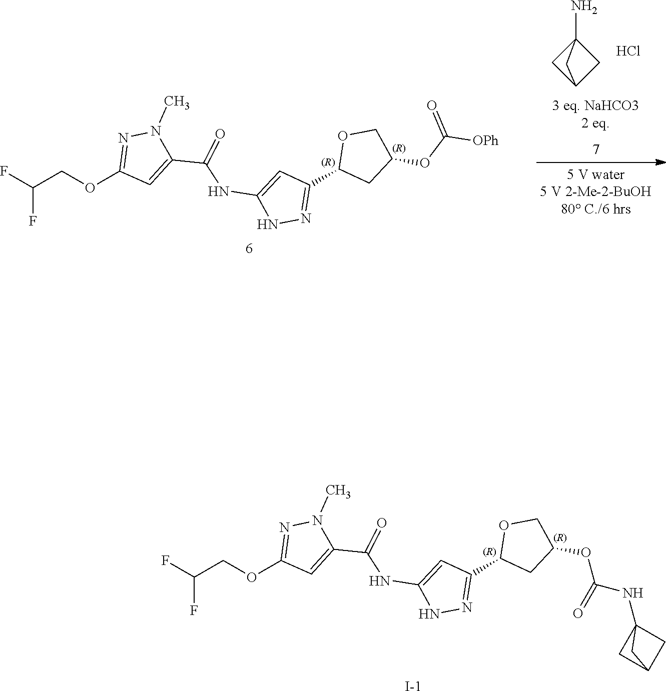

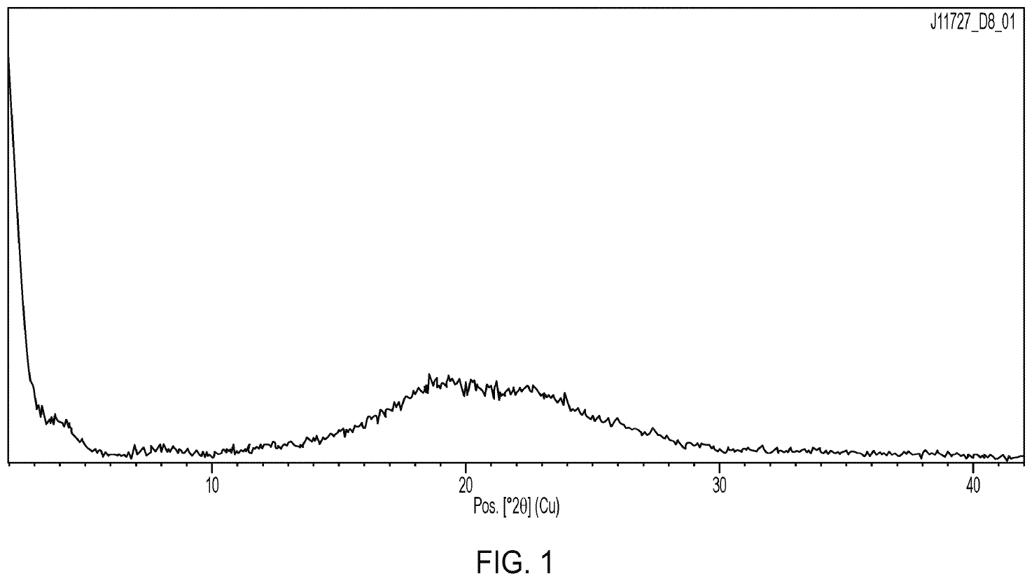

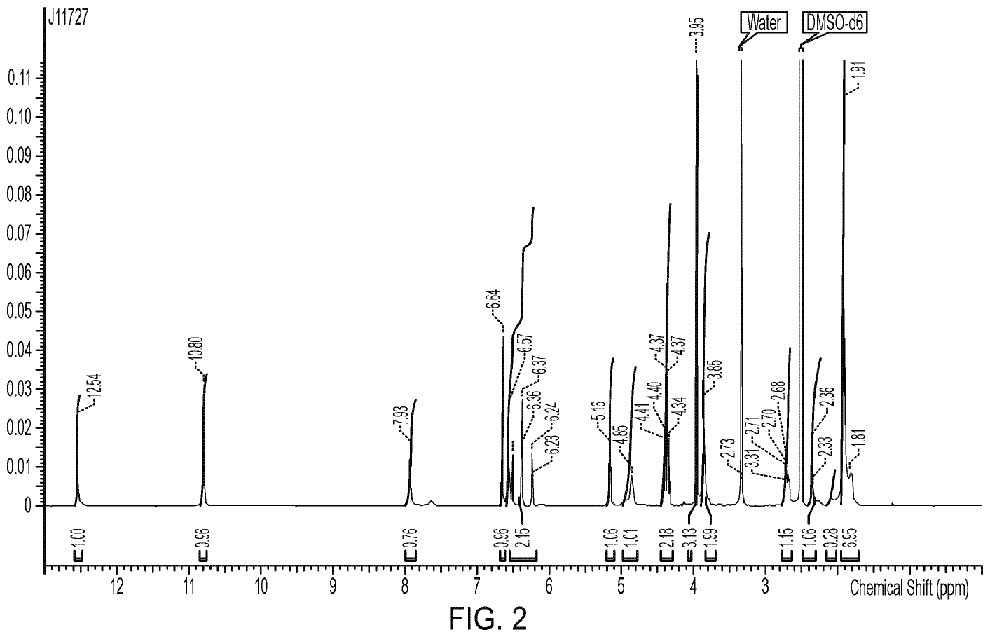

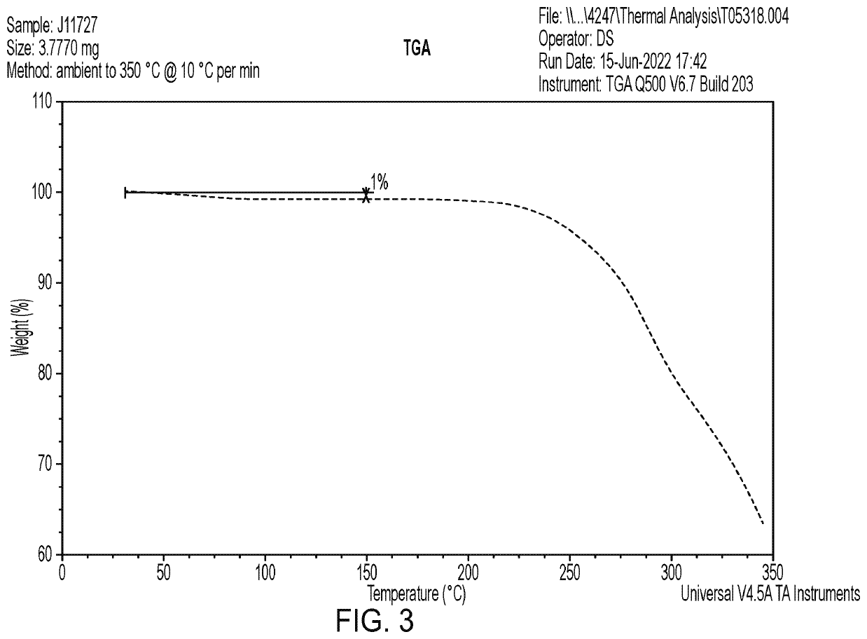

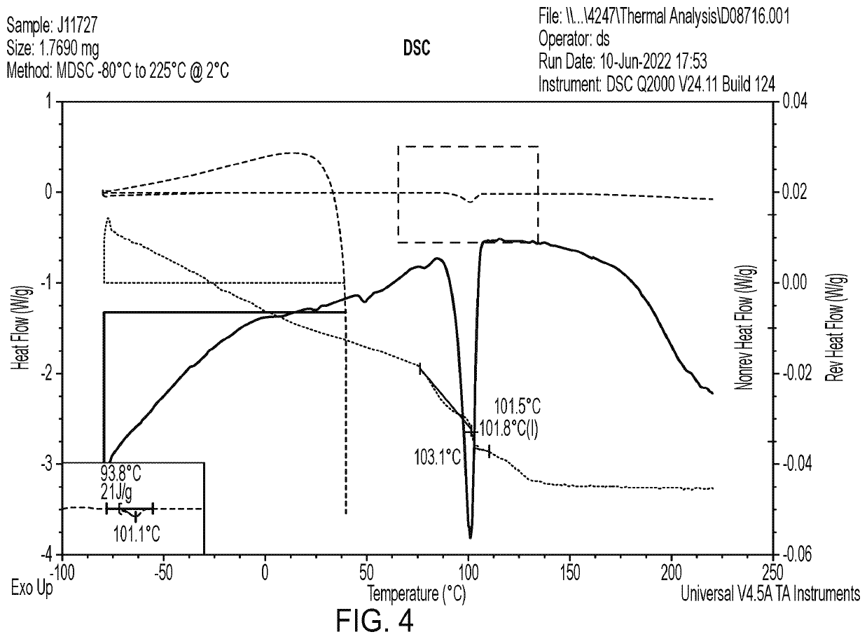

Under a nitrogen atmosphere, a reactor was charged with Na₂CO₃ (52.8 g) and water (500 g) at 25° C. and stirred for ˜30 min until clear. The mixture was charged with 2-methyl-2-butanol (405 g, 5 vol), bicyclo[1.1.1]pentan-1-amine hydrochloride (compound 7) (50 g, 2 equiv.), and (3R,5R)-5-(5-(3-(2,2-difluoroethoxy)-1-methyl-1H-pyrazole-5-carboxamido)-1H-pyrazol-3-yl)tetrahydrofuran-3-yl phenyl carbonate (compound 6) (100 g), and then heated to 80° C. for 6 hrs. The mixture was cooled to 25° C. was charged with 2-methyltetrahydrofuran (430 g) and stirred for 1 hr and allowed to stand for 1 hr without stirring. The organic layer was separated and charged with 3% aq. NaOH (1000 g), stirred for 1 hr and allowed to stand without stirring for 1 hr. The organic phase was concentrated and dried to yield amorphous compound I-1. The resulting X-ray diffraction pattern demonstrated compound I-1 was amorphous (FIG. 1). ¹H NMR spectrum: FIG. 2. TGA pattern: FIG. 3. DSC pattern: FIG. 4.

Crystallization: The amorphous compound I-1 was then subjected to three cycles of charging with methyl ethyl ketone (6 vol) and concentrating to 1 volume. The resulting concentrate was charged with methyl ethyl ketone (6 vol) and heated to 50° C. and stirred until mostly clear. The solution was filtered and charged with heptane (610 g) and stirred at 50° C. for 6 hrs. The solution was then cooled to −5° C. for 6 hrs. The precipitate was filtered, and the wet cake was washed with pre-cold heptane (2 times). The wet cake was then dried at 50° C. for 18 hrs to yield crystalline compound I-1 consistent with Form A by XRPD.

### Example 2—Polymorph Screen of (3R,5R)-5-(5-(3-(2,2-difluoroethoxy)-1-methyl-1H-pyrazole-5-carboxamido)-1H-pyrazol-3-yl)tetrahydrofuran-3-yl bicyclo[1.1.1]pentan-1-ylcarbamate (Compound I-1)

Polymorph screening of the Compound I-1 (also referred to as (3R,5R)-5-(5-(3-(2,2-difluoroethoxy)-1-methyl-1H-pyrazole-5-carboxamido)-1H-pyrazol-3-yl)tetrahydrofuran-3-yl bicyclo[1.1.1]pentan-1-ylcarbamate) was performed in different solvents by a temperature cycling method. If no suspended solids were observed when the system was cooled to 25° C., then the solution was evaporated. Details of operation procedures were listed as below:

Conversion of Compound I-1 was studied under anhydrous and aqueous conditions at 50° C. Table 1 summarizes the results of Form A and Form B conversion investigated in these studies. Compound I-1 (ca. 100 mg, amorphous) and appropriate solvent (10 vol) were charged into separate 1.5 ml clear glass vials, heated at 0.5° C./min (over 1 hour) to 50° c., stirred at 50° c. for 16 hours and cooled at −0.5° C./min (over 1 hour). After this time, products were isolated by centrifuging at 10,000 RPM for 10 minutes, dried under reduced pressure at 40° c. and analyzed by XRPD, HPLC and ¹H NMR. If a new XRPD pattern was identified, the dry solids with new XRPD patterns were also characterized by DSC and TGA.

A summary of the solvents examined can be found in Table 1:

TABLE 1

| Solvent | XRPD |
|---|---|
| IPA/Hept (1:5) | Form A |
| Isopropyl acetate | Form B |

### Compound I-1 Form A

After polymorph screening experiments described above, obtained solids all showed XRPD patterns, including Form A.

The XRPD of Form A of Compound I-1 is shown in FIG. 5. Table 2 below sets out the X-Ray diffraction peaks observed for Form A of Compound I-1, wherein each value is in degrees 2θ:

TABLE 2
XRPD Peak list of Compound I-1 Form A

| Angle (2θ)° ¹ | Rel. Intensity % |
|---|---|
| 5.2 | 100.0 |
| 5.8 | 6.3 |
| 9.6 | 10.0 |
| 11.6 | 14.2 |
| 13.8 | 5.1 |
| 14.2 | 9.6 |
| 15.6 | 11.6 |
| 18.5 | 23.9 |
| 19.2 | 8.6 |
| 20.8 | 7.3 |
| 21.2 | 9.1 |
| 21.6 | 4.9 |

¹ In this and all subsequent tables, the position 2θ is within ± 0.2.

### Compound I-1 Form B

After polymorph screening experiments described above, obtained solids all showed XRPD patterns, including Form B.

The XRPD of Form B of Compound I-1 is shown in FIG. 6. Table 3 below sets out the X-Ray diffraction peaks observed for Form B of Compound I-1, wherein each value is in degrees 2θ:

TABLE 3
XRPD Peak list of Compound I-1 Form B

| Angle (2θ)° ¹ | Relative Intensity % |
|---|---|
| 4.2 | 100 |
| 7.3 | 14.8 |
| 8.4 | 8.6 |
| 11.3 | 14.2 |
| 17.3 | 8.5 |
| 18.2 | 11.2 |
| 18.8 | 24.6 |
| 19.7 | 30.1 |
| 21.3 | 10.8 |
| 22.6 | 58 |
| 23.4 | 16.7 |

¹ In this and all subsequent tables, the position 2θ is within ± 0.2.

### Example 3—Cocrystal screening studies on of (3R,5R-5-(5-(3-(2,2-difluoroethoxy)-1-methyl-1H-pyrazole-5-carboxamido)-1H-pyrazol-3-yl)tetrahydrofuran-3-yl bicyclo[1.1.1]pentan-1-ylcarbamate (Compound I-1)

Amorphous Compound I-1 and 1 equiv. of the selected coformers (Error! Reference source not found.) in HPLC vials were treated with IPA (2 vol, 60 μl) at RT. Steel grinding balls (2×3 mm) were added before samples were subjected to grinding for 2 hours at 500 rpm in a planetary mill. Observations were taken, and any solids were analysed by XRPD.

Any amorphous, free form patterns or solutions after grinding were treated with heptane:IPA (5:1, 5 vol, 150 μl) and subjected to maturation cycling between 25° C./50° C. (4 hours per cycle) for 48 hours. Observations were recorded, and wet solids were analysed by XRPD.

From the screening procedures involving liquid assisted grinding and maturation, 10 cocrystals and 11 unique cocrystal XRPD patterns were observed as shown in Table 4.

TABLE 4
Cocrystal Screening

| | XRPD after | XRPD after |
|---|---|---|
| Coformer | grinding | maturation cycle |
| Saccharin | — | — |
| Succinic acid | Compound I-2 | — |
| | (Form A) | |
| Urea | — | Compound I-7 |
| | | (FormA) |
| Nicotinamide | — | Compound I-6 |
| | | (Form A) |
| L-malic acid | Compound I-3 | — |
| | (Form A) | |
| L-lactic acid | Compound I-4 | — |
| | (Form A) | |
| Benzoic acid | Compound I-5 | — |
| | (Form A) | |
| Citric acid | — | — |
| L-tartaric | Compound I-8 | — |
| acid | (Form A) | |
| Sorbic acid | — | — |
| Sulfacetamide | — | — |
| Ketoglutaric | — | — |
| acid | | |
| Maltol | — | Compound I-9 |
| | | (Form A) |
| L-proline | — | Compound I-10 |
| | | (Form A) |
| p- | — | — |
| toluenesulfonic | | |
| acid | | |
| Salicylic | — | — |
| acid | | |
| 4- | Compound I-11 | — |
| aminobenzoic | (Form A, Form B) | |
| acid | | |
| 2,5- | — | — |
| dihydroxybenzoic | | |
| acid | | |

“—” indicates that a cocrystal was not formed.

### Compound I-2

After cocrystal polymorph screening experiments described above, obtained solids all showed XRPD patterns. The XRPD of Form A of Compound I-2 is shown in FIG. 7. Table 5 below sets out the X-Ray diffraction peaks observed for Form A of Compound I-2, wherein each value is in degrees 2θ:

TABLE 5
XRPD Peak list of Compound I-2 Form A

| Angle (2-Theta °) ¹ | Intensity (%) |
|---|---|
| 5.3 | 100.0 |
| 6.6 | 9.8 |
| 8.0 | 7.3 |
| 10.0 | 12.9 |
| 10.6 | 43.5 |
| 12.2 | 24.5 |
| 12.4 | 21.0 |
| 13.3 | 3.9 |
| 14.5 | 4.3 |
| 16.1 | 6.8 |
| 17.1 | 4.0 |
| 17.3 | 4.8 |
| 17.7 | 4.2 |
| 18.1 | 7.1 |
| 18.4 | 22.7 |
| 19.6 | 61.8 |
| 19.9 | 11.3 |
| 20.6 | 6.1 |
| 21.3 | 14.6 |
| 22.0 | 20.6 |
| 22.2 | 5.6 |
| 23.0 | 9.5 |
| 23.3 | 4.6 |
| 23.7 | 5.0 |
| 23.9 | 5.3 |
| 24.4 | 13.3 |
| 25.0 | 13.3 |
| 25.7 | 8.0 |
| 26.1 | 6.5 |
| 26.8 | 10.8 |
| 27.1 | 12.7 |
| 27.5 | 5.2 |
| 27.8 | 8.8 |
| 29.3 | 7.2 |

¹ In this and all subsequent tables, the position 2θ is within ± 0.2.

¹H NMR analysis indicated was Compound I-2 was consistent with a succinic acid cocrystal, with residual (<0.1 mol equiv.) IPA present. See FIG. 8. The sample was confirmed to contain ca. 1 mol equiv. of succinic acid.

The TGA thermogram of the material showed a small mass loss of 2 wt % (equiv. to 0.2 mol equiv. of IPA) before decomposition (above 200° C.). See FIG. 9. The DSC trace showed a double endotherm with an onset of 140.4° C. (80 J/g). See FIG. 9.

### Compound I-3

After cocrystal polymorph screening experiments described above, obtained solids all showed XRPD patterns. The XRPD of Form A of Compound I-3 is shown in FIG. 10. Table 6 below sets out the X-Ray diffraction peaks observed for Form A of Compound I-3, wherein each value is in degrees 2θ:

TABLE 6
XRPD peak list of Compound I-3 Form A

| Angle (2-Theta °) ¹ | Intensity (%) |
|---|---|
| 5.3 | 100.0 |
| 5.8 | 27.4 |
| 6.9 | 6.8 |
| 8.8 | 7.4 |
| 10.7 | 8.0 |
| 10.8 | 19.1 |
| 11.7 | 2.1 |
| 13.3 | 62.6 |
| 13.8 | 3.8 |
| 15.7 | 4.9 |
| 16.0 | 3.0 |
| 17.6 | 60.6 |
| 18.3 | 51.0 |
| 18.8 | 17.2 |
| 19.8 | 19.0 |
| 20.3 | 32.7 |
| 20.8 | 85.3 |
| 21.4 | 24.2 |
| 22.1 | 8.3 |
| 22.9 | 11.4 |
| 23.6 | 36.8 |
| 23.9 | 8.6 |
| 24.3 | 24.6 |
| 24.8 | 2.5 |
| 25.2 | 3.0 |
| 25.4 | 3.3 |
| 25.8 | 11.8 |
| 26.1 | 3.6 |
| 26.9 | 16.3 |
| 28.2 | 15.3 |
| 28.9 | 23.9 |
| 29.5 | 4.8 |
| 31.8 | 9.7 |

¹ In this and all subsequent tables, the position 2θ is within ± 0.2.

¹H NMR analysis indicated Compound I-3 was consistent with a malic acid cocrystal, with residual (<0.1 mol equiv.) IPA present. See FIG. 11. The sample was confirmed to contain ca. 1 mol equiv. of malic acid.

The TGA thermogram of the material showed a small mass loss of 2 wt % (equiv. to 0.2 mol equiv. of IPA) before decomposition (above 200° C.). See FIG. 12. The DSC trace showed a single sharp endotherm with an onset of 153.1° C. (112 J/g). See FIG. 12.

### Compound I-4

After cocrystal polymorph screening experiments described above, obtained solids all showed XRPD patterns. The XRPD of Form A of Compound I-4 is shown in FIG. 13. Table 7 below sets out the X-Ray diffraction peaks observed for Form A of Compound I-4, wherein each value is in degrees 2θ:

TABLE 7
XRPD peak list of Compound I-4 Form A

| Angle (2-Theta °) ¹ | Intensity (%) |
|---|---|
| 5.4 | 88.6 |
| 6.2 | 27.8 |
| 6.7 | 6.8 |
| 10.6 | 23.3 |
| 10.8 | 6.1 |
| 13.5 | 2.8 |
| 13.6 | 3.3 |
| 14.2 | 21.9 |
| 15.4 | 7.4 |
| 16.3 | 4.1 |
| 17.4 | 24.1 |
| 17.5 | 17.8 |
| 18.2 | 17.6 |
| 18.8 | 76.1 |
| 19.1 | 45.0 |
| 20.1 | 11.9 |
| 20.4 | 100.0 |
| 21.6 | 6.4 |
| 21.8 | 12.6 |
| 22.3 | 9.8 |
| 23.1 | 7.9 |
| 23.4 | 6.1 |
| 23.5 | 6.9 |
| 24.0 | 20.9 |
| 24.8 | 74.0 |
| 26.0 | 10.4 |
| 26.7 | 8.8 |
| 28.8 | 23.9 |
| 29.4 | 3.7 |
| 29.6 | 8.3 |
| 29.7 | 6.9 |
| 29.9 | 8.2 |
| 31.9 | 6.1 |

¹ In this and all subsequent tables, the position 2θ is within ± 0.2.

¹H NMR analysis indicated Compound I-4 was consistent with a lactic acid cocrystal, with residual IPA and lactic acid present. See FIG. 14.

The TGA thermogram of the material showed a small mass loss of 4 wt % (equiv. to 0.4 mol equiv. of IPA) before decomposition (above 190° C.). See FIG. 15. The DSC trace showed a single sharp endotherm with an onset of 135.5° C. (70 J/g). See FIG. 15.

### Compound I-5

After cocrystal polymorph screening experiments described above, obtained solids all showed XRPD patterns. The XRPD of Form A of Compound I-5 is shown in FIG. 16. Table 8 below sets out the X-Ray diffraction peaks observed for Form A of Compound I-5, wherein each value is in degrees 2θ:

TABLE 8
XRPD peak list of benzoic acid Compound I-5 Form A

| Angle (2-Theta °) ¹ | Intensity (%) |
|---|---|
| 5.2 | 100.0 |
| 6.7 | 14.1 |
| 8.5 | 16.0 |
| 8.9 | 2.9 |
| 10.2 | 7.2 |
| 11.0 | 5.8 |
| 11.8 | 15.1 |
| 13.5 | 3.3 |
| 13.7 | 3.6 |
| 15.3 | 12.3 |
| 16.5 | 3.5 |
| 16.6 | 3.8 |
| 16.7 | 3.3 |
| 17.2 | 19.1 |
| 17.8 | 22.7 |
| 18.0 | 25.5 |
| 18.1 | 26.4 |
| 19.2 | 15.2 |
| 19.6 | 12.5 |
| 19.9 | 19.6 |
| 20.2 | 10.6 |
| 20.5 | 13.1 |
| 21.3 | 23.5 |
| 22.1 | 32.3 |
| 22.8 | 11.0 |
| 23.1 | 10.9 |
| 23.4 | 4.9 |
| 23.6 | 5.6 |
| 24.0 | 15.5 |
| 24.3 | 7.0 |
| 24.5 | 7.1 |
| 25.0 | 9.4 |
| 26.9 | 3.6 |
| 27.3 | 2.1 |
| 28.3 | 2.4 |
| 28.5 | 2.5 |
| 28.7 | 3.9 |
| 28.9 | 3.7 |
| 30.5 | 3.2 |

¹ In this and all subsequent tables, the position 2θ is within ± 0.2.

¹H NMR analysis indicated Compound I-5 was consistent with a benzoic acid cocrystal, with residual (0.4 mol equiv.) IPA present. See FIG. 17. The sample was confirmed to contain ca. 1-1.5 mol equiv. of benzoic acid.

The TGA thermogram of the material showed a small mass loss of 1 wt % (equiv. to 0.1 mol equiv. of IPA) before decomposition (above 170° C.). See FIG. 18. The DSC trace showed a single sharp endotherm with an onset of 111.6° C. (60 J/g). See FIG. 18.

### Compound I-6

After cocrystal polymorph screening experiments described above, obtained solids all showed XRPD patterns. The XRPD of Form A of Compound I-6 is shown in FIG. 19. Table 9 below sets out the X-Ray diffraction peaks observed for Form A of Compound I-6, wherein each value is in degrees 2θ:

TABLE 9
XRPD peak list of nicotinamide Compound I-6 Form A

| Angle (2-Theta °) ¹ | Intensity (%) |
|---|---|
| 4.5 | 100.0 |
| 12.7 | 6.2 |
| 13.6 | 12.0 |
| 15.1 | 8.9 |
| 15.6 | 14.6 |
| 16.4 | 26.2 |
| 17.5 | 18.3 |
| 18.2 | 16.1 |
| 18.8 | 31.1 |
| 19.8 | 6.2 |
| 20.3 | 8.8 |
| 21.8 | 3.4 |
| 22.7 | 40.7 |
| 23.6 | 7.3 |
| 25.6 | 63.3 |
| 26.4 | 5.5 |
| 30.2 | 2.5 |

¹ In this and all subsequent tables, the position 2θ is within ± 0.2.

¹H NMR analysis indicated Compound I-6 was consistent with a nicotinamide cocrystal, with residual (0.4 mol equiv.) IPA present. See FIG. 20. The sample was confirmed to contain ca. 1 mol equiv. of nicotinamide.

The TGA thermogram of the material showed a small mass loss of 2 wt % (equiv. to 0.2 mol equiv. of IPA) before decomposition (above 220° C.). See FIG. 21. The DSC trace showed a single sharp endotherm with an onset of 146.3° C. (105 J/g). See FIG. 21.

### Compound I-7

After cocrystal polymorph screening experiments described above, obtained solids all showed XRPD patterns. The XRPD of Form A of Compound I-7 is shown in FIG. 22. Table 10 below sets out the X-Ray diffraction peaks observed for Form A of Compound I-7, wherein each value is in degrees 2θ:

TABLE 10
XRPD peak list of urea Compound I-7 Form A

| Angle (2-Theta °) ¹ | Intensity (%) |
|---|---|
| 4.0 | 39.8 |
| 5.8 | 36.7 |
| 8.0 | 8.9 |
| 9.0 | 24.5 |
| 9.4 | 3.2 |
| 11.7 | 9.8 |
| 12.0 | 6.8 |
| 12.7 | 9.4 |
| 13.4 | 8.2 |
| 14.7 | 6.3 |
| 15.1 | 12.7 |
| 16.0 | 2.2 |
| 16.6 | 3.0 |
| 17.1 | 37.4 |
| 17.6 | 100.0 |
| 18.1 | 25.3 |
| 18.9 | 5.9 |
| 19.2 | 2.7 |
| 20.6 | 12.3 |
| 21.4 | 22.8 |
| 21.8 | 10.1 |
| 22.2 | 12.7 |
| 22.9 | 43.1 |
| 23.4 | 2.5 |
| 23.9 | 8.0 |
| 24.5 | 8.0 |
| 25.6 | 4.7 |
| 26.1 | 2.8 |
| 26.8 | 12.3 |
| 27.0 | 6.9 |
| 27.3 | 5.1 |
| 28.6 | 4.6 |
| 29.4 | 2.9 |

¹ In this and all subsequent tables, the position 2θ is within ± 0.2.

### Compound I-8

After cocrystal polymorph screening experiments described above, obtained solids all showed XRPD patterns. The XRPD of Form A of Compound I-8 is shown in FIG. 23. Table 11 below sets out the X-Ray diffraction peaks observed for Form A of Compound I-8, wherein each value is in degrees 2θ:

TABLE 11
XRPD peak list of L-tartaric acid Compound I-8 Form A

| Angle (2-Theta °) ¹ | Intensity (%) |
|---|---|
| 5.3 | 100.0 |
| 10.1 | 15.7 |
| 10.6 | 32.6 |
| 11.5 | 5.9 |
| 12.4 | 10.1 |
| 14.7 | 17.1 |
| 16.0 | 15.0 |
| 16.5 | 14.8 |
| 17.2 | 27.0 |
| 17.6 | 9.0 |
| 18.2 | 17.0 |
| 18.6 | 26.7 |
| 18.8 | 48.0 |
| 19.3 | 21.4 |
| 19.5 | 26.2 |
| 20.1 | 21.7 |
| 21.0 | 8.4 |
| 21.3 | 10.4 |
| 22.2 | 17.1 |
| 22.5 | 24.8 |
| 22.9 | 38.7 |
| 24.6 | 15.8 |
| 24.9 | 31.3 |
| 25.3 | 8.6 |
| 25.7 | 6.2 |
| 25.8 | 5.9 |
| 26.8 | 7.7 |
| 26.9 | 2.4 |
| 28.5 | 5.4 |
| 29.1 | 15.8 |

¹ In this and all subsequent tables, the position 2θ is within ± 0.2.

### Compound I-9

After cocrystal polymorph screening experiments described above, obtained solids all showed XRPD patterns. The XRPD of Form A of Compound I-9 is shown in FIG. 24. Table 12 below sets out the X-Ray diffraction peaks observed for Form A of Compound I-9, wherein each value is in degrees 2θ:

TABLE 12
XRPD peak list of maltol Compound I-9 Form A

| Angle (2-Theta °) ¹ | Intensity (%) |
|---|---|
| 10.5 | 6.0 |
| 11.7 | 17.4 |
| 13.3 | 25.4 |
| 14.1 | 6.5 |
| 14.5 | 4.2 |
| 14.8 | 4.7 |
| 16.8 | 13.0 |
| 17.7 | 100.0 |
| 18.2 | 19.5 |
| 18.6 | 12.1 |
| 19.5 | 9.9 |
| 20.1 | 43.9 |
| 22.4 | 6.2 |
| 23.5 | 26.5 |
| 24.1 | 32.6 |
| 24.6 | 5.6 |
| 25.7 | 19.0 |
| 26.1 | 3.0 |
| 26.3 | 3.8 |
| 26.7 | 7.2 |
| 27.6 | 4.1 |
| 28.3 | 13.2 |
| 28.7 | 12.9 |
| 29.4 | 8.6 |
| 30.4 | 4.5 |
| 31.7 | 9.3 |

¹ In this and all subsequent tables, the position 2θ is within ± 0.2.

### Compound I-10

After cocrystal polymorph screening experiments described above, obtained solids all showed XRPD patterns. The XRPD of Form A of Compound I-10 is shown in FIG. 25. Table 13 below sets out the X-Ray diffraction peaks observed for Form A of Compound I-10, wherein each value is in degrees 2θ:

TABLE 13
XRPD peak list of L-proline Compound I-10 Form A

| Angle (2-Theta °) ¹ | Intensity (%) |
|---|---|
| 5.1 | 21.8 |
| 6.2 | 64.2 |
| 7.1 | 100.0 |
| 8.9 | 31.1 |
| 10.3 | 7.9 |
| 10.7 | 72.7 |
| 12.3 | 2.5 |
| 14.2 | 18.3 |
| 14.5 | 5.0 |
| 16.4 | 9.0 |
| 18.2 | 44.2 |
| 20.1 | 51.9 |
| 20.5 | 9.3 |
| 21.5 | 32.0 |
| 21.8 | 31.6 |
| 22.0 | 29.2 |
| 22.9 | 18.0 |
| 23.5 | 31.3 |
| 24.5 | 23.9 |
| 25.9 | 11.4 |
| 26.9 | 10.8 |
| 28.5 | 6.4 |
| 31.8 | 5.8 |

¹ In this and all subsequent tables, the position 2θ is within ± 0.2.

### Compound I-11

After cocrystal polymorph screening experiments described above, obtained solids all showed XRPD patterns. The XRPD of Form A of Compound I-11 is shown in FIG. 26. Table 14 below sets out the X-Ray diffraction peaks observed for Form A of Compound I-11, wherein each value is in degrees 2θ:

TABLE 14
XRPD peak list of 4-aminobenzoic acid (Compound I-11) Form A

| Angle (2-Theta °) ¹ | Intensity (%) |
|---|---|
| 4.9 | 68.5 |
| 5.4 | 78.8 |
| 5.7 | 11.1 |
| 7.9 | 27.7 |
| 8.5 | 4.4 |
| 10.8 | 28.8 |
| 11.5 | 9.2 |
| 12.0 | 24.5 |
| 12.7 | 26.1 |
| 13.2 | 35.1 |
| 13.7 | 4.3 |
| 14.5 | 8.2 |
| 14.8 | 19.1 |
| 15.9 | 6.2 |
| 16.4 | 24.0 |
| 17.1 | 22.0 |
| 17.4 | 37.3 |
| 17.6 | 43.7 |
| 18.7 | 32.1 |
| 19.4 | 19.4 |
| 20.1 | 88.7 |
| 20.4 | 37.0 |
| 20.8 | 100.0 |
| 21.5 | 49.8 |
| 21.7 | 49.2 |
| 21.8 | 49.2 |
| 22.2 | 27.0 |
| 23.1 | 63.5 |
| 23.5 | 32.8 |
| 24.0 | 70.8 |
| 24.7 | 69.6 |
| 25.2 | 19.6 |
| 25.4 | 26.6 |
| 25.9 | 34.4 |
| 26.8 | 7.3 |
| 27.1 | 10.8 |
| 27.3 | 4.9 |
| 27.6 | 8.3 |
| 28.0 | 12.3 |
| 28.6 | 23.7 |
| 29.2 | 23.3 |
| 29.9 | 4.5 |
| 30.4 | 12.4 |
| 30.8 | 5.3 |

¹ In this and all subsequent tables, the position 2θ is within ± 0.2.

The XRPD of Form B of Compound I-II is shown in FIG. 27. Table 15 below sets out the X-Ray diffraction peaks observed for Form B of Compound I-11, wherein each value is in degrees 2θ:

TABLE 15
XRPD peak list of 4-aminobenzoic acid (Compound I-11) Form B

| Angle (2-Theta °) ¹ | Intensity (%) |
|---|---|
| 4.9 | 60.6 |
| 5.4 | 3.1 |
| 5.7 | 14.4 |
| 8.5 | 7.5 |
| 10.1 | 2.8 |
| 11.5 | 13.2 |
| 12.7 | 34.1 |
| 13.1 | 31.9 |
| 13.7 | 8.5 |
| 14.5 | 9.3 |
| 14.8 | 11.8 |
| 16.3 | 33.1 |
| 17.0 | 33.7 |
| 17.3 | 13.5 |
| 17.6 | 23.9 |
| 18.6 | 8.3 |
| 19.2 | 20.2 |
| 20.2 | 100.0 |
| 20.8 | 13.6 |
| 21.4 | 7.4 |
| 21.7 | 6.8 |
| 22.1 | 9.4 |
| 22.3 | 7.2 |
| 22.8 | 27.6 |
| 23.2 | 44.1 |
| 23.6 | 27.2 |
| 24.1 | 72.4 |
| 24.7 | 6.8 |
| 25.5 | 23.6 |
| 27.6 | 4.3 |
| 28.0 | 11.7 |
| 28.3 | 6.9 |
| 28.6 | 18.6 |
| 29.3 | 2.0 |

¹ In this and all subsequent tables, the position 2θ is within ± 0.2.

### Example 4—Solubility measurements of cocrystals of (3R,5R)-5-(5-(3-(2,2-difluoroethoxy)-1-methyl-1H-pyrazole-5-carboxamido)-1H-pyrazol-3-yl)tetrahydrofuran-3-yl bicyclo[1.1.1]pentan-1-ylcarbamate

Thermodynamic Solubility—Sufficient sample was suspended in 0.5 ml media for a maximum anticipated concentration of Ca. 10 mg/ml of the free form of the compound. The resulting suspensions were then shaken at 25° C./750 rpm for 24 hours in DI H₂O and at 37° C./750 rpm for 24 hours in simulated fluids. After equilibration, the appearance was noted, and the pH of the saturated solution was measured. Samples were then filtered through a glass ‘C’ fibre filter (Particle retention 1.2 μm), before dilution with buffer as appropriate.

Quantitation was by HPLC with reference to a standard solution of approximately 0.15 mg/ml. Different volumes of the standards and diluted sample solutions were injected. The solubility was calculated using the peak areas determined by integration of the peak found at the same retention time as the principal peak in the standard injection.

The results from the solubility measurements in deionized water (DI water), stimulated fasting state conditions in the gastric fluid (FaSGF), simulated fed state conditions in the small intestines (FeSSIF), and simulated fasting state conditions in the small intestines fluid (FaSSIF) are summarized in Table 16.

TABLE 16
Solubility measurements of cocrystals

| | | | | | Average |
|---|---|---|---|---|---|
| | | | | Solu- | Solu- |
| | | Appear- | Final | bility | bility |
| Form | Media | ance | pH | (mg/ml) | (mg/ml) |
| Com- | DI | Residual solid on | 3.6 | 0.21 | 0.21 |
| pound | Water | top and bottom | 3.6 | 0.21 | |
| I-2 | FaSSGF | | 1.8 | 0.34 | 0.35 |
| Form A | | | 1.7 | 0.35 | |
| | FeSSIF | | 4.6 | 0.92 | 0.89 |
| | | | 4.6 | 0.85 | |
| | FaSSIF | Suspension with | 4.2 | 0.29 | 0.29 |
| | | residual solid | | | |
| Com- | DI | Residual solid on | 3.0 | 0.29 | 0.30 |
| ponud | Water | top and bottom | 3.0 | 0.30 | |
| I-3 | FaSSGF | | 1.8 | 0.38 | 0.39 |
| Form A | | | 1.8 | 0.39 | |
| | FeSSIF | Suspension with | 4.5 | 0.62 | 0.49 |
| | | residual solid | 4.6 | 0.36 | |
| | FaSSIF | | 3.4 | 0.29 | 0.29 |
| Com- | DI | Residual solid on | 2.9 | 0.29 | 0.29 |
| pound | Water | top and bottom | 2.8 | 0.29 | |
| I-4 | FaSSGF | Residual solid | 1.8 | 0.37 | 0.37 |
| Form A | | on top | 1.8 | 0.36 | |
| | FeSSIF | Residual solid on | 4.7 | 0.94 | 0.88 |
| | | top and bottom | 4.7 | 0.82 | |
| | FaSSIF | Suspension with | 3.7 | 0.28 | 0.29 |
| | | residual solid | | | |
| Com- | DI | Residual solid on | 4.3 | 0.28 | 0.28 |
| pound | Water | top and bottom | 4.3 | 0.28 | |
| 1-5 | FaSSGF | | 1.7 | 0.31 | 0.31 |
| Form A | | | 1.7 | 0.31 | |
| | FeSSIF | Suspension with | 4.8 | 0.84 | 0.82 |
| | | residual solid | 4.8 | 0.80 | |
| | FaSSIF | | 4.9 | 0.24 | 0.25 |
| Com- | DI | Residual solid on | 9.1 | 0.33 | 0.33 |
| pound | Water | top and bottom | 8.8 | 0.33 | |
| 1-6 | FaSSGF | Residual solid | 2.6 | 0.32 | 0.32 |
| Form A | | on top | 2.6 | 0.32 | |
| | FeSSIF | Suspension with | 4.9 | 0.66 | 0.70 |
| | | residual solid | 4.9 | 0.73 | |
| | FaSSIF | | 6.4 | 0.31 | 0.31 |

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure

What is claimed:

1. A compound in solid form, wherein the compound is of Formula I-1: 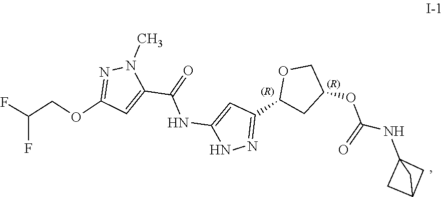
or a solvate thereof.

2. The compound of claim 1, wherein the compound is amorphous.

3. The compound of claim 1, wherein the solid form is crystalline.

4. The compound of claim 3, wherein the solid form is Form A.

5. The compound of claim 3, wherein the solid form is Form B.

6. A compound of Formula (I) 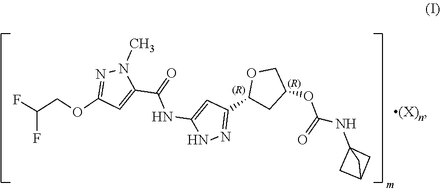
or a solvate thereof,
wherein,
m is 1, 2, 3, 4, 5, 6, 7, 8, or 9,
n is 0, 0.5, 1, 1.5, 2, 2.5, or 3; and
X is succinic acid, urea, nicotinamide, tartaric acid, malic acid, lactic acid, maltol, L-proline, p-toluenesulfonic acid, methane sulfonic acid, benzoic acid, or 4-aminobenzoic acid.

7. The compound of claim 6, wherein the compound is selected from the group consisting of Compound I-2, Compound I-3, Compound I-4, Compound I-5, Compound I-6, Compound I-7, Compound I-8, Compound I-9, Compound I-10, and Compound I-11.

8. The compound of claim 6 or 7, wherein the compound is in solid form.

9. The compound of claim 6 or 7, wherein the compound is crystalline.

10. A pharmaceutical composition comprising a compound of any of claims 1-9 and a pharmaceutically acceptable carrier.

11. A method of inhibiting CDK2 activity in a subject a subject in need thereof, comprising administering a therapeutically effective amount of a compound of any of claims 1-9, or a pharmaceutical composition of claim 10, to the subject.

12. The method of claim 11, further comprising administering a therapeutically effective amount of an antibody, an antibody-drug conjugate, an immunomodulator, or a histone deacetylase inhibitor.

13. The method of claim 11 or 12, wherein the subject is a human.

14. A method of treating a disorder in a subject in need thereof, comprising administering a therapeutically effective amount of a compound of any of claims 1-9, or a pharmaceutical composition of claim 10, to the subject.

15. The method of claim 14, further comprising administration of a therapeutically effective amount of an antibody, an antibody-drug conjugate, an immunomodulator, or a histone deacetylase inhibitor.

16. The method of claim 14 or 15, wherein the disorder is selected from wherein the disorder is ovarian cancer, endometrial cancer, gastric cancer, breast cancer, lung cancer, bladder cancer, cervical cancer, stomach cancer, sarcoma cancer, liver cancer, esophageal cancer, laryngeal cancer, multiple myeloma, colorectal cancer, rectal cancer, skin cancer, or pancreatic cancer.

17. The method of claim 16, wherein the bladder cancer is urothelial carcinoma.

18. The method of claim 16, wherein the liver cancer is hepatocellular carcinoma.

19. The method of claim 16, wherein the lung cancer is lung squamous cell carcinoma or non-small cell lung cancer.

20. The method of claim 16, wherein the laryngeal cancer is laryngeal squamous cell carcinoma.

21. The method of claim 16, wherein the skin cancer is melanoma.

22. A process for preparing the crystalline form of claim 3, comprising: a) preparing a solution of Compound I-1 in a solvent comprising at least one of EtOH, ACN, MEK, EtOAc, IPAc, IPA, THF, MtBE, Toluene, 1,4 dioxane and water, b) heating the solution to completely dissolve the Compound I-1; c) adjusting the temperature so that solid precipitates out of the solution; and d) isolating the crystalline form of Compound I-1.

23. The process of claim 22, wherein the solvent comprises heptane.

24. The process of claim 22, wherein the solvent comprises MEK.

25. The process of claim 22, wherein the solvent comprises IPAc.

26. The process of claim 22, wherein the solvent comprises MEK and heptane.

27. The process of claim 22, wherein the solvent comprises IPAc and heptane.
